SEQUENCE LISTING (38 sequences)

SEQ ID NO: 1 - protein, 360 aa, Homo sapiens
MAGHLASDFAFSPPPGGGGDGPGGPEPGWVDPRTWLSFQGPPGGPGIGPGVGPGSEVWGIPPCPPPYEFCGGMAYCGPQVGVGLVPQGGLETSQPEGEAGVGVESNSDGASPEPCTVTPGAVKLEKEKLEQNPEESQDIKALQKELEQFAKLLKQKRITLGYTQADVGLTLGVLFGKVFSQTTICRFEALQLSFKNMCKLRPLLQKWVEEADNNENLQEICKAETLVQARKRKRTSIENRVRGNLENLFLQCPKPTLQQISHIAQQLGLEKDVVRVWFCNRRQKGKRSSSDYAQREDFEAAGSPFSGGPVSFPLAPGPHFGTPGYGSPHFTALYSSVPFPEGEAFPPVSVTTLGSPMHSN

SEQ ID NO: 2 - protein, 513 aa, Homo sapiens
MRQPPGESDMAVSDALLPSFSTFASGPAGREKTLRQAGAPNNRWREELSHMKRLPPVLPGRPYDLAAATVATDLESGGAGAACGGSNLAPLPRRETEEFNDLLDLDFILSNSLTHPPESVAATVSSSASASSSSSPSSSGPASAPSTCSFTYPIRAGNDPGVAPGGTGGGLLYGRESAPPPTAPFNLADINDVSPSGGFVAELLRPELDPVYIPPQQPQPPGGGLMGKFVLKASLSAPGSEYGSPSVISVSKGSPDGSHPVVVAPYNGGPPRTCPKIKQEAVSSCTHLGAGPPLSNGHRPAAHDFPLGRQLPSRTTPTLGLEEVLSSRDCHPALPLPPGFHPHPGPNYPSFLPDQMQPQVPPLHYQGQSRGFVARAGEPCVCWPHFGTHGMMLTPPSSPLELMPPGSCMPEEPKPKRGRRSWPRKRTATHTCDYAGCGKTYTKSSHLKAHLRTHTGEKPYHCDWDGCGWKFARSDELTRHYRKHTGHRPFQCQKCDRAFSRSDHLALHMKRHF

SEQ ID NO: 3 - protein, 479 aa, Homo sapiens
MRQPPGESDMAVSDALLPSFSTFASGPAGREKTLRQAGAPNNRWREELSHMKRLPPVLPGRPYDLAAATVATDLESGGAGAACGGSNLAPLPRRETEEFNDLLDLDFILSNSLTHPPESVAATVSSSASASSSSSPSSSGPASAPSTCSFTYPIRAGNDPGVAPGGTGGGLLYGRESAPPPTAPFNLADINDVSPSGGFVAELLRPELDPVYIPPQQPQPPGGGLMGKFVLKASLSAPGSEYGSPSVISVSKGSPDGSHPVVVAPYNGGPPRTCPKIKQEAVSSCTHLGAGPPLSNGHRPAAHDFPLGRQLPSRTTPTLGLEEVLSSRDCHPALPLPPGFHPHPGPNYPSFLPDQMQPQVPPLHYQELMPPGSCMPEEPKPKRGRRSWPRKRTATHTCDYAGCGKTYTKSSHLKAHLRTHTGEKPYHCDWDGCGWKFARSDELTRHYRKHTGHRPFQCQKCDRAFSRSDHLALHMKRHF

SEQ ID NO: 4 - protein, 317 aa, Homo sapiens
MYNMMETELKPPGPQQTSGGGGGNSTAAAAGGNQKNSPDRVKRPMNAFMVWSRGQRRKMAQENPKMHNSEISKRLGAEWKLLSETEKRPFIDEAKRLRALHMKEHPDYKYRPRRKTKTLMKKDKYTLPGGLLAPGGNSMASGVGVGAGLGAGVNQRMDSYAHMNGWSNGSYSMMQDQLGYPQHPGLNAHGAAQMQPMHRYDVSALQYNSMTSSQTYMNGSPTYSMSYSQQGTPGMALGSMGSVVKSEASSSPPVVTSSSHSRAPCQAGDLRDMISMYLPGAEVPEPAAPSRLHMSQHYQSGPVPGTAINGTLPLSHM

SEQ ID NO: 5 - protein, 289 aa, Homo sapiens
MSVDPACPQSLPCFEASDCKESSPMPVICGPEENYPSLQMSSAEMPHTETVSPLPSSMDLLIQDSPDSSTSPKGKQPTSAEKSVAKKEDKVPVKKQKTRTVFSSTQLCVLNDRFQRQKYLSLQQMQELSNILNLSYKQVKTWFQNQRMKSKRWQKNNWPKNSNGVTQGCLVNPTGNLPMWSNQTWNNSTWSNQTQNIQSWSNHSWNTQTWCTQSWNNQAWNSPFYNCGEESLQSCMQFQPNSPASDLEAALEAAGEGLNVIQQTTRYFSTPQTMDLFLNYSMNMQPEDV

SEQ ID NO: 6 - protein, 305 aa, Homo sapiens
MSVDPACPQSLPCFEASDCKESSPMPVICGPEENYPSLQMSSAEMPHTETVSPLPSSMDLLIQDSPDSSTSPKGKQPTSAEKSVAKKEDKVPVKKQKTRTVFSSTQLCVLNDRFQRQKYLSLQQMQELSNILNLSYKQVKTWFQNQRMKSKRWQKNNWPKNSNGVTQKASAPTYPSLYSSYHQGCLVNPTGNLPMWSNQTWNNSTWSNQTQNIQSWSNHSWNTQTWCTQSWNNQAWNSPFYNCGEESLQSCMQFQPNSPASDLEAALEAAGEGLNVIQQTTRYFSTPQTMDLFLNYSMNMQPEDV

SEQ ID NO: 7 - protein, 209 aa, Homo sapiens
MGSVSNQQFAGGCAKAAEEAPEEAPEDAARAADEPQLLHGAGICKWFNVRMGFGFLSMTARAGVALDPPVDVFVHQSKLHMEGFRSLKEGEAVEFTFKKSAKGLESIRVTGPGGVFCIGSERRPKGKSMQKRRSKGDRCYNCGGLDHHAKECKLPPQPKKCHFCQSISHMVASCPLKAQQGPSAQGKPTYFREEEEEIHSPTLLPEAQN

SEQ ID NO: 8 - protein, 454 aa, Homo sapiens
MDFFRVVENQQPPATMPLNVSFTNRNYDLDYDSVQPYFYCDEEENFYQQQQQSELQPPAPSEDIWKKFELLPTPPLSPSRRSGLCSPSYVAVTPFSLRGDNDGGGGSFSTADQLEMVTELLGGDMVNQSFICDPDDETFIKNIIIQDCMWSGFSAAAKLVSEKLASYQAARKDSGSPNPARGHSVCSTSSLYLQDLSAAASECIDPSVVFPYPLNDSSSPKSCASQDSSAFSPSSDSLLSSTESSPQGSPEPLVLHEETPPTTSSDSEEEQEDEEEIDVVSVEKRQAPGKRSESGSPSAGGHSKPPHSPLVLKRCHVSTHQHNYAAPPSTRKDYPAAKRVKLDSVRVLRQISNNRKCTSPRSSDTEENVKRRTHNVLERQRRNELKRSFFALRDQIPELENNEKAPKVVILKKATAYILSVQAEEQKLISEEDLLRKRREQLKHKLEQLRNSCA

SEQ ID NO: 9 - protein, 453 aa, Homo sapiens
MDFFRVVENQPPATMPLNVSFTNRNYDLDYDSVQPYFYCDEEENFYQQQQQSELQPPAPSEDIWKKFELLPTPPLSPSRRSGLCSPSYVAVTPFSLRGDNDGGGGSFSTADQLEMVTELLGGDMVNQSFICDPDDETFIKNIIIQDCMWSGFSAAAKLVSEKLASYQAARKDSGSPNPARGHSVCSTSSLYLQDLSAAASECIDPSVVFPYPLNDSSSPKSCASQDSSAFSPSSDSLLSSTESSPQGSPEPLVLHEETPPTTSSDSEEEQEDEEEIDVVSVEKRQAPGKRSESGSPSAGGHSKPPHSPLVLKRCHVSTHQHNYAAPPSTRKDYPAAKRVKLDSVRVLRQISNNRKCTSPRSSDTEENVKRRTHNVLERQRRNELKRSFFALRDQIPELENNEKAPKVVILKKATAYILSVQAEEQKLISEEDLLRKRREQLKHKLEQLRNSCA

SEQ ID NO: 10 - protein, 236 aa, Homo sapiens
MCVCAGCRAAPSRRGAGPLQVAGGWSEGADMDYDSYQHYFYDYDCGEDFYRSTAPSEDIWKKFELVPSPPTSPPWGLGPGAGDPAPGIGPPEPWPGGCTGDEAESRGHSKGWGRNYASIIRRDCMWSGFSARERLERAVSDRLAPGAPRGNPPKASAAPDCTPSLEAGNPAPAAPCPLGEPKTQACSGSESPSDSGKDLPEPSKRGPPHGWPKLCPCLRSGIGSSQALGPSPPLFG

SEQ ID NO: 11 - protein, 364 aa, Homo sapiens
MDYDSYQHYFYDYDCGEDFYRSTAPSEDIWKKFELVPSPPTSPPWGLGPGAGDPAPGIGPPEPWPGGCTGDEAESRGHSKGWGRNYASIIRRDCMWSGFSARERLERAVSDRLAPGAPRGNPPKASAAPDCTPSLEAGNPAPAAPCPLGEPKTQACSGSESPSDSENEEIDVVTVEKRQSLGIRKPVTITVRADPLDPCMKHFHISIHQQQHNYAARFPPESCSQEEASERGPQEEVLERDAAGEKEDEEDEEIVSPPPVESEAAQSCHPKPVSSDTEDVTKRKNHNFLERKRRNDLRSRFLALRDQVPTLASCSKAPKVVILSKALEYLQALVGAEKRMATEKRQLRCRQQQLQKRIAYLTGY

SEQ ID NO: 12 - protein, 394 aa, Homo sapiens
MCVCAGCRAAPSRRGAGPLQVAGGWSEGADMDYDSYQHYFYDYDCGEDFYRSTAPSEDIWKKFELVPSPPTSPPWGLGPGAGDPAPGIGPPEPWPGGCTGDEAESRGHSKGWGRNYASIIRRDCMWSGFSARERLERAVSDRLAPGAPRGNPPKASAAPDCTPSLEAGNPAPAAPCPLGEPKTQACSGSESPSDSENEEIDVVTVEKRQSLGIRKPVTITVRADPLDPCMKHFHISIHQQQHNYAARFPPESCSQEEASERGPQEEVLERDAAGEKEDEEDEEIVSPPPVESEAAQSCHPKPVSSDTEDVTKRKNHNFLERKRRNDLRSRFLALRDQVPTLASCSKAPKVVILSKALEYLQALVGAEKRMATEKRQLRCRQQQLQKRIAYLTGY

SEQ ID NO: 13 - RNA, 320 nt, Homo sapiens
cuaaaccagguggacaggugaaaugucaguauaucucugcuguggauaaaguuauauuuguggaugauuaugcaguaggguguaggaaggaccuuaauggaaucuuguuguuagacacugcucugcaaacuccaguuucaaagcaggaugaugugguucagcuugaauuacccguuacagaggcacagcagcucuuaucagcauguuuagaaaagguagauauuucuaguacagaggguuaugauuuguucaucacacagcucaaagaugguuuaaaaaauacaucucaugagacugcagcaaaccacaaaguugcuaag

SEQ ID NO: 14 - RNA, 251 nt, Homo Sapiens
aaaaauaugcaggaaccaauugcucuucaugagauggacacuagcaaugggguguugcugccuuucuaugacccugacaccagcaucauuuacuuauguggaaagggugacagcaguauucgcuauuuugagaucacggaugaauccccguacguccacuaccucaacacauucagcagcaaggagccucagagagggauggguuacaugcccaagaggggacuugauguuaacaaaugugagauugccag

SEQ ID NO: 15 - RNA, 353 nt, Homo Sapiens
ggaagaggaagaacgucugagaaauaaaauucgagcugaucaugagaaggccuuggaagaagcaaaagaaaaauuaagaaagucaagagaggaaauucgagcagaaauucagacagagaaaaauaagguaguccaagaaaugaagauaaaagagaacaagccacugccaccagucccuauucccaaccuuguaggaauacgugguggagacccagaagauaaugacauaagagagaaaagggaaaaaauuaaagagaugaugaaacaugcuugggauaacuauaggacauauggguggggacauaaugaacucagaccuauugcaaggaaaggacacuccccuaacauauuug

SEQ ID NO: 16 - protein, 351 aa, Vaccinia virus
MTMKMMVHIYFVSLLLLLFHSYAIDIENEITEFFNKMRDTLPAKDSKWLNPACMFGGTMNDIAALGEPFSAKCPPIEDSLLSHRYKDYVVKWERLEKNRRRQVSNKRVKHGDLWIANYTSKFSNRRYLCTVTTKNGDCVQGIVRSHIRKPPSCIPKTYELGTHDKYGIDLYCGILYAKHYNNITWYKDNKEINIDDIKYSQTGKELIIHNPELEDSGRYDCYVHYDDVRIKNDIVVSRCKILTVIPSQDHRFKLILDPKINVTIGEPANITCTAVSTSLLIDDVLIEWENPSGWLIGFDFDVYSVLTSRGGITEATLYFENVTEEYIGNTYKCRGHNYYFEKTLTTTVVLE

SEQ ID NO: 17 - protein, 18 aa, Unknown
  2A peptide
EGRGSLLTCGDVEENPGP

SEQ ID NO: 18 - protein, 19 aa, Unknown
  2A peptide
ATNFSLLKQAGDVEENPGP

SEQ ID NO: 19 - protein, 20 aa, Unknown
  2A peptide
QCTNYALLKLAGDVESNPGP

SEQ ID NO: 20 - protein, 22 aa, Unknown
  2A peptide
VKQTLNFDLLKLAGDVESNPGP

SEQ ID NO: 21 - RNA, 7 nt, Artificial Sequence
  Kozak consensus sequence
rccaugg

SEQ ID NO: 22 - RNA, 6 nt, Artificial Sequence
  Kozak consensus sequence
  misc_feature (2)..(2): n is any ribonucleotide
rnyaug

SEQ ID NO: 23 - RNA, 2039 nt, Artificial Sequence
  precursor RNA encoding nGFP reprogramming factor
gggagacccucgaccgucgauuguccacuggucaacaauagaugacuuacaacuaaucggaaggugcagagacucgacgggagcuacccuaacgucaagacgaggguaaagagagaguccaauucucaaagccaauaggcaguagcgaaagcugcaagagaaugaaaauccguugaccuuaaacggucguguggguucaagucccuccacccccacgccggaaacgcaauagccgaaaaacaaaaaacaaaaaaaacaaaaaaaaaaccaaaaaaacaaaacacauuaaaacagccuguggguugaucccacccacaggcccauugggcgcuagcacucugguaucacgguaccuuugugcgccuguuuuauacccccucccccaacuguaacuuagaaguaacacacaccgaucaacagucagcguggcacaccagccacguuuugaucaagcacuucuguuaccccggacugaguaucaauagacugcucacgcgguugaaggagaaagcguucguuauccggccaacuacuucgaaaaaccuaguaacaccguggaaguugcagaguguuucgcucagcacuaccccaguguagaucaggucgaugagucaccgcauuccccacgggcgaccguggcgguggcugcguuggcggccugcccauggggaaacccaugggacgcucuaauacagacauggugcgaagagucuauugagcuaguugguaguccuccggccccugaaugcggcuaauccuaacugcggagcacacacccucaagccagagggcagugugucguaacgggcaacucugcagcggaaccgacuacuuuggguguccguguuucauuuuauuccuauacuggcugcuuauggugacaauugagagaucguuaccauauagcuauuggauuggccauccggugacuaauagagcuauuauauaucccuuuguuggguuuauaccacuuagcuugaaagagguuaaaacauuacaauucauuguuaaguugaauacagcaaaauggugagcaagggcgaggagcuguucaccgggguggugcccauccuggucgagcuggacggcgacguaaacggccacaaguucagcguguccggcgagggcgagggcgaugccaccuacggcaagcugacccugaaguucaucugcaccaccggcaagcugcccgugcccuggcccacccucgugaccacccugaccuacggcgugcagugcuucagccgcuaccccgaccacaugaagcagcacgacuucuucaaguccgccaugcccgaaggcuacguccaggagcgcaccaucuucuucaaggacgacggcaacuacaagacccgcgccgaggugaaguucgagggcgacacccuggugaaccgcaucgagcugaagggcaucgacuucaaggaggacggcaacauccuggggcacaagcuggaguacaacuacaacagccacaacgucuauaucauggccgacaagcagaagaacggcaucaaggugaacuucaagauccgccacaacaucgaggacggcagcgugcagcucgccgaccacuaccagcagaacacccccaucggcgacggccccgugcugcugcccgacaaccacuaccugagcacccaguccgcccugagcaaagaccccaacgagaagcgcgaucacaugguccugcuggaguucgugaccgccgccgggaucacucucggcauggacgagcuguacaagagaucucgagcugauccaaaaaagaagagaaagguagauccaaaaaagaagagaaagguagauccaaaaaagaagagaaagguauaaaaaaaacaaaaaacaaaacggcuauuaugcguuaccggcgagacgcuacggacuuaaauaauugagccuuaaagaagaaauucuuuaaguggaugcucucaaacucagggaaaccuaaaucuaguuauagacaaggcaauccugagccaagccgaaguaguaauuaguaagaccaguggacaaucgacggauaacagcauaucuaggau

SEQ ID NO: 24 - RNA, 2198 nt, Artificial Sequence
  precursor RNA encoding MyoD reprogramming factor
gggagacccucgaccgucgauuguccacuggucaacaauagaugacuuacaacuaaucggaaggugcagagacucgacgggagcuacccuaacgucaagacgaggguaaagagagaguccaauucucaaagccaauaggcaguagcgaaagcugcaagagaaugaaaauccguugaccuuaaacggucguguggguucaagucccuccacccccacgccggaaacgcaauagccgaaaaacaaaaaacaaaaaaaacaaaaaaaaaaccaaaaaaacaaaacacauuaaaacagccuguggguugaucccacccacaggcccauugggcgcuagcacucugguaucacgguaccuuugugcgccuguuuuauacccccucccccaacuguaacuuagaaguaacacacaccgaucaacagucagcguggcacaccagccacguuuugaucaagcacuucuguuaccccggacugaguaucaauagacugcucacgcgguugaaggagaaagcguucguuauccggccaacuacuucgaaaaaccuaguaacaccguggaaguugcagaguguuucgcucagcacuaccccaguguagaucaggucgaugagucaccgcauuccccacgggcgaccguggcgguggcugcguuggcggccugcccauggggaaacccaugggacgcucuaauacagacauggugcgaagagucuauugagcuaguugguaguccuccggccccugaaugcggcuaauccuaacugcggagcacacacccucaagccagagggcagugugucguaacgggcaacucugcagcggaaccgacuacuuuggguguccguguuucauuuuauuccuauacuggcugcuuauggugacaauugagagaucguuaccauauagcuauuggauuggccauccggugacuaauagagcuauuauauaucccuuuguuggguuuauaccacuuagcuugaaagagguuaaaacauuacaauucauuguuaaguugaauacagcaaaauggagcuacugucgccaccgcuccgcgacguagaccugacggcccccgacggcucucucugcuccuuugccacaacggacgacuucuaugacgacccguguuucgacuccccggaccugcgcuucuucgaagaccuggacccgcgccugaugcacgugggcgcgcuccugaaacccgaagagcacucgcacuuccccgcggcggugcacccggccccgggcgcacgugaggacgagcaugugcgcgcgcccagcgggcaccaccaggcgggccgcugccuacugugggccugcaaggcgugcaagcgcaagaccaccaacgccgaccgccgcaaggccgccaccaugcgcgagcggcgccgccugagcaaaguaaaugaggccuuugagacacucaagcgcugcacgucgagcaauccaaaccagcgguugcccaagguggagauccugcgcaacgccauccgcuauaucgagggccugcaggcucugcugcgcgaccaggacgccgcgcccccuggcgccgcagccgccuucuaugcgccgggcccgcugcccccgggccgcggcggcgagcacuacagcggcgacuccgacgcguccagcccgcgcuccaacugcuccgacggcaugauggacuacagcggccccccgagcggcgcccggcggcggaacugcuacgaaggcgccuacuacaacgaggcgcccagcgaacccaggcccgggaagagugcggcggugucgagccuagacugccuguccagcaucguggagcgcaucuccaccgagagcccugcggcgcccgcccuccugcuggcggacgugccuucugagucgccuccgcgcaggcaagaggcugccgcccccagcgagggagagagcagcggcgaccccacccagucaccggacgccgccccgcagugcccugcgggugcgaaccccaacccgauauaccaggugcucugaaaaaaacaaaaaacaaaacggcuauuaugcguuaccggcgagacgcuacggacuuaaauaauugagccuuaaagaagaaauucuuuaaguggaugcucucaaacucagggaaaccuaaaucuaguuauagacaaggcaauccugagccaagccgaaguaguaauuaguaagaccaguggacaaucgacggauaacagcauaucuaggau

SEQ ID NO: 25 - RNA, 2318 nt, Artificial Sequence
  precursor RNA encoding OCT4 reprogramming factor
gggagacccucgaccgucgauuguccacuggucaacaauagaugacuuacaacuaaucggaaggugcagagacucgacgggagcuacccuaacgucaagacgaggguaaagagagaguccaauucucaaagccaauaggcaguagcgaaagcugcaagagaaugaaaauccguugaccuuaaacggucguguggguucaagucccuccacccccacgccggaaacgcaauagccgaaaaacaaaaaacaaaaaaaacaaaaaaaaaaccaaaaaaacaaaacacauuaaaacagccuguggguugaucccacccacaggcccauugggcgcuagcacucugguaucacgguaccuuugugcgccuguuuuauacccccucccccaacuguaacuuagaaguaacacacaccgaucaacagucagcguggcacaccagccacguuuugaucaagcacuucuguuaccccggacugaguaucaauagacugcucacgcgguugaaggagaaagcguucguuauccggccaacuacuucgaaaaaccuaguaacaccguggaaguugcagaguguuucgcucagcacuaccccaguguagaucaggucgaugagucaccgcauuccccacgggcgaccguggcgguggcugcguuggcggccugcccauggggaaacccaugggacgcucuaauacagacauggugcgaagagucuauugagcuaguugguaguccuccggccccugaaugcggcuaauccuaacugcggagcacacacccucaagccagagggcagugugucguaacgggcaacucugcagcggaaccgacuacuuuggguguccguguuucauuuuauuccuauacuggcugcuuauggugacaauugagagaucguuaccauauagcuauuggauuggccauccggugacuaauagagcuauuauauaucccuuuguuggguuuauaccacuuagcuugaaagagguuaaaacauuacaauucauuguuaaguugaauacagcaaaauggcgggacaccuggcuucggauuucgccuucucgcccccuccaggugguggaggugaugggccaggggggccggagccgggcuggguugauccucggaccuggcuaagcuuccaaggcccuccuggagggccaggaaucgggccggggguugggccaggcucugaggugugggggauucccccaugccccccgccguaugaguucuguggggggauggcguacugugggccccagguuggaguggggcuagugccccaaggcggcuuggagaccucucagccugagggcgaagcaggagucgggguggagagcaacuccgauggggccuccccggagcccugcaccgucaccccuggugccgugaagcuggagaaggagaagcuggagcaaaacccggaggagucccaggacaucaaagcucugcagaaagaacucgagcaauuugccaagcuccugaagcagaagaggaucacccugggauauacacaggccgauguggggcucacccuggggguucuauuugggaagguauucagccaaacgaccaucugccgcuuugaggcucugcagcuuagcuucaagaacauguguaagcugcggcccuugcugcagaaguggguggaggaagcugacaacaaugaaaaucuucaggagauaugcaaagcagaaacccucgugcaggcccgaaagagaaagcgaaccaguaucgagaaccgagugagaggcaaccuggagaauuuguuccugcagugcccgaaacccacacugcagcagaucagccacaucgcccagcagcuugggcucgagaaggaugugguccgagugugguucuguaaccggcgccagaagggcaagcgaucaagcagcgacuaugcacaacgagaggauuuugaggcugcugggucuccuuucucagggggaccaguguccuuuccucuggccccagggccccauuuugguaccccaggcuaugggagcccucacuucacugcacuguacuccucggucccuuucccugagggggaagccuuucccccugucuccgucaccacucugggcucucccaugcauucaaacugaaaaaaacaaaaaacaaaacggcuauuaugcguuaccggcgagacgcuacggacuuaaauaauugagccuuaaagaagaaauucuuuaaguggaugcucucaaacucagggaaaccuaaaucuaguuauagacaaggcaauccugagccaagccgaaguaguaauuaguaagaccaguggacaaucgacggauaacagcauaucuaggau

SEQ ID NO: 26 - RNA, 2189 nt, Artificial Sequence
  precursor RNA encoding SOX2 reprogramming factor
gggagacccucgaccgucgauuguccacuggucaacaauagaugacuuacaacuaaucggaaggugcagagacucgacgggagcuacccuaacgucaagacgaggguaaagagagaguccaauucucaaagccaauaggcaguagcgaaagcugcaagagaaugaaaauccguugaccuuaaacggucguguggguucaagucccuccacccccacgccggaaacgcaauagccgaaaaacaaaaaacaaaaaaaacaaaaaaaaaaccaaaaaaacaaaacacauuaaaacagccuguggguugaucccacccacaggcccauugggcgcuagcacucugguaucacgguaccuuugugcgccuguuuuauacccccucccccaacuguaacuuagaaguaacacacaccgaucaacagucagcguggcacaccagccacguuuugaucaagcacuucuguuaccccggacugaguaucaauagacugcucacgcgguugaaggagaaagcguucguuauccggccaacuacuucgaaaaaccuaguaacaccguggaaguugcagaguguuucgcucagcacuaccccaguguagaucaggucgaugagucaccgcauuccccacgggcgaccguggcgguggcugcguuggcggccugcccauggggaaacccaugggacgcucuaauacagacauggugcgaagagucuauugagcuaguugguaguccuccggccccugaaugcggcuaauccuaacugcggagcacacacccucaagccagagggcagugugucguaacgggcaacucugcagcggaaccgacuacuuuggguguccguguuucauuuuauuccuauacuggcugcuuauggugacaauugagagaucguuaccauauagcuauuggauuggccauccggugacuaauagagcuauuauauaucccuuuguuggguuuauaccacuuagcuugaaagagguuaaaacauuacaauucauuguuaaguugaauacagcaaaauguacaacaugauggagacggagcugaagccgccgggcccgcagcaaacuucggggggcggcggcggcaacuccaccgcggcggcggccggcggcaaccagaaaaacagcccggaccgcgucaagcggcccaugaaugccuucaugguguggucccgcgggcagcggcgcaagauggcccaggagaaccccaagaugcacaacucggagaucagcaagcgccugggcgccgaguggaaacuuuugucggagacggagaagcggccguucaucgacgaggcuaagcggcugcgagcgcugcacaugaaggagcacccggauuauaaauaccggccccggcggaaaaccaagacgcucaugaagaaggauaaguacacgcugcccggcgggcugcuggcccccggcggcaauagcauggcgagcggggucggggugggcgccggccugggcgcgggcgugaaccagcgcauggacaguuacgcgcacaugaacggcuggagcaacggcagcuacagcaugaugcaggaccagcugggcuacccgcagcacccgggccucaaugcgcacggcgcagcgcagaugcagcccaugcaccgcuacgacgugagcgcccugcaguacaacuccaugaccagcucgcagaccuacaugaacggcucgcccaccuacagcauguccuacucgcagcagggcaccccuggcauggcucuuggcuccauggguucgguggucaaguccgaggccagcuccagccccccugugguuaccucuuccucccacuccagggcgcccugccaggccggggaccuccgggacaugaucagcauguaucuccccggcgccgaggugccggaacccgccgcccccagcagacuucacaugucccagcacuaccagagcggcccggugcccggcacggccauuaacggcacacugccccucucacacaugugaaaaaaacaaaaaacaaaacggcuauuaugcguuaccggcgagacgcuacggacuuaaauaauugagccuuaaagaagaaauucuuuaaguggaugcucucaaacucagggaaaccuaaaucuaguuauagacaaggcaauccugagccaagccgaaguaguaauuaguaagaccaguggacaaucgacggauaacagcauaucuaggau

SEQ ID NO: 27 - RNA, 1865 nt, Artificial Sequence
  precursor RNA encoding LIN28 reprogramming factor
gggagacccucgaccgucgauuguccacuggucaacaauagaugacuuacaacuaaucggaaggugcagagacucgacgggagcuacccuaacgucaagacgaggguaaagagagaguccaauucucaaagccaauaggcaguagcgaaagcugcaagagaaugaaaauccguugaccuuaaacggucguguggguucaagucccuccacccccacgccggaaacgcaauagccgaaaaacaaaaaacaaaaaaaacaaaaaaaaaaccaaaaaaacaaaacacauuaaaacagccuguggguugaucccacccacaggcccauugggcgcuagcacucugguaucacgguaccuuugugcgccuguuuuauacccccucccccaacuguaacuuagaaguaacacacaccgaucaacagucagcguggcacaccagccacguuuugaucaagcacuucuguuaccccggacugaguaucaauagacugcucacgcgguugaaggagaaagcguucguuauccggccaacuacuucgaaaaaccuaguaacaccguggaaguugcagaguguuucgcucagcacuaccccaguguagaucaggucgaugagucaccgcauuccccacgggcgaccguggcgguggcugcguuggcggccugcccauggggaaacccaugggacgcucuaauacagacauggugcgaagagucuauugagcuaguugguaguccuccggccccugaaugcggcuaauccuaacugcggagcacacacccucaagccagagggcagugugucguaacgggcaacucugcagcggaaccgacuacuuuggguguccguguuucauuuuauuccuauacuggcugcuuauggugacaauugagagaucguuaccauauagcuauuggauuggccauccggugacuaauagagcuauuauauaucccuuuguuggguuuauaccacuuagcuugaaagagguuaaaacauuacaauucauuguuaaguugaauacagcaaaaugggcuccguguccaaccagcaguuugcagguggcugcgccaaggcggcagaagaggcgcccgaggaggcgccggaggacgcggcccgggcggcggacgagccucagcugcugcacggugcgggcaucuguaagugguucaacgugcgcaugggguucggcuuccuguccaugaccgcccgcgccggggucgcgcucgaccccccaguggaugucuuugugcaccagaguaagcugcacauggaaggguuccggagcuugaaggagggugaggcaguggaguucaccuuuaagaagucagccaagggucuggaauccauccgugucaccggaccugguggaguauucuguauugggagugagaggcggccaaaaggaaagagcaugcagaagcgcagaucaaaaggagacaggugcuacaacuguggaggucuagaucaucaugccaaggaaugcaagcugccaccccagcccaagaagugccacuucugccagagcaucagccauaugguagccucauguccgcugaaggcccagcagggcccuagugcacagggaaagccaaccuacuuucgagaggaagaagaagaaauccacagcccuacccugcucccggaggcacagaauugaaaaaaacaaaaaacaaaacggcuauuaugcguuaccggcgagacgcuacggacuuaaauaauugagccuuaaagaagaaauucuuuaaguggaugcucucaaacucagggaaaccuaaaucuaguuauagacaaggcaauccugagccaagccgaaguaguaauuaguaagaccaguggacaaucgacggauaacagcauaucuaggau

SEQ ID NO: 28 - RNA, 2153 nt, Artificial Sequence
  precursor RNA encoding NANOG reprogramming factor
gggagacccucgaccgucgauuguccacuggucaacaauagaugacuuacaacuaaucggaaggugcagagacucgacgggagcuacccuaacgucaagacgaggguaaagagagaguccaauucucaaagccaauaggcaguagcgaaagcugcaagagaaugaaaauccguugaccuuaaacggucguguggguucaagucccuccacccccacgccggaaacgcaauagccgaaaaacaaaaaacaaaaaaaacaaaaaaaaaaccaaaaaaacaaaacacauuaaaacagccuguggguugaucccacccacaggcccauugggcgcuagcacucugguaucacgguaccuuugugcgccuguuuuauacccccucccccaacuguaacuuagaaguaacacacaccgaucaacagucagcguggcacaccagccacguuuugaucaagcacuucuguuaccccggacugaguaucaauagacugcucacgcgguugaaggagaaagcguucguuauccggccaacuacuucgaaaaaccuaguaacaccguggaaguugcagaguguuucgcucagcacuaccccaguguagaucaggucgaugagucaccgcauuccccacgggcgaccguggcgguggcugcguuggcggccugcccauggggaaacccaugggacgcucuaauacagacauggugcgaagagucuauugagcuaguugguaguccuccggccccugaaugcggcuaauccuaacugcggagcacacacccucaagccagagggcagugugucguaacgggcaacucugcagcggaaccgacuacuuuggguguccguguuucauuuuauuccuauacuggcugcuuauggugacaauugagagaucguuaccauauagcuauuggauuggccauccggugacuaauagagcuauuauauaucccuuuguuggguuuauaccacuuagcuugaaagagguuaaaacauuacaauucauuguuaaguugaauacagcaaaaugaguguggauccagcuuguccccaaagcuugccuugcuuugaagcauccgacuguaaagaaucuucaccuaugccugugauuugugggccugaagaaaacuauccauccuugcaaaugucuucugcugagaugccucacacggagacugucucuccucuuccuuccuccauggaucugcuuauucaggacagcccugauucuuccaccagucccaaaggcaaacaacccacuucugcagagaagagugucgcaaaaaaggaagacaaggucccggucaagaaacagaagaccagaacuguguucucuuccacccagcuguguguacucaaugauagauuucagagacagaaauaccucagccuccagcagaugcaagaacucuccaacauccugaaccucagcuacaaacaggugaagaccugguuccagaaccagagaaugaaaucuaagagguggcagaaaaacaacuggccgaagaauagcaauggugugacgcagaaggccucagcaccuaccuaccccagccuuuacucuuccuaccaccagggaugccuggugaacccgacugggaaccuuccaauguggagcaaccagaccuggaacaauucaaccuggagcaaccagacccagaacauccaguccuggagcaaccacuccuggaacacucagaccuggugcacccaauccuggaacaaucaggccuggaacagucccuucuauaacuguggagaggaaucucugcaguccugcaugcaguuccagccaaauucuccugccagugacuuggaggcugccuuggaagcugcuggggaaggccuuaauguaauacagcagaccacuagguauuuuaguacuccacaaaccauggauuuauuccuaaacuacuccaugaacaugcaaccugaagacgugugaaaaaaacaaaaaacaaaacggcuauuaugcguuaccggcgagacgcuacggacuuaaauaauugagccuuaaagaagaaauucuuuaaguggaugcucucaaacucagggaaaccuaaaucuaguuauagacaaggcaauccugagccaagccgaaguaguaauuaguaagaccaguggacaaucgacggauaacagcauaucuaggau

SEQ ID NO: 29 - RNA, 2675 nt, Artificial Sequence
  precursor RNA encoding KLF4 reprogramming factor
gggagacccucgaccgucgauuguccacuggucaacaauagaugacuuacaacuaaucggaaggugcagagacucgacgggagcuacccuaacgucaagacgaggguaaagagagaguccaauucucaaagccaauaggcaguagcgaaagcugcaagagaaugaaaauccguugaccuuaaacggucguguggguucaagucccuccacccccacgccggaaacgcaauagccgaaaaacaaaaaacaaaaaaaacaaaaaaaaaaccaaaaaaacaaaacacauuaaaacagccuguggguugaucccacccacaggcccauugggcgcuagcacucugguaucacgguaccuuugugcgccuguuuuauacccccucccccaacuguaacuuagaaguaacacacaccgaucaacagucagcguggcacaccagccacguuuugaucaagcacuucuguuaccccggacugaguaucaauagacugcucacgcgguugaaggagaaagcguucguuauccggccaacuacuucgaaaaaccuaguaacaccguggaaguugcagaguguuucgcucagcacuaccccaguguagaucaggucgaugagucaccgcauuccccacgggcgaccguggcgguggcugcguuggcggccugcccauggggaaacccaugggacgcucuaauacagacauggugcgaagagucuauugagcuaguugguaguccuccggccccugaaugcggcuaauccuaacugcggagcacacacccucaagccagagggcagugugucguaacgggcaacucugcagcggaaccgacuacuuuggguguccguguuucauuuuauuccuauacuggcugcuuauggugacaauugagagaucguuaccauauagcuauuggauuggccauccggugacuaauagagcuauuauauaucccuuuguuggguuuauaccacuuagcuugaaagagguuaaaacauuacaauucauuguuaaguugaauacagcaaaaugaggcagccaccuggcgagucugacauggcugucagcgacgcgcugcucccaucuuucuccacguucgcgucuggcccggcgggaagggagaagacacugcgucaagcaggugccccgaauaaccgcuggcgggaggagcucucccacaugaagcgacuucccccagugcuucccggccgccccuaugaccuggcggcggcgaccguggccacagaccuggagagcggcggagccggugcggcuugcggcgguagcaaccuggcgccccuaccucggagagagaccgaggaguucaacgaucuccuggaccuggacuuuauucucuccaauucgcugacccauccuccggagucaguggccgccaccguguccucgucagcgucagccuccucuucgucgucgccgucgagcagcggcccugccagcgcgcccuccaccugcagcuucaccuauccgauccgggccgggaacgacccgggcguggcgccgggcggcacgggcggaggccuccucuauggcagggaguccgcucccccuccgacggcucccuucaaccuggcggacaucaacgacgugagccccucgggcggcuucguggccgagcuccugcggccagaauuggacccgguguacauuccgccgcagcagccgcagccgccagguggcgggcugaugggcaaguucgugcugaaggcgucgcugagcgccccuggcagcgaguacggcagcccgucggucaucagcgucagcaaaggcagcccugacggcagccacccgguggugguggcgcccuacaacggcgggccgccgcgcacgugccccaagaucaagcaggaggcggucucuucgugcacccacuugggcgcuggacccccucucagcaauggccaccggccggcugcacacgacuucccccuggggcggcagcuccccagcaggacuaccccgacccugggucuugaggaagugcugagcagcagggacugucacccugcccugccgcuuccucccggcuuccauccccacccggggcccaauuacccauccuuccugcccgaucagaugcagccgcaagucccgccgcuccauuaccaagagcucaugccacccgguuccugcaugccagaggagcccaagccaaagaggggaagacgaucguggccccggaaaaggaccgccacccacacuugugauuacgcgggcugcggcaaaaccuacacaaagaguucccaucucaaggcacaccugcgaacccacacaggugagaaaccuuaccacugugacugggacggcuguggauggaaauucgcccgcucagaugaacugaccaggcacuaccguaaacacacggggcaccgcccguuccagugccaaaaaugcgaccgagcauuuuccaggucggaccaccucgccuuacacaugaagaggcauuuuuaaaaaaaacaaaaaacaaaacggcuauuaugcguuaccggcgagacgcuacggacuuaaauaauugagccuuaaagaagaaauucuuuaaguggaugcucucaaacucagggaaaccuaaaucuaguuauagacaaggcaauccugagccaagccgaaguaguaauuaguaagaccaguggacaaucgacggauaacagcauaucuaggau

SEQ ID NO: 30 - RNA, 2556 nt, Artificial Sequence
  precursor RNA encoding cMYC reprogramming factor
gggagacccucgaccgucgauuguccacuggucaacaauagaugacuuacaacuaaucggaaggugcagagacucgacgggagcuacccuaacgucaagacgaggguaaagagagaguccaauucucaaagccaauaggcaguagcgaaagcugcaagagaaugaaaauccguugaccuuaaacggucguguggguucaagucccuccacccccacgccggaaacgcaauagccgaaaaacaaaaaacaaaaaaaacaaaaaaaaaaccaaaaaaacaaaacacauuaaaacagccuguggguugaucccacccacaggcccauugggcgcuagcacucugguaucacgguaccuuugugcgccuguuuuauacccccucccccaacuguaacuuagaaguaacacacaccgaucaacagucagcguggcacaccagccacguuuugaucaagcacuucuguuaccccggacugaguaucaauagacugcucacgcgguugaaggagaaagcguucguuauccggccaacuacuucgaaaaaccuaguaacaccguggaaguugcagaguguuucgcucagcacuaccccaguguagaucaggucgaugagucaccgcauuccccacgggcgaccguggcgguggcugcguuggcggccugcccauggggaaacccaugggacgcucuaauacagacauggugcgaagagucuauugagcuaguugguaguccuccggccccugaaugcggcuaauccuaacugcggagcacacacccucaagccagagggcagugugucguaacgggcaacucugcagcggaaccgacuacuuuggguguccguguuucauuuuauuccuauacuggcugcuuauggugacaauugagagaucguuaccauauagcuauuggauuggccauccggugacuaauagagcuauuauauaucccuuuguuggguuuauaccacuuagcuugaaagagguuaaaacauuacaauucauuguuaaguugaauacagcaaaaugccccucaacguuagcuucaccaacaggaacuaugaccucgacuacgacucggugcagccguauuucuacugcgacgaggaggagaacuucuaccagcagcagcagcagagcgagcugcagcccccggcgcccagcgaggauaucuggaagaaauucgagcugcugcccaccccgccccuguccccuagccgccgcuccgggcucugcucgcccuccuacguugcggucacacccuucucccuucggggagacaacgacggcgguggcgggagcuucuccacggccgaccagcuggagauggugaccgagcugcugggaggagacauggugaaccagaguuucaucugcgacccggacgacgagaccuucaucaaaaacaucaucauccaggacuguauguggagcggcuucucggccgccgccaagcucgucucagagaagcuggccuccuaccaggcugcgcgcaaagacagcggcagcccgaaccccgcccgcggccacagcgucugcuccaccuccagcuuguaccugcaggaucugagcgccgccgccucagagugcaucgaccccucgguggucuuccccuacccucucaacgacagcagcucgcccaaguccugcgccucgcaagacuccagcgccuucucuccguccucggauucucugcucuccucgacggaguccuccccgcagggcagccccgagccccuggugcuccaugaggagacaccgcccaccaccagcagcgacucugaggaggaacaagaagaugaggaagaaaucgauguuguuucuguggaaaagaggcaggcuccuggcaaaaggucagagucuggaucaccuucugcuggaggccacagcaaaccuccucacagcccacugguccucaagaggugccacgucuccacacaucagcacaacuacgcagcgccucccuccacucggaaggacuauccugcugccaagagggucaaguuggacagugucagaguccugagacagaucagcaacaaccgaaaaugcaccagccccagguccucggacaccgaggagaaugucaagaggcgaacacacaacgucuuggagcgccagaggaggaacgagcuaaaacggagcuuuuuugcccugcgugaccagaucccggaguuggaaaacaaugaaaaggcccccaagguaguuauccuuaaaaaagccacagcauacauccuguccguccaagcagaggagcaaaagcucauuucugaagaggacuuguugcggaaacgacgagaacaguugaaacacaaacuugaacagcuacggaacucuugugcguaaaaaaaacaaaaaacaaaacggcuauuaugcguuaccggcgagacgcuacggacuuaaauaauugagccuuaaagaagaaauucuuuaaguggaugcucucaaacucagggaaaccuaaaucuaguuauagacaaggcaauccugagccaagccgaaguaguaauuaguaagaccaguggacaaucgacggauaacagcauaucuagguuu

SEQ ID NO: 31 - RNA, 1721 nt, Artificial Sequence
  circularized RNA encoding nGFP reprogramming factor
aaaauccguugaccuuaaacggucguguggguucaagucccuccacccccacgccggaaacgcaauagccgaaaaacaaaaaacaaaaaaaacaaaaaaaaaaccaaaaaaacaaaacacauuaaaacagccuguggguugaucccacccacaggcccauugggcgcuagcacucugguaucacgguaccuuugugcgccuguuuuauacccccucccccaacuguaacuuagaaguaacacacaccgaucaacagucagcguggcacaccagccacguuuugaucaagcacuucuguuaccccggacugaguaucaauagacugcucacgcgguugaaggagaaagcguucguuauccggccaacuacuucgaaaaaccuaguaacaccguggaaguugcagaguguuucgcucagcacuaccccaguguagaucaggucgaugagucaccgcauuccccacgggcgaccguggcgguggcugcguuggcggccugcccauggggaaacccaugggacgcucuaauacagacauggugcgaagagucuauugagcuaguugguaguccuccggccccugaaugcggcuaauccuaacugcggagcacacacccucaagccagagggcagugugucguaacgggcaacucugcagcggaaccgacuacuuuggguguccguguuucauuuuauuccuauacuggcugcuuauggugacaauugagagaucguuaccauauagcuauuggauuggccauccggugacuaauagagcuauuauauaucccuuuguuggguuuauaccacuuagcuugaaagagguuaaaacauuacaauucauuguuaaguugaauacagcaaaauggugagcaagggcgaggagcuguucaccgggguggugcccauccuggucgagcuggacggcgacguaaacggccacaaguucagcguguccggcgagggcgagggcgaugccaccuacggcaagcugacccugaaguucaucugcaccaccggcaagcugcccgugcccuggcccacccucgugaccacccugaccuacggcgugcagugcuucagccgcuaccccgaccacaugaagcagcacgacuucuucaaguccgccaugcccgaaggcuacguccaggagcgcaccaucuucuucaaggacgacggcaacuacaagacccgcgccgaggugaaguucgagggcgacacccuggugaaccgcaucgagcugaagggcaucgacuucaaggaggacggcaacauccuggggcacaagcuggaguacaacuacaacagccacaacgucuauaucauggccgacaagcagaagaacggcaucaaggugaacuucaagauccgccacaacaucgaggacggcagcgugcagcucgccgaccacuaccagcagaacacccccaucggcgacggccccgugcugcugcccgacaaccacuaccugagcacccaguccgcccugagcaaagaccccaacgagaagcgcgaucacaugguccugcuggaguucgugaccgccgccgggaucacucucggcauggacgagcuguacaagagaucucgagcugauccaaaaaagaagagaaagguagauccaaaaaagaagagaaagguagauccaaaaaagaagagaaagguauaaaaaaaacaaaaaacaaaacggcuauuaugcguuaccggcgagacgcuacggacuu

SEQ ID NO: 32 - RNA, 1880 nt, Artificial Sequence
  circularized RNA encoding MYOD reprogramming factor
aaaauccguugaccuuaaacggucguguggguucaagucccuccacccccacgccggaaacgcaauagccgaaaaacaaaaaacaaaaaaaacaaaaaaaaaaccaaaaaaacaaaacacauuaaaacagccuguggguugaucccacccacaggcccauugggcgcuagcacucugguaucacgguaccuuugugcgccuguuuuauacccccucccccaacuguaacuuagaaguaacacacaccgaucaacagucagcguggcacaccagccacguuuugaucaagcacuucuguuaccccggacugaguaucaauagacugcucacgcgguugaaggagaaagcguucguuauccggccaacuacuucgaaaaaccuaguaacaccguggaaguugcagaguguuucgcucagcacuaccccaguguagaucaggucgaugagucaccgcauuccccacgggcgaccguggcgguggcugcguuggcggccugcccauggggaaacccaugggacgcucuaauacagacauggugcgaagagucuauugagcuaguugguaguccuccggccccugaaugcggcuaauccuaacugcggagcacacacccucaagccagagggcagugugucguaacgggcaacucugcagcggaaccgacuacuuuggguguccguguuucauuuuauuccuauacuggcugcuuauggugacaauugagagaucguuaccauauagcuauuggauuggccauccggugacuaauagagcuauuauauaucccuuuguuggguuuauaccacuuagcuugaaagagguuaaaacauuacaauucauuguuaaguugaauacagcaaaauggagcuacugucgccaccgcuccgcgacguagaccugacggcccccgacggcucucucugcuccuuugccacaacggacgacuucuaugacgacccguguuucgacuccccggaccugcgcuucuucgaagaccuggacccgcgccugaugcacgugggcgcgcuccugaaacccgaagagcacucgcacuuccccgcggcggugcacccggccccgggcgcacgugaggacgagcaugugcgcgcgcccagcgggcaccaccaggcgggccgcugccuacugugggccugcaaggcgugcaagcgcaagaccaccaacgccgaccgccgcaaggccgccaccaugcgcgagcggcgccgccugagcaaaguaaaugaggccuuugagacacucaagcgcugcacgucgagcaauccaaaccagcgguugcccaagguggagauccugcgcaacgccauccgcuauaucgagggccugcaggcucugcugcgcgaccaggacgccgcgcccccuggcgccgcagccgccuucuaugcgccgggcccgcugcccccgggccgcggcggcgagcacuacagcggcgacuccgacgcguccagcccgcgcuccaacugcuccgacggcaugauggacuacagcggccccccgagcggcgcccggcggcggaacugcuacgaaggcgccuacuacaacgaggcgcccagcgaacccaggcccgggaagagugcggcggugucgagccuagacugccuguccagcaucguggagcgcaucuccaccgagagcccugcggcgcccgcccuccugcuggcggacgugccuucugagucgccuccgcgcaggcaagaggcugccgcccccagcgagggagagagcagcggcgaccccacccagucaccggacgccgccccgcagugcccugcgggugcgaaccccaacccgauauaccaggugcucugaaaaaaacaaaaaacaaaacggcuauuaugcguuaccggcgagacgcuacggacuu

SEQ ID NO: 33 - RNA, 2000 nt, Artificial Sequence
  circularized RNA encoding OCT4 reprogramming factor
aaaauccguugaccuuaaacggucguguggguucaagucccuccacccccacgccggaaacgcaauagccgaaaaacaaaaaacaaaaaaaacaaaaaaaaaaccaaaaaaacaaaacacauuaaaacagccuguggguugaucccacccacaggcccauugggcgcuagcacucugguaucacgguaccuuugugcgccuguuuuauacccccucccccaacuguaacuuagaaguaacacacaccgaucaacagucagcguggcacaccagccacguuuugaucaagcacuucuguuaccccggacugaguaucaauagacugcucacgcgguugaaggagaaagcguucguuauccggccaacuacuucgaaaaaccuaguaacaccguggaaguugcagaguguuucgcucagcacuaccccaguguagaucaggucgaugagucaccgcauuccccacgggcgaccguggcgguggcugcguuggcggccugcccauggggaaacccaugggacgcucuaauacagacauggugcgaagagucuauugagcuaguugguaguccuccggccccugaaugcggcuaauccuaacugcggagcacacacccucaagccagagggcagugugucguaacgggcaacucugcagcggaaccgacuacuuuggguguccguguuucauuuuauuccuauacuggcugcuuauggugacaauugagagaucguuaccauauagcuauuggauuggccauccggugacuaauagagcuauuauauaucccuuuguuggguuuauaccacuuagcuugaaagagguuaaaacauuacaauucauuguuaaguugaauacagcaaaauggcgggacaccuggcuucggauuucgccuucucgcccccuccaggugguggaggugaugggccaggggggccggagccgggcuggguugauccucggaccuggcuaagcuuccaaggcccuccuggagggccaggaaucgggccggggguugggccaggcucugaggugugggggauucccccaugccccccgccguaugaguucuguggggggauggcguacugugggccccagguuggaguggggcuagugccccaaggcggcuuggagaccucucagccugagggcgaagcaggagucgggguggagagcaacuccgauggggccuccccggagcccugcaccgucaccccuggugccgugaagcuggagaaggagaagcuggagcaaaacccggaggagucccaggacaucaaagcucugcagaaagaacucgagcaauuugccaagcuccugaagcagaagaggaucacccugggauauacacaggccgauguggggcucacccuggggguucuauuugggaagguauucagccaaacgaccaucugccgcuuugaggcucugcagcuuagcuucaagaacauguguaagcugcggcccuugcugcagaaguggguggaggaagcugacaacaaugaaaaucuucaggagauaugcaaagcagaaacccucgugcaggcccgaaagagaaagcgaaccaguaucgagaaccgagugagaggcaaccuggagaauuuguuccugcagugcccgaaacccacacugcagcagaucagccacaucgcccagcagcuugggcucgagaaggaugugguccgagugugguucuguaaccggcgccagaagggcaagcgaucaagcagcgacuaugcacaacgagaggauuuugaggcugcugggucuccuuucucagggggaccaguguccuuuccucuggccccagggccccauuuugguaccccaggcuaugggagcccucacuucacugcacuguacuccucggucccuuucccugagggggaagccuuucccccugucuccgucaccacucugggcucucccaugcauucaaacugaaaaaaacaaaaaacaaaacggcuauuaugcguuaccggcgagacgcuacggacuu

SEQ ID NO: 34 - RNA, 1871 nt, Artificial Sequence
  circularized RNA encoding SOX2 reprogramming factor
aaaauccguugaccuuaaacggucguguggguucaagucccuccacccccacgccggaaacgcaauagccgaaaaacaaaaaacaaaaaaaacaaaaaaaaaaccaaaaaaacaaaacacauuaaaacagccuguggguugaucccacccacaggcccauugggcgcuagcacucugguaucacgguaccuuugugcgccuguuuuauacccccucccccaacuguaacuuagaaguaacacacaccgaucaacagucagcguggcacaccagccacguuuugaucaagcacuucuguuaccccggacugaguaucaauagacugcucacgcgguugaaggagaaagcguucguuauccggccaacuacuucgaaaaaccuaguaacaccguggaaguugcagaguguuucgcucagcacuaccccaguguagaucaggucgaugagucaccgcauuccccacgggcgaccguggcgguggcugcguuggcggccugcccauggggaaacccaugggacgcucuaauacagacauggugcgaagagucuauugagcuaguugguaguccuccggccccugaaugcggcuaauccuaacugcggagcacacacccucaagccagagggcagugugucguaacgggcaacucugcagcggaaccgacuacuuuggguguccguguuucauuuuauuccuauacuggcugcuuauggugacaauugagagaucguuaccauauagcuauuggauuggccauccggugacuaauagagcuauuauauaucccuuuguuggguuuauaccacuuagcuugaaagagguuaaaacauuacaauucauuguuaaguugaauacagcaaaauguacaacaugauggagacggagcugaagccgccgggcccgcagcaaacuucggggggcggcggcggcaacuccaccgcggcggcggccggcggcaaccagaaaaacagcccggaccgcgucaagcggcccaugaaugccuucaugguguggucccgcgggcagcggcgcaagauggcccaggagaaccccaagaugcacaacucggagaucagcaagcgccugggcgccgaguggaaacuuuugucggagacggagaagcggccguucaucgacgaggcuaagcggcugcgagcgcugcacaugaaggagcacccggauuauaaauaccggccccggcggaaaaccaagacgcucaugaagaaggauaaguacacgcugcccggcgggcugcuggcccccggcggcaauagcauggcgagcggggucggggugggcgccggccugggcgcgggcgugaaccagcgcauggacaguuacgcgcacaugaacggcuggagcaacggcagcuacagcaugaugcaggaccagcugggcuacccgcagcacccgggccucaaugcgcacggcgcagcgcagaugcagcccaugcaccgcuacgacgugagcgcccugcaguacaacuccaugaccagcucgcagaccuacaugaacggcucgcccaccuacagcauguccuacucgcagcagggcaccccuggcauggcucuuggcuccauggguucgguggucaaguccgaggccagcuccagccccccugugguuaccucuuccucccacuccagggcgcccugccaggccggggaccuccgggacaugaucagcauguaucuccccggcgccgaggugccggaacccgccgcccccagcagacuucacaugucccagcacuaccagagcggcccggugcccggcacggccauuaacggcacacugccccucucacacaugugaaaaaaacaaaaaacaaaacggcuauuaugcguuaccggcgagacgcuacggacuu

SEQ ID NO: 35 - RNA, 1547 nt, Artificial Sequence
  circularized RNA encoding LIN28 reprogramming factor
aaaauccguugaccuuaaacggucguguggguucaagucccuccacccccacgccggaaacgcaauagccgaaaaacaaaaaacaaaaaaaacaaaaaaaaaaccaaaaaaacaaaacacauuaaaacagccuguggguugaucccacccacaggcccauugggcgcuagcacucugguaucacgguaccuuugugcgccuguuuuauacccccucccccaacuguaacuuagaaguaacacacaccgaucaacagucagcguggcacaccagccacguuuugaucaagcacuucuguuaccccggacugaguaucaauagacugcucacgcgguugaaggagaaagcguucguuauccggccaacuacuucgaaaaaccuaguaacaccguggaaguugcagaguguuucgcucagcacuaccccaguguagaucaggucgaugagucaccgcauuccccacgggcgaccguggcgguggcugcguuggcggccugcccauggggaaacccaugggacgcucuaauacagacauggugcgaagagucuauugagcuaguugguaguccuccggccccugaaugcggcuaauccuaacugcggagcacacacccucaagccagagggcagugugucguaacgggcaacucugcagcggaaccgacuacuuuggguguccguguuucauuuuauuccuauacuggcugcuuauggugacaauugagagaucguuaccauauagcuauuggauuggccauccggugacuaauagagcuauuauauaucccuuuguuggguuuauaccacuuagcuugaaagagguuaaaacauuacaauucauuguuaaguugaauacagcaaaaugggcuccguguccaaccagcaguuugcagguggcugcgccaaggcggcagaagaggcgcccgaggaggcgccggaggacgcggcccgggcggcggacgagccucagcugcugcacggugcgggcaucuguaagugguucaacgugcgcaugggguucggcuuccuguccaugaccgcccgcgccggggucgcgcucgaccccccaguggaugucuuugugcaccagaguaagcugcacauggaaggguuccggagcuugaaggagggugaggcaguggaguucaccuuuaagaagucagccaagggucuggaauccauccgugucaccggaccugguggaguauucuguauugggagugagaggcggccaaaaggaaagagcaugcagaagcgcagaucaaaaggagacaggugcuacaacuguggaggucuagaucaucaugccaaggaaugcaagcugccaccccagcccaagaagugccacuucugccagagcaucagccauaugguagccucauguccgcugaaggcccagcagggcccuagugcacagggaaagccaaccuacuuucgagaggaagaagaagaaauccacagcccuacccugcucccggaggcacagaauugaaaaaaacaaaaaacaaaacggcuauuaugcguuaccggcgagacgcuacggacuu

SEQ ID NO: 36 - RNA, 1835 nt, Artificial Sequence
  circularized RNA encoding NANOG reprogramming factor
aaaauccguugaccuuaaacggucguguggguucaagucccuccacccccacgccggaaacgcaauagccgaaaaacaaaaaacaaaaaaaacaaaaaaaaaaccaaaaaaacaaaacacauuaaaacagccuguggguugaucccacccacaggcccauugggcgcuagcacucugguaucacgguaccuuugugcgccuguuuuauacccccucccccaacuguaacuuagaaguaacacacaccgaucaacagucagcguggcacaccagccacguuuugaucaagcacuucuguuaccccggacugaguaucaauagacugcucacgcgguugaaggagaaagcguucguuauccggccaacuacuucgaaaaaccuaguaacaccguggaaguugcagaguguuucgcucagcacuaccccaguguagaucaggucgaugagucaccgcauuccccacgggcgaccguggcgguggcugcguuggcggccugcccauggggaaacccaugggacgcucuaauacagacauggugcgaagagucuauugagcuaguugguaguccuccggccccugaaugcggcuaauccuaacugcggagcacacacccucaagccagagggcagugugucguaacgggcaacucugcagcggaaccgacuacuuuggguguccguguuucauuuuauuccuauacuggcugcuuauggugacaauugagagaucguuaccauauagcuauuggauuggccauccggugacuaauagagcuauuauauaucccuuuguuggguuuauaccacuuagcuugaaagagguuaaaacauuacaauucauuguuaaguugaauacagcaaaaugaguguggauccagcuuguccccaaagcuugccuugcuuugaagcauccgacuguaaagaaucuucaccuaugccugugauuugugggccugaagaaaacuauccauccuugcaaaugucuucugcugagaugccucacacggagacugucucuccucuuccuuccuccauggaucugcuuauucaggacagcccugauucuuccaccagucccaaaggcaaacaacccacuucugcagagaagagugucgcaaaaaaggaagacaaggucccggucaagaaacagaagaccagaacuguguucucuuccacccagcuguguguacucaaugauagauuucagagacagaaauaccucagccuccagcagaugcaagaacucuccaacauccugaaccucagcuacaaacaggugaagaccugguuccagaaccagagaaugaaaucuaagagguggcagaaaaacaacuggccgaagaauagcaauggugugacgcagaaggccucagcaccuaccuaccccagccuuuacucuuccuaccaccagggaugccuggugaacccgacugggaaccuuccaauguggagcaaccagaccuggaacaauucaaccuggagcaaccagacccagaacauccaguccuggagcaaccacuccuggaacacucagaccuggugcacccaauccuggaacaaucaggccuggaacagucccuucuauaacuguggagaggaaucucugcaguccugcaugcaguuccagccaaauucuccugccagugacuuggaggcugccuuggaagcugcuggggaaggccuuaauguaauacagcagaccacuagguauuuuaguacuccacaaaccauggauuuauuccuaaacuacuccaugaacaugcaaccugaagacgugugaaaaaaacaaaaaacaaaacggcuauuaugcguuaccggcgagacgcuacggacuu

SEQ ID NO: 37 - RNA, 2357 nt, Artificial Sequence
  circularized RNA encoding KLF4 reprogramming factor
aaaauccguugaccuuaaacggucguguggguucaagucccuccacccccacgccggaaacgcaauagccgaaaaacaaaaaacaaaaaaaacaaaaaaaaaaccaaaaaaacaaaacacauuaaaacagccuguggguugaucccacccacaggcccauugggcgcuagcacucugguaucacgguaccuuugugcgccuguuuuauacccccucccccaacuguaacuuagaaguaacacacaccgaucaacagucagcguggcacaccagccacguuuugaucaagcacuucuguuaccccggacugaguaucaauagacugcucacgcgguugaaggagaaagcguucguuauccggccaacuacuucgaaaaaccuaguaacaccguggaaguugcagaguguuucgcucagcacuaccccaguguagaucaggucgaugagucaccgcauuccccacgggcgaccguggcgguggcugcguuggcggccugcccauggggaaacccaugggacgcucuaauacagacauggugcgaagagucuauugagcuaguugguaguccuccggccccugaaugcggcuaauccuaacugcggagcacacacccucaagccagagggcagugugucguaacgggcaacucugcagcggaaccgacuacuuuggguguccguguuucauuuuauuccuauacuggcugcuuauggugacaauugagagaucguuaccauauagcuauuggauuggccauccggugacuaauagagcuauuauauaucccuuuguuggguuuauaccacuuagcuugaaagagguuaaaacauuacaauucauuguuaaguugaauacagcaaaaugaggcagccaccuggcgagucugacauggcugucagcgacgcgcugcucccaucuuucuccacguucgcgucuggcccggcgggaagggagaagacacugcgucaagcaggugccccgaauaaccgcuggcgggaggagcucucccacaugaagcgacuucccccagugcuucccggccgccccuaugaccuggcggcggcgaccguggccacagaccuggagagcggcggagccggugcggcuugcggcgguagcaaccuggcgccccuaccucggagagagaccgaggaguucaacgaucuccuggaccuggacuuuauucucuccaauucgcugacccauccuccggagucaguggccgccaccguguccucgucagcgucagccuccucuucgucgucgccgucgagcagcggcccugccagcgcgcccuccaccugcagcuucaccuauccgauccgggccgggaacgacccgggcguggcgccgggcggcacgggcggaggccuccucuauggcagggaguccgcucccccuccgacggcucccuucaaccuggcggacaucaacgacgugagccccucgggcggcuucguggccgagcuccugcggccagaauuggacccgguguacauuccgccgcagcagccgcagccgccagguggcgggcugaugggcaaguucgugcugaaggcgucgcugagcgccccuggcagcgaguacggcagcccgucggucaucagcgucagcaaaggcagcccugacggcagccacccgguggugguggcgcccuacaacggcgggccgccgcgcacgugccccaagaucaagcaggaggcggucucuucgugcacccacuugggcgcuggacccccucucagcaauggccaccggccggcugcacacgacuucccccuggggcggcagcuccccagcaggacuaccccgacccugggucuugaggaagugcugagcagcagggacugucacccugcccugccgcuuccucccggcuuccauccccacccggggcccaauuacccauccuuccugcccgaucagaugcagccgcaagucccgccgcuccauuaccaagagcucaugccacccgguuccugcaugccagaggagcccaagccaaagaggggaagacgaucguggccccggaaaaggaccgccacccacacuugugauuacgcgggcugcggcaaaaccuacacaaagaguucccaucucaaggcacaccugcgaacccacacaggugagaaaccuuaccacugugacugggacggcuguggauggaaauucgcccgcucagaugaacugaccaggcacuaccguaaacacacggggcaccgcccguuccagugccaaaaaugcgaccgagcauuuuccaggucggaccaccucgccuuacacaugaagaggcauuuuuaaaaaaaacaaaaaacaaaacggcuauuaugcguuaccggcgagacgcuacggacuu

SEQ ID NO: 38 - RNA, 2237 nt, Artificial Sequence
  circularized RNA encoding cMYC reprogramming factor
aaaauccguugaccuuaaacggucguguggguucaagucccuccacccccacgccggaaacgcaauagccgaaaaacaaaaaacaaaaaaaacaaaaaaaaaaccaaaaaaacaaaacacauuaaaacagccuguggguugaucccacccacaggcccauugggcgcuagcacucugguaucacgguaccuuugugcgccuguuuuauacccccucccccaacuguaacuuagaaguaacacacaccgaucaacagucagcguggcacaccagccacguuuugaucaagcacuucuguuaccccggacugaguaucaauagacugcucacgcgguugaaggagaaagcguucguuauccggccaacuacuucgaaaaaccuaguaacaccguggaaguugcagaguguuucgcucagcacuaccccaguguagaucaggucgaugagucaccgcauuccccacgggcgaccguggcgguggcugcguuggcggccugcccauggggaaacccaugggacgcucuaauacagacauggugcgaagagucuauugagcuaguugguaguccuccggccccugaaugcggcuaauccuaacugcggagcacacacccucaagccagagggcagugugucguaacgggcaacucugcagcggaaccgacuacuuuggguguccguguuucauuuuauuccuauacuggcugcuuauggugacaauugagagaucguuaccauauagcuauuggauuggccauccggugacuaauagagcuauuauauaucccuuuguuggguuuauaccacuuagcuugaaagagguuaaaacauuacaauucauuguuaaguugaauacagcaaaaugccccucaacguuagcuucaccaacaggaacuaugaccucgacuacgacucggugcagccguauuucuacugcgacgaggaggagaacuucuaccagcagcagcagcagagcgagcugcagcccccggcgcccagcgaggauaucuggaagaaauucgagcugcugcccaccccgccccuguccccuagccgccgcuccgggcucugcucgcccuccuacguugcggucacacccuucucccuucggggagacaacgacggcgguggcgggagcuucuccacggccgaccagcuggagauggugaccgagcugcugggaggagacauggugaaccagaguuucaucugcgacccggacgacgagaccuucaucaaaaacaucaucauccaggacuguauguggagcggcuucucggccgccgccaagcucgucucagagaagcuggccuccuaccaggcugcgcgcaaagacagcggcagcccgaaccccgcccgcggccacagcgucugcuccaccuccagcuuguaccugcaggaucugagcgccgccgccucagagugcaucgaccccucgguggucuuccccuacccucucaacgacagcagcucgcccaaguccugcgccucgcaagacuccagcgccuucucuccguccucggauucucugcucuccucgacggaguccuccccgcagggcagccccgagccccuggugcuccaugaggagacaccgcccaccaccagcagcgacucugaggaggaacaagaagaugaggaagaaaucgauguuguuucuguggaaaagaggcaggcuccuggcaaaaggucagagucuggaucaccuucugcuggaggccacagcaaaccuccucacagcccacugguccucaagaggugccacgucuccacacaucagcacaacuacgcagcgccucccuccacucggaaggacuauccugcugccaagagggucaaguuggacagugucagaguccugagacagaucagcaacaaccgaaaaugcaccagccccagguccucggacaccgaggagaaugucaagaggcgaacacacaacgucuuggagcgccagaggaggaacgagcuaaaacggagcuuuuuugcccugcgugaccagaucccggaguuggaaaacaaugaaaaggcccccaagguaguuauccuuaaaaaagccacagcauacauccuguccguccaagcagaggagcaaaagcucauuucugaagaggacuuguugcggaaacgacgagaacaguugaaacacaaacuugaacagcuacggaacucuugugcguaaaaaaaacaaaaaacaaaacggcuauuaugcguuaccggcgagacgcuacggacuu

(19) United States
(12) Patent Application Publication
(10) Pub. No.: US 2023/0332182 A1
(43) Pub. Date: Oct. 19, 2023

(54) COMPOSITIONS AND METHODS FOR CELLULAR REPROGRAMMING USING CIRCULAR RNA

(71) Applicant: ElevateBio Technologies, Inc., Cambridge, MA (US)

(72) Inventors: Santosh NARAYAN, Cambridge, MA (US); Austin THIEL, Southborough, MA (US); Melissa CARPENTER, Black Diamond, WA (US); Mitchell Howard FINER, Cambridge, MA (US); Miranda YANG, Cambridge, MA (US); Cherylene PLEWA, Cambridge, MA (US)

(21) Appl. No.: 18/004,151

(22) PCT Filed: Jul. 1, 2021
(86) PCT No.: PCT/US2021/040094

§ 371 Date: Jan. 3, 2023

Related U.S. Application Data
(60) Provisional application No. 63/046,976, filed on Jul. 1, 2020.

(51) Int. Cl. C12N 15/88; C12N 5/074; C12N 5/071; C12N 15/85

(52) U.S. Cl. CPC C12N 15/88; C12N 5/0696; C12N 5/0625; C12N 15/85; C12N 2506/13; C12N 2800/107

(57) ABSTRACT

Provided herein are recombinant circular RNAs comprising at least one protein-coding nucleic acid sequence, wherein the protein-coding nucleic acid sequence encodes a reprogramming factor (e.g., a transcription factor), wherein the reprogramming factor is Oct3/4, Klf4, Sox2, Nanog, Lin28, c-Myc, or L-Myc, or a fragment or variant thereof. Also provided herein are methods of producing induced pluripotent stem cells (iPSC), the method comprising contacting a somatic cell with at least one of the recombinant circular RNAs described herein and maintaining the cell under conditions under which a reprogrammed iPSC is obtained.

### CROSS-REFERENCE TO RELATED APPLICATIONS

This application claims priority to U.S. Provisional Application No. 63/046,976, filed Jul. 1, 2020, the contents of which are incorporated herein by reference in their entirety.

### DESCRIPTION OF THE TEXT FILE SUBMITTED ELECTRONICALLY

The contents of the text file submitted electronically herewith are incorporated by reference in their entirety: a computer readable format copy of the Sequence Listing (filename: ELVT_011_01WO_SeqList_ST25.txt, date recorded Jul. 1, 2021, file size ˜89 kilobytes).

### BACKGROUND

Induced pluripotent stem cells (iPSCs) have transformed drug discovery and healthcare. iPSCs are generated by reprogramming somatic cells back into an embryonic-like pluripotent state that enables the development of various human cell types needed for research and/or therapeutic purposes.

iPSCs are typically derived by introducing one or more reprogramming factors (e.g., Oct3/4, Klf4, Sox2, Nanog, Lin28, c-Myc, and/or L-Myc) into a somatic cell. Although reprogramming factors can be introduced into a cell using standard approaches, these approaches suffer from various drawbacks. For example, self-replicating RNA systems use RNA replicons that are able to self-replicate. The nature of such replicating vectors poses a risk of genome integration. mRNA-based reprogramming is laborious and involves multiple transfections of mRNA due to fast turnover of mRNA molecules. Exogenous mRNA is also immunogenic, which necessitates the use of immune evasion factors (e.g., inhibitors of interferon pathways) and/or modified nucleotides to minimize toxicity.

Accordingly, there is a need in the art for improved compositions and methods for producing iPSCs.

### BRIEF SUMMARY

Provided herein are circular RNAs (circRNAs) encoding one or more reprogramming factors (e.g., transcription factors). The reprogramming factors may be, for example, Oct3/4, Klf4, Sox2, Nanog, Lin28, c-Myc, and/or L-Myc. In some embodiments, the circular RNAs can be used to generate integration-free iPSCs. The iPSCs can be used, for example, to derive specialized cell therapies or to generate disease-relevant cell types for advancing research in drug discovery.

In some embodiments, a recombinant circular RNA comprises a protein-coding sequence, wherein the protein-coding sequence encodes at least one reprogramming factor, wherein the at least one reprogramming factor is Oct3/4, Klf4, Sox2, Nanog, Lin28, c-Myc, or L-Myc, or a fragment or variant thereof.

In some embodiments, a complex comprises a recombinant circular RNA described herein and a lipid nanoparticle (LNP).

In some embodiments, a vector comprises a nucleic acid encoding a recombinant circular RNA disclosed herein.

In some embodiments, a composition comprises a recombinant circular RNA, a the complex or a vector described herein.

In some embodiments, a composition comprises two or more of recombinant circular RNAs, wherein the recombinant circular RNAs encode reprogramming factors selected from those in Table 1, 2, or 3.

In some embodiments, a composition comprises two or more recombinant circular RNAs, wherein the composition comprises a combination of recombinant circular RNAs encoding the reprogramming factors selected from: (i) Oct3/4, Klf4, Sox2, and c-Myc; (ii) Oct3/4, Klf4, Sox2, and L-Myc; (iii) Oct3/4, Klf4, and Sox2; (iv) Oct3/4, Klf4, Sox2, Nanog, Lin28, and c-Myc; or (iv) Oct3/4, Klf4, Sox2, Nanog, Lin28, and L-Myc.

In some embodiments, a cell comprises a recombinant circular RNA, a complex, a vector, or a composition of described herein.

In some embodiments, a method of expressing a protein in a cell comprises contacting the cell with a circular RNA, a complex, a vector, or a composition described herein, and maintaining the cell under conditions under which the protein is expressed.

In some embodiments, a method of producing an induced pluripotent stem cell (iPSC) comprises contacting a somatic cell with at least one recombinant circular RNA(s), a complex, a vector, and/or a composition described herein, and maintaining the cell under conditions under which a reprogrammed iPSC is obtained.

In some embodiments, a method of producing an induced pluripotent stem cell (iPSC) comprises contacting a CD34+ cell in suspension with at least one recombinant circular RNA(s), a complex, a vector, and/or a composition described herein, and maintaining the cell under conditions under which a reprogrammed iPSC is obtained.

In some embodiments, a method for reprogramming a cell comprises contacting a cell with one or more of: (i) a circular RNA encoding a reprogramming factor; (ii) a circular RNA that does not encode any protein or miRNA; (iii) a circular or linear RNA encoding a miRNA; and/or (iv) a circular or linear RNA encoding a viral protein.

In some embodiments, a method for reprogramming a cell comprises contacting a cell with each of: (i) a circular RNA encoding a reprogramming factor; (ii) a circular RNA that does not encode any protein or miRNA; (iii) a circular or linear RNA encoding a miRNA; and (iv) a circular or linear RNA encoding a viral protein.

In some embodiments, a method for reprogramming a cell comprises contacting a cell with each of: (i) a circular RNA encoding a reprogramming factor; (ii) a circular or linear RNA encoding a miRNA; and (iii) a circular or linear RNA encoding a viral protein.

In some embodiments, a method for reprogramming a cell comprises contacting a cell with each of: (i) a circular RNA encoding a reprogramming factor; and (ii) a circular or linear RNA encoding a miRNA.

In some embodiments, a method of increasing duration of protein expression in a cell comprises contacting a cell with a circular RNA, a complex, a vector, or a composition described herein, and maintaining the cell under conditions under which the protein is expressed, and wherein the duration of protein expression is increased relative to transfection of the cell with a linear RNA encoding the same protein.

In some embodiments, a method of improving cellular reprogramming efficiency comprises contacting a cell with a circular RNA, a complex, a vector, or a composition described herein, and maintaining the cell under conditions under which the protein is expressed, wherein the efficacy of cellular reprogramming is increased relative to a cellular reprogramming method in which linear RNA is used.

In some embodiments, a method of increasing the number of reprogrammed cell colonies formed after reprogramming comprises contacting a cell with a circular RNA, a complex, a vector, or a composition described herein, and maintaining the cell under conditions under which the protein is expressed, wherein the number of reprogrammed cell colonies formed after reprogramming is increased relative to a cellular reprogramming method in which linear RNA is used.

In some embodiments, a method of reprogramming cells in suspension comprises contacting a cell in suspension with a circular RNA, a complex, a vector, or a composition described herein, and maintaining the cell under conditions under which the protein is expressed.

In some embodiments, a method of improving morphological maturation of reprogrammed colonies comprises contacting a cell in suspension with a circular RNA, a complex, a vector, or a composition described herein, and maintaining the cell under conditions under which the protein is expressed, wherein the morphological maturation is improved relative to a cellular reprogramming method in which linear RNA is used.

In some embodiments, a method of reprogramming a cell which produces reduced cell death as compared to a method using linear RNA comprises contacting a cell with a circular RNA, a complex, a vector, or a composition described herein, and maintaining the cell under conditions under which the protein is expressed.

In some embodiments, a method of reducing time from reprogramming to picking (manual selection of iPSC colonies by mechanical dissociation) comprises contacting a cell with a circular RNA, a complex, a vector, or a composition described herein, and maintaining the cell under conditions under which the protein is expressed, wherein the time is reduced relative to a reprogramming method using linear RNA.

In some embodiments, a method of reducing the number of transfections induce to effect reprogramming of a cell comprises contacting a cell with a circular RNA, a complex, a vector, or a composition described herein, and maintaining the cell under conditions under which the protein is expressed, relative to a method using linear RNA.

In some embodiments, a suspension culture comprises one or more CD34-expressing cells, wherein the CD34-expressing cells comprise one or more exogenous circRNAs encoding a reprogramming factor.

Also provided herein are circular RNAs encoding one or more transdifferentiation factors. The transdifferentiation factors may be, for example, one or more of the factors listed in Table 6. The circular RNAs encoding one or more transdifferentiation factors may be used to convert a first somatic cell type to a second somatic cell type.

In some embodiments, a method of directly converting a cell from a first cell type to a second cell type comprises contacting the cell with a recombinant circular RNA, a complex, a vector, and/or a composition described herein, and maintaining the cell under conditions under which the cell is converted to the second cell type.

In some embodiments, a method for reprogramming and editing the genome of a cell comprises contacting the cell with: (i) a recombinant circular RNA comprising a protein-coding sequence, wherein the protein-coding sequence encodes at least one reprogramming factor, and (ii) an enzyme capable of editing the DNA or RNA of the cell, or a nucleic acid encoding the same.

In some embodiments, a method for transdifferentiating and editing the genome of a cell comprises contacting the cell with: (i) a recombinant circular RNA comprising a protein-coding sequence, wherein the protein-coding sequence encodes at least one transdifferentiation factor, and (ii) an enzyme capable of editing the DNA or RNA of the cell, or a nucleic acid encoding the same.

In some embodiments, a composition comprises a somatic cell that comprises one or more exogenous circular RNAs encoding a reprogramming factor.

In some embodiments, a composition comprises a transdifferentiated cell, wherein the transdifferentiated cell comprises one or more exogenous circular RNAs encoding a transdifferentiation factor.

In some embodiments, a method for inducing a mesenchymal-to-epithelial transition (MET) of a somatic cell to an iPSC comprises contacting the somatic cell with one or more circular RNA encoding a reprogramming factor.

In some embodiments, a method for transdifferentiating a cell comprises contacting the cell with a recombinant circular RNA comprising a protein-coding sequence, wherein the protein-coding sequence encodes at least one transdifferentiation factor.

In some embodiments, a kit comprises a recombinant circular RNA, a complex, a vector, or a composition described herein.

In some embodiments, a kit comprises: (i) a vessel comprising a circular RNA encoding OCT4 and a buffer; (ii) a vessel comprising a circular RNA encoding SOX2 and a buffer; (iii) a vessel comprising a cirRNA encoding KLF4 and a buffer; and (iv) packaging and instructions therefor.

Also provided herein is a cell produced using one or more of the methods disclosed herein.

Also provided is an iPSC produced using one or more of the methods disclosed herein.

Also provided herein is a differentiated cell derived from an iPSC produced using one or more of the methods disclosed herein.

Other objects, advantages and features of the present invention will become apparent from the following specification.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is a schematic showing an exemplary protocol for circularizing linear RNA generated using chemical synthesis or in vitro transcription (IVT) to generate circular RNAs. First, linear RNA is prepared. The 5′ end of the linear RNA is then phosphorylated by amplification using primers specific to the flanking sequence. The 5′ and 3′ ends are subsequently ligated using T4 RNA ligase. The circular RNA is purified, or linear side products are denatured enzymatically. The circular RNA my then be contacted with (e.g., transfected into) cells and/or conjugated to a lipid nanoparticle.

FIG. 2A-2G is a schematic showing exemplary methods for circularizing linear RNA, including enzymatic ligation of a 5′ phosphate with a 3′-OH terminus (FIG. 2A), chemical ligation of a phosphate with OH-terminus (the 5′ or the 3′ end can be phosphorylated) (FIG. 2B); chemical ligation of a 3′ thiophosphate with a tosylated 5′ end (FIG. 2C); chemical ligation of a 3′-thiophosphate with a iodinated 5′-end (FIG. 2D); chemical ligation of a 3′-aldehyde with a 50 oxoamine (oxime circularization) (FIG. 2E); chemical ligation of a 5′- or 3′-azide with a 3′- or 5′-alkyne (Click circularization) (FIG. 2F); circularization by metal chelation (M=Zn²⁺ or Fe²⁺, (=terpyridine)) (FIG. 2G).

FIG. 3 is a schematic showing an illustrative method for circularizing linear RNA. In the intron-exon construct shown, a group I catalytic intron of the T4 phage Td gene is bisected in such a way to preserve structural elements critical for ribozyme folding. Exon fragment 2 (E2) is then ligated upstream of exon fragment 1 (E1), and a coding region roughly 1.1 kb in length is inserted between the exon-exon junction. During splicing, the 3′ hydroxyl group of a guanosine nucleotide engages in a transesterification reaction at the 5′ splice site, resulting in circularization of the intervening region and excision of the 3′ intron.

FIG. 4 illustrates permuted-intron exon (PIE)-based circRNA construct design and production of circRNA.

FIG. 5A-FIG. 5B illustrate nicked circular RNA. FIG. 5A shows an illustration of a circular RNA, and FIG. 5B shows the expected nicked RNAs resulting from nicking at each of three nicking sites indicated by the white triangles in “A.” The degradation products shown in B are illustrative, as nicking could occur anywhere along the length of the circRNA.

FIG. 6 shows agarose gel electrophoresis of in vitro transcription products from a DNA template corresponding to either a full-length (WT) or truncated (ΔSS) permuted intron-exon (PIE) precursor RNA.

FIG. 7 shows splice junction-specific RT-PCR results to verify that the circRNA band contains circularized RNA.

FIG. 8A shows the distribution of RNA species remaining after each indicated step for each of the six reprogramming factors. FIG. 8B shows results of RNase R Digestion of circRNA preparations.

FIG. 9A-FIG. 9F show results from reprogramming of fibroblasts using linear and circular RNA. FIG. 9A shows a timeline for reprogramming HDFs using linear and circular RNA. FIG. 9B shows expression levels of nuclear GFP (nGFP) protein encoded by linear or circ-encoded nGFP RNA spiked into the reprogramming cocktails as shown (Stemgent linear RNA or TriLink linear RNA or circRNA). Graph shows nGFP expression normalized as the percentage of the peak expression. FIG. 9C shows representative images showing the morphological transition from fibroblasts to iPSCs during RNA reprogramming. FIG. 9D shows whole well images of day 18 reprogrammed iPSC colonies expressing Tra-1-81, a pluripotency marker. FIG. 9E shows representative images of circRNA reprogrammed iPSCs. FIG. 9F shows confluency of iPSC colonies, as a quantification of iPSC reprogramming shown in FIG. 9D.

FIG. 10A-FIG. 10C provide data illustrating physical characteristic of iPSCs reprogrammed according to methods described herein. FIG. 10A shows representative images of iPSCs derived from Stemgent mRNA reprogramming kit (top), linear mRNA synthesized by Trilink (middle), and circRNA (bottom), from cultures between passage 3 and 5. FIG. 10B shows population doubling time (PDT) for iPSCs derived from RNA reprogramming, including 5 clones derived from circRNA, 2 clones derived from Stemgent kit, and 3 clones derived from Trilink linear mRNA. FIG. 10C shows SSEA expression in iPSC clones derived from RNA reprogramming as determined by flow cytometry. S=Stemgent mRNA kit-derived; L=Trilink linear mRNA-derived; C=circRNA-derived.

FIG. 11 shows the transfection schedule for the iPSC reprogramming experiments in Example 6.

FIG. 12A-FIG. 12D show morphological progression during reprogramming. FIG. 12A—4 Tx +EKB group. FIG. 12B—4 Tx −EKB group. FIG. 12C—2 Tx group. FIG. 12D—1 Tx group. Tx=transfection.

FIG. 13 shows cell culture images on Day 6 to assess cell toxicity resulting from the indicated transfection conditions.

FIG. 14A shows Tra-1-81 and Oct4 costaining of cell culture wells to assess iPSC reprogramming. FIG. 14B shows quantification of iPSC reprogramming shown in FIG. 14A.

FIG. 15A-FIG. 15D illustrate the results of muscle cell differentiation from fibroblasts using linear (TriLink) or circRNA encoding MyoD. FIG. 15A shows MyoD expression in mock, circRNA or linear mRNA-transfected cells. FIG. 15B shows myotube formation in mock, circRNA or linear mRNA-transfected cells. FIG. 15C shows expression of muscle-specific markers (myogenin, desmin, and myosin heavy chain (MHC)) in fibroblasts transfected with circRNA encoding MyoD. FIG. 15D shows myogenin, desmin, and myosin heavy chain (MHC) expression in fibroblasts transfected with linear mRNA MyoD.

FIG. 16A-16B illustrate validation of protein expression of gene of interest encoded by linear mRNA (TriLink) or circRNA. Images were acquired using a 20× objective. Scale bar=100 μM.

FIG. 17A-17C illustrate quantification of myogenic conversion and myotube formation in human dermal fibroblasts with linear mRNA vs. circRNA. FIG. 17A shows the fusion index, which is the ratio of nuclei (DAPI-positive) within Desmin-positive myotubes vs. total number of nuclei in the population. FIG. 17B shows percent overlap of MYOG-positive nucleic with Desmin-positive myotubes. FIG. 17C shows percent overlap between the muscle-specific marker myosin heavy chain (MHC) and Desmin-positive myotubes.

### DETAILED DESCRIPTION

Unless otherwise defined, all technical and scientific terms used herein have the same meaning as commonly understood by one of ordinary skill in the art to which this disclosure belongs. The terminology used in the detailed description herein is for the purpose of describing particular embodiments only and is not intended to be limiting.

Unless the context indicates otherwise, it is specifically intended that the various features described herein can be used in any combination. Moreover, in some embodiments, any feature or combination of features set forth herein can be excluded or omitted. To illustrate further, if, for example, the specification indicates that a particular amino acid can be A, G, I, L and/or V, this language also indicates that the amino acid can be any subset of these amino acid(s) for example A, G, I or L; A, G, I or V; A or G; only L; etc., as if each such subcombination is expressly set forth herein. Moreover, such language also indicates that one or more of the specified amino acids can be disclaimed. For example, in some embodiments the amino acid is not A, G or I; is not A; is not G or V; etc., as if each such possible disclaimer is expressly set forth herein.

All publications, patent applications, patents, GenBank or other accession numbers and other references mentioned herein are incorporated by reference in their entirety for all purposes.

### General Methods

The practice of the present invention will employ, unless otherwise indicated, conventional techniques of cell culturing, molecular biology (including recombinant techniques), microbiology, cell biology, biochemistry and immunology, which are within the skill of the art. Such techniques are explained fully in the literature, such as, Molecular Cloning: A Laboratory Manual, third edition (Sambrook et al., 2001) Cold Spring Harbor Press; Oligonucleotide Synthesis (P. Herdewijn, ed., 2004); Animal Cell Culture (R. I. Freshney), ed., 1987); Methods in Enzymology (Academic Press, Inc.); Handbook of Experimental Immunology (D. M. Weir & C. C. Blackwell, eds.); Gene Transfer Vectors for Mammalian Cells (J. M. Miller & M. P. Calos, eds., 1987); Current Protocols in Molecular Biology (F. M. Ausubel et al., eds., 1987); PCR: The Polymerase Chain Reaction, (Mullis et al., eds., 1994); Current Protocols in Immunology (J. E. Coligan et al., eds., 1991); Short Protocols in Molecular Biology (Wiley and Sons, 1999); Manual of Clinical Laboratory Immunology (B. Detrick, N. R. Rose, and J. D. Folds eds., 2006); Immunochemical Protocols (J. Pound, ed., 2003); Lab Manual in Biochemistry: Immunology and Biotechnology (A. Nigam and A. Ayyagari, eds. 2007); Immunology Methods Manual: The Comprehensive Sourcebook of Techniques (Ivan Lefkovits, ed., 1996); Using Antibodies: A Laboratory Manual (E. Harlow and D. Lane, eds., 1988); and others.

### Definitions

The following terms are used in the description herein and the appended claims.

The singular forms “a,” “an” and “the” are intended to include the plural forms as well, unless the context clearly indicates otherwise.

The term “about” as used herein when referring to a measurable value such as an amount of the length of a polynucleotide or polypeptide, dose, time, temperature, and the like, is meant to encompass variations of ±20%, ±10%, ±5%, ±1%, ±0.5%, or even ±0.1% of the specified amount.

As used herein, “and/or” refers to and encompasses any and all possible combinations of one or more of the associated listed items, as well as the lack of combinations when interpreted in the alternative (“or”).

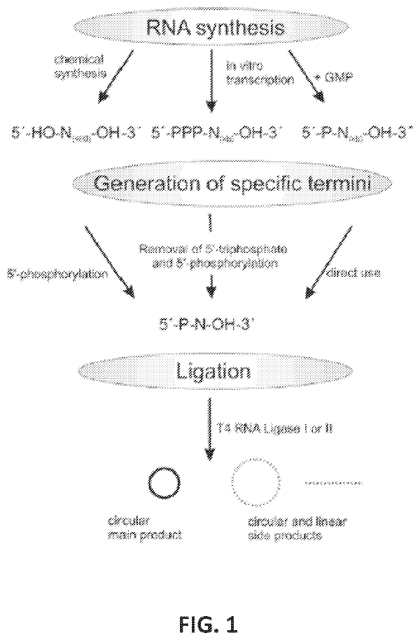

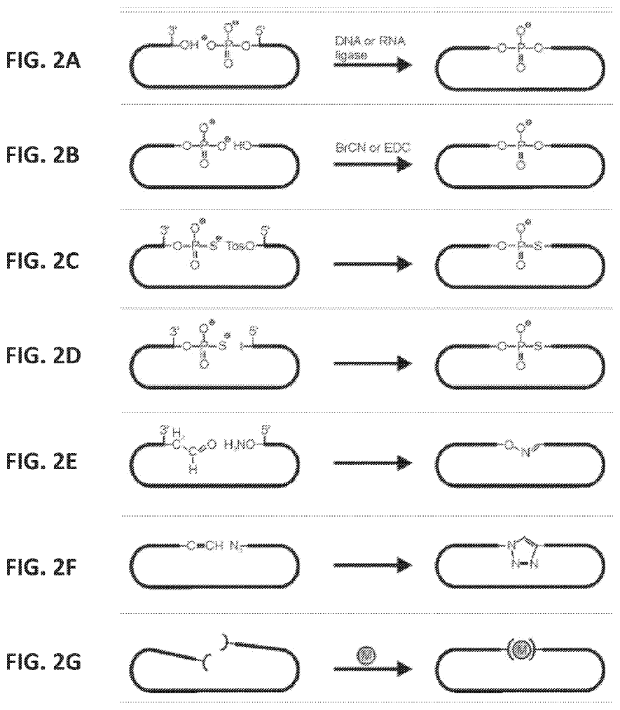

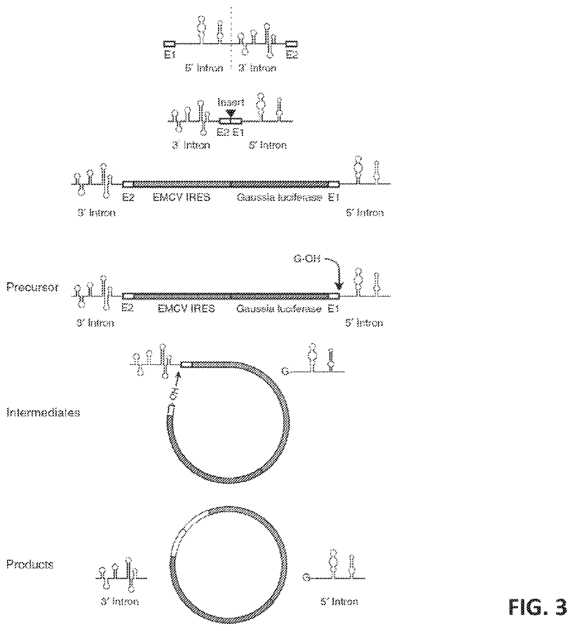

As used herein, “circular RNA” or “circRNA” refers to a type of single-stranded RNA which, unlike the better known linear RNA, forms a covalently closed continuous loop. Herein, any protein name preceded by “circ” refers to a circular RNA encoding that gene. RNAs may be circularized in a cell, by the cellular splicing machinery. For example, circular RNAs may be generated when the pre-mRNA splicing machinery “backsplices” to join a splice donor to an upstream splice acceptor, thereby producing a circular RNA that has covalently linked ends. Alternatively, circular RNAs may be generated in vitro, for example by circularization of a linear RNA produced by in vitro transcription (IVT). There are three general strategies for in vitro RNA circularization: chemical methods using cyanogen bromide or a similar condensing agent, enzymatic methods using RNA or DNA ligases (e.g., T4 RNA ligase I or II), and ribozymatic methods using self-splicing introns. A ribozymatic method utilizing a permuted group I catalytic intron is applicable for long RNA circularization and requires only the addition of GTP and Mg²⁺ as cofactors. This permuted intron-exon (PIE) splicing strategy consists of fused partial exons flanked by half-intron sequences. In vitro, these constructs undergo the double transesterification reactions characteristic of group I catalytic introns, but because the exons are already fused they are excised as covalently 5′ to 3′ linked circles (See FIG. 3). An illustrative protocol for circularizing linear RNA is provided in FIG. 1 and a list of illustrative linear RNA circularization strategies is provided in FIG. 2A-2G.

The terms “linear RNA” and “linear mRNA” are used interchangeably herein, as will be evident to a person of ordinary skill in the art based on context.

As used herein, “pluripotent” refers to a cell with the capacity, under different conditions, to differentiate to more than one differentiated cell type, and to differentiate to cell types characteristic of all three germ cell layers. In some embodiments, pluripotency may be evidenced by the expression of one or more pluripotent stem cell markers.

As used herein, the terms “induced pluripotent stem cells” and “iPSCs” refer to pluripotent cells that are generated from various differentiated (i.e., multipotent or non-pluripotent) somatic cells. iPSCs are substantially genetically identical to their respective differentiated somatic cell of origin and display characteristics similar to higher potency cells, such as embryonic stem (ES) cells, including the capacity to indefinitely self-renew in culture and the capacity to differentiate into other cell types. In some embodiments, iPSCs exhibit morphological (i.e., round shape, large nucleoli and scant cytoplasm) and growth properties (i.e., doubling time) akin to ES cells. In some embodiments, iPSCs express pluripotent cell-specific markers (e.g., Oct-4, SSEA-3, SSEA-4, Tra-1-60, Tra-1-81, but not SSEA-1).

As used herein, a “differentiated cell” or “somatic cell” is any cell that is not, in its native form, pluripotent as that term is defined herein. The term “somatic cell” also encompasses progenitor cells that are multipotent (e.g., can produce more than one cell type) but not pluripotent (e.g., can produce cells from all three germ layers).

The term “reprogramming” as used herein refers to a process of altering the differentiation state of a cell, such as a somatic cell, multipotent cell or progenitor cell. In some embodiments, reprogramming a cell may comprise converting a cell from a first cell type to a second cell type. In some embodiments, reprogramming may comprise altering the phenotype of a differentiated cell to a pluripotent phenotype. In some embodiments, reprogramming may refer to a process of “induced differentiation” or “transcription factor-directed differentiation” wherein an iPSC is converted into a differentiated cell.

As used herein, the term “reprogramming factor” refers to any factor or combination of factors that promotes the re-programming of a cell. A reprogramming factor may be, for example, a transcription factor. Illustrative reprogramming factors for producing iPSCs from differentiated cells include Oct3/4, Klf4, Sox2, Nanog, Lin28, c-Myc, and L-Myc. Illustrative reprogramming factors and combinations thereof for producing differentiated cells are provided in Table 6.

As used herein, “transdifferentiation” refers to a type of cellular reprogramming wherein one somatic cell type is directly converted into a second somatic cell type. In some embodiments, transdifferentiation may refer to “direct reprogramming” or “direct cell-fate conversion” wherein a somatic cell of a first cell type is converted into a somatic cell of a second cell type without going through an intermediate pluripotent state or progenitor cell type.

As used herein, “Internal ribosome entry site” or “IRES” is an RNA element that allows for initiation of translation in a cap-independent manner. An IRES may be, for example, a viral IRES or a mammalian IRES (e.g., a human IRES).

A “nucleotide triphosphate” or “NTP” is a molecule comprising a nitrogenous base bound to a 5-carbon sugar (either ribose or deoxyribose), with three phosphate groups bound to the sugar.

As used herein, a “modified NTP” is a NTP that has been chemically modified to confer favorable properties to a nucleic acid comprising the NTP. Such favorable properties may include, for example, reduced immunogenicity, increased stability, chemical functionality, or modified binding affinity.

The term “modified RNA” (e.g., “modified linear RNA” or “modified circular RNA”) is used to describe an RNA molecule which comprises one or more modified NTPs.

The term “vector” refers to a carrier for a nucleic acid (i.e., a DNA or RNA molecule), which can be used to introduce the nucleic acid into a cell. An “expression vector” is a vector that comprises a sequence encoding a protein or an RNA (e.g., a circular RNA) and the necessary regulatory regions needed for expression of the sequence in a cell. In some embodiments, the sequence encoding a protein or an RNA is operably linked to another sequence in the vector. The term “operably linked” means that the regulatory sequences necessary for expression of the sequence encoding a protein or an RNA are placed in the nucleic acid molecule in the appropriate positions relative to the sequence to effect expression of the protein or RNA.

As used herein, the terms “lipid nanoparticle” and “LNP” describe lipid-based particles in the submicron range. LNPs can have the structural characteristics of liposomes and/or may have alternative non-bilayer types of structures. LNPs may be conjugated to nucleic acids (e.g., DNA or RNA molecules) and used to deliver the nucleic acid to cells.

Methods of determining sequence similarity or identity between two or more nucleic acid sequences or amino acid sequences are known in the art. For example, sequence similarity or identity may be determined using the local sequence identity algorithm of Smith & Waterman, Adv. Appl. Math. 2, 482 (1981), by the sequence identity alignment algorithm of Needleman & Wunsch J Mol. Biol. 48, 443 (1970), by the search for similarity method of Pearson & Lipman, Proc. Natl. Acad. Sci. USA 85, 2444 (1988), by computerized implementations of these algorithms (GAP, BESTFIT, FASTA, and TFASTA in the Wisconsin Genetics Software Package, Genetics Computer Group, 575 Science Drive, Madison, WI), the Best Fit sequence program described by Devereux et al. Nucl. Acid Res. 12, 387-395 (1984), or by inspection.

Another suitable algorithm is the BLAST algorithm, described in Altschul et al. J. Mol. Biol. 215, 403-410, (1990) and Karlin et al. Proc. Natl. Acad. Sci. USA 90, 5873-5787 (1993). A particularly useful BLAST program is the WU-BLAST-2 program which was obtained from Altschul et al. Methods in Enzymology, 266, 460-480 (1996); blast.wustl/edu/blast/README.html. WU-BLAST-2 uses several search parameters, which are optionally set to the default values. The parameters are dynamic values and are established by the program itself depending upon the composition of the particular sequence and composition of the particular database against which the sequence of interest is being searched; however, the values may be adjusted to increase sensitivity. Further, an additional useful algorithm is gapped BLAST as reported by Altschul et al, (1997) Nucleic Acids Res. 25, 3389-3402. Unless otherwise indicated, percent identity is determined herein using the algorithm available at the internet address: blast.ncbi.nlm.nih.gov/Blast.cgi.

### Recombinant Circular RNAs

Provided herein are recombinant circular RNAs. In particular embodiments, the recombinant circular RNAs encode reprogramming factors that are (alone or in combination with other reprogramming factors) capable of reprogramming differentiated cells into iPSCs, capable of differentiating iPSCs into differentiated cells, and/or capable of differentiating one differentiated cell type into another differentiated cell type. For example, in some embodiments, the circular RNAs encode reprogramming factors for induced differentiation or transcription factor-directed differentiation.

In some embodiments, a recombinant circular RNA comprises from about 200 nucleotides to about 5,000 nucleotides. In some embodiments, the recombinant circular RNA comprises from about 200 to about 1,000 nucleotides. In some embodiments, the recombinant circular RNA comprises from about 1,000 nucleotides to about 2,500 nucleotides. In some embodiments, the circular RNA comprises from about 2,500 nucleotides to about 5,000 nucleotides. In some embodiments, the circular RNA comprises more than about 5,000 nucleotides.

In some embodiments, a recombinant circular RNA comprises one or more open reading frames. In some embodiments, a recombinant circular RNA comprises one or more protein-coding sequences. In some embodiments, a recombinant circular RNA does not comprise an open reading frame, and/or a protein-coding sequence.

In some embodiments, a recombinant circular RNA comprises a sequence encoding a reprogramming factor. In some embodiments, the reprogramming factor is a human or humanized reprogramming factor. In some embodiments, the reprogramming factor is a transcription factor.

In some embodiments, the reprogramming factor may be, for example, any one of the reprogramming factors listed in in Table 1. In some embodiments, the reprogramming factor is a fragment or variant of any one of the reprogramming factors listed in Table 1. In some embodiments, the reprogramming factor has at least 90%, at least 95%, or at least 99% sequence identity to any one of the reprogramming factors listed in Table 1.

TABLE 1
Reprogramming Factors

| | NCBI REFSEQ mRNA |
|---|---|
| Human Gene Symbol | Accession Number(s) |
| POU2F1 | NM_002697 |
| POU2F2 | NM_002698 |
| POU2F3 | NM_014352 |
| POU3F1 | NM_002699 |
| POU3F2 | NM_005604 |
| POU3F3 | NM_006236 |
| POU3F4 | NM_000307 |
| POU5F1 (encodes OCT4) | NM_002701, NM_203289 |
| POU5F2 | NM_153216 |
| POU6F1 | NM_002702, NR_026893 |
| POU6F2 | NM_007252 |
| SOX1 | NM_005986 |
| SOX2 | NM_003106 |
| SOX3 | NM_005634 |
| SOX4 | NM_003107 |
| SOX5 | NM_006940, NM_152989, |
| | NM_178010 |
| SOX6 | NM_017508, NM_033326, |
| | NM_001145811, |
| | NM_001145819 |
| SOX7 | NM_031439 |
| SOX8 | NM_014587 |
| SOX9 | NM_000346 |
| SOX10 | NM_006941 |
| SOX11 | NM_003108 |
| SOX12 | NM_006943 |
| SOX13 | NM_005686 |
| SOX14 | NM_004189 |
| SOX15 | NM_006942 |
| SOX17 | NM_022454 |
| SOX18 | NM_018419 |
| SOX21 | NM_007084 |
| SOX30 | NM_178424, NM_007017 |
| KLF1 | NM_006563 |
| KLF2 | NM_016270 |
| KLF3 | NM_016531 |
| KLF4 | NM_004235 |
| KLF5 | NM_001730 |
| KLF6 | NM_001300 |
| KLF7 | NM_003709 |
| KLF8 | NM_007250, NM_001159296 |
| KLF9 | NM_001206 |
| KLF10 | NM_005655, NM_001032282 |
| KLF11 | NM_003597 |
| KLF12 | NM_007249 |
| KLF13 | NM_015995 |
| KLF14 | NM_138693 |
| KLF15 | NM_014079 |
| KLF16 | NM_031918 |
| KLF17 | NM_173484 |
| POU5F1P1 | NR_002304 |
| MYC | NM_002467 |
| MYCL1 | NM_005376, NM_001033081, |
| | NM_001033082 |
| MYCN | NM_005378 |
| NANOG | NM_024865 |
| LIN28 | NM_024674 |
| THAP11 | NM_020457 |
| TERT | NM_198253, NM_198255 |
| MYOD1 | NM_002478 |
| ASCL1 | NM_004316 |
| SPI1 | NM_003120, NM_001080547 |
| CEBPA | NM_004364 |
| CEBPB | NM_005194 |
| NEUROG3 | NM_020999 |
| PDX1 | NM 000209 |
| MAFA | NM 201589 |
| ESRRB | NM_004452.2 |
| NKX3-1 | NM_006167 |
| GATA3 | NM_001002295 |

In some embodiments, the reprogramming factor is an RNA, such a micro RNA (miRNA). miRs such as the miRNA302(a-d) cluster and miR367 have been shown to improve the efficiency of reprogramming when used in conjunction with other reprogramming factors (See U.S. Pat. Nos. 8,791,248; 8,852,940; Poleganov et al., Human Gene Therapy. November 2015. 751-766). For example, the miRNA may be any one of the miRNA302 family (e.g., miR302d, miR302a, miR302c and miR302b) or miR367, or a fragment or variant thereof. In some embodiments, the reprogramming factor is any one of the following reprogramming factors, or a fragment or variant thereof: Oct4, Sox2, Klf4, c-Myc, Lin28, Nanog, Sall4, Utf1, p53, p21, p16^{Ink4a}, GLIS1, L-Myc, TGF-beta, MDM2, REM2, Cyclin D1, SV40 large T antigen, DOT1 L, CX43, MBD3, SIRT6, TCL1a, RARy, SNAIL, Lrh-1, or RCOR2.

In some embodiments, a recombinant circular RNA comprises a protein-coding sequence, wherein the protein-coding sequence encodes a reprogramming factor (e.g., a transcription factor). In some embodiments, the reprogramming factor is Oct3/4, Klf4, Sox2, Nanog, Lin28, c-Myc, and/or L-Myc, or a fragment or variant thereof. In some embodiments, the reprogramming factor is Oct3/4, Klf4, Sox2, Nanog, Lin28, and/or c-Myc, or a fragment or variant thereof. In some embodiments, the reprogramming factor is a human or a humanized reprogramming factor.

In some embodiments, a recombinant circular RNA encodes the reprogramming factor Oct3/4. In some embodiments, the encoded Oct3/4 has a sequence of SEQ ID NO: 1, or a sequence at least 90% or at least 95%. 96%, 97%, 98%, or 99% identical thereto. In some embodiments, the circular RNA encodes the reprogramming factor Oct3/4 and comprises or consists of the nucleic acid sequence of SEQ ID NO: 33. In some embodiments, the circular RNA encodes the reprogramming factor Oct3/4 and comprises a nucleic acid sequence that is at least 90% or at least 95%, 96%, 97%, 98%, or 99% identical to SEQ ID NO: 37.

In some embodiments, a recombinant circular RNA encodes the reprogramming factor Klf4. In some embodiments, the encoded Klf4 has the sequence of SEQ ID NO: 2 or 3, or a sequence at least 90% or at least 95%, 96%, 97%, 98%, or 99% identical thereto. In some embodiments, the circular RNA encodes the reprogramming factor Klf4 and comprises or consists of the nucleic acid sequence of SEQ ID NO: 37. In some embodiments, the circular RNA encodes the reprogramming factor Klf4 and comprises a nucleic acid sequence that is at least 90% or at least 95%, 96%, 97%, 98%, or 99% identical to SEQ ID NO: 37.

In some embodiments, a recombinant circular RNA encodes the reprogramming factor Sox2. In some embodiments, the Sox2 has the sequence of SEQ ID NO: 4, or a sequence at least 90% or at least 95%, 96%, 97%, 98%, or 99% identical thereto. In some embodiments, the circular RNA encodes the reprogramming factor Sox2 and comprises or consists of the nucleic acid sequence of SEQ ID NO: 34. In some embodiments, the circular RNA encodes the reprogramming factor Sox2 and comprises a nucleic acid sequence that is at least 90% or at least 95%. 96%, 97%, 98%, or 99% identical to SEQ ID NO: 34.

In some embodiments, a recombinant circular RNA encodes the reprogramming factor Nanog. In some embodiments, the Nanog has the sequence of SEQ ID NO: 5 or 6, or a sequence at least 90% or at least 95%, 96%, 97%, 98%, or 99% identical thereto. In some embodiments, the circular RNA encodes the reprogramming factor Nanog and comprises or consists of the nucleic acid sequence of SEQ ID NO: 36. In some embodiments, the circular RNA encodes the reprogramming factor Nanog and comprises a nucleic acid sequence that is at least 90% or at least 95%, 96%, 97%, 98%, or 99% identical to SEQ ID NO: 36.

In some embodiments, a recombinant circular RNA encodes the reprogramming factor Lin28. In some embodiments, the Lin28 has the sequence of SEQ ID NO: 7, or a sequence at least 90% or at least 95%, 96%, 97%, 98%, or 99% identical thereto. In some embodiments, the circular RNA encodes the reprogramming factor Lin28 and comprises or consists of the nucleic acid sequence of SEQ ID NO: 35. In some embodiments, the circular RNA encodes the reprogramming factor Lin28 and comprises a nucleic acid sequence that is at least 90% or at least 95%, 96%, 97%, 98%, or 99% identical to SEQ ID NO: 35.

In some embodiments, a recombinant circular RNA encodes the reprogramming factor c-Myc. In some embodiments, the c-Myc has the sequence of SEQ ID NO: 8 or 9, or a sequence at least 90% or at least 95%, 96%, 97%, 98%, or 99% identical thereto. In some embodiments, the circular RNA encodes the reprogramming factor c-Myc and comprises or consists of the nucleic acid sequence of SEQ ID NO: 38. In some embodiments, the circular RNA encodes the reprogramming factor c-Myc and comprises a nucleic acid sequence that is at least 90% or at least 95%, 96%, 97%, 98%, or 99% identical to SEQ ID NO: 38.

In some embodiments, a recombinant circular RNA encodes the reprogramming factor L-Myc. In some embodiments, the L-Myc has the sequence of any one of SEQ ID NO: 10-12, or a sequence at least 90% or at least 95%, 96%, 97%, 98%, or 99% identical thereto.

In some embodiments, the circular RNA encodes the reprogramming factor MyoD and comprises or consists of the nucleic acid sequence of SEQ ID NO: 32. In some embodiments, the circular RNA encodes the reprogramming factor MyoD and comprises a nucleic acid sequence that is at least 90% or at least 95%, 96%, 97%, 98%, or 99% identical to SEQ ID NO: 32.

In some embodiments, a recombinant circular RNA comprises two or more protein-coding nucleic acid sequences. For example, the recombinant circular RNA may comprise three, four, five, or six protein-coding sequences. In some embodiments, at least one of the protein-coding sequences encodes a reprogramming factor (e.g., a transcription factor).

In some embodiments, a recombinant circular RNA comprises two or more protein-coding sequences, wherein at least one of the protein-coding sequences encodes a reprogramming factor. In some embodiments, a recombinant circular RNA comprises two or more protein-coding sequences, wherein at least one of the protein-coding sequences encodes Oct3/4, Klf4, Sox2, Nanog, Lin28, c-Myc, or L-Myc, or fragments or variants thereof. In some embodiments, a recombinant circular RNA comprises two or more protein-coding sequences, wherein each of the protein-coding sequences are independently selected from Oct3/4, Klf4, Sox2, Nanog, Lin28, c-Myc, and L-Myc, or fragments or variants thereof.

In some embodiments, the present disclosure provides compositions of recombinant circular RNAs encoding reprogramming factors. In some embodiments, the composition further comprises a buffer. The buffer may comprise, for example, 1-10 mM sodium citrate. In some embodiments the pH of the buffer is about 2, about 2.5, about 3, about 3.5, about 4, about 4.5, about 5, about 5.5, about 6, about 6.5, about 7, about 7.5, about 8, about 8.5, about 9, about 9.5, about 10, about 10.5, about 11, about 11.5, or about 12. In some embodiments, the pH of the buffer is about 6.5.

In some embodiments, the composition comprises two or more recombinant circular RNAs, each encoding a reprogramming factor selected from Oct3/4, Klf4, Sox2, Nanog, Lin28, c-Myc, and L-Myc. In some embodiments, the composition comprises two or more recombinant circular RNAs, each encoding a reprogramming factor selected from the combinations provided in Table 2.

TABLE 2
Reprogramming factor combinations

| Combination Ref | Reprogramming Factor 1 | Reprogramming Factor 2 |
|---|---|---|
| A1 | Oct3/4 | Klf4 |
| A2 | Oct3/4 | Sox2 |
| A3 | Oct3/4 | Nanog |
| A4 | Oct3/4 | Lin28 |
| A5 | Oct3/4 | c-Myc |
| A6 | Oct3/4 | L-Myc |
| A7 | Klf4 | Sox2 |
| A8 | Klf4 | Nanog |
| A9 | Klf4 | Lin28 |
| A10 | Klf4 | c-Myc |
| A11 | Klf4 | L-Myc |
| A12 | Sox2 | Nanog |
| A13 | Sox2 | Lin28 |
| A14 | Sox2 | c-Myc |
| A15 | Sox2 | L-Myc |
| A16 | Nanog | Lin28 |
| A17 | Nanog | c-Myc |
| A18 | Nanog | L-Myc |
| A19 | Lin28 | c-Myc |
| A20 | Lin28 | L-Myc |
| A21 | c-Myc | L-Myc |

In embodiments wherein the recombinant circular RNA comprises more than one protein-coding nucleic acid sequence, each sequence may be separated by a sequence encoding a self-cleaving peptide, such as a 2A peptide. Illustrative 2A peptides include, but are not limited to, EGRGSLLTCGDVEENPGP (SEQ ID NO: 17), ATNFSLLKQAGDVEENPGP (SEQ ID NO: 18), QCTNYALLKLAGDVESNPGP (SEQ ID NO: 19), and VKQTLNFDLLKLAGDVESNPGP (SEQ ID NO: 20). In some embodiments, each protein-coding nucleic acid sequence may be separated by an IRES.

In some embodiments, a recombinant circular RNA comprises a protein-coding sequence and a second sequence. In some embodiments, the protein-coding sequence encodes Oct3/4, Klf4, Sox2, Nanog, Lin28, c-Myc, or L-Myc, or fragments or variants thereof. In some embodiments, the second sequence is a sequence from one or more of circBIRC6, circCORO1C, or circMAN1A2. circBIRC6, circCORO1C and circMAN1A2 are endogenously expressed circRNAs and have been shown to be enriched in human ESCs and thought to act as a “miR sponge”. Thus, they may have a regulatory role in promoting pluripotency by counteracting certain miRNAs (e.g. miR34a and/or miR145) that are known to suppress expression of the pluripotency-associated transcription factors NANOG, SOX2 and OCT4 (Yu et al. Nat Commun 8, 1149 (2017)).

Circular RNAs lack a 5′ 7-methylguanosine cap structure which is required for efficient translation of linear mRNAs. For a circular RNA to be translated, therefore, an alternative mechanism of recruiting the ribosome may be used. For example, an internal ribosome entry site (IRES) may be used, which directly binds initiation factors or the ribosome itself. Accordingly, in some embodiments, a recombinant circular RNA comprises an internal ribosome entry site (IRES). In some embodiments, the IRES engages a eukaryotic ribosome. In some embodiments, the IRES is operatively linked to a protein-coding nucleic acid sequence.

Examples of IRES sequences include sequences derived from a wide variety of viruses, for example from leader sequences of picornavirus UTR's (such as the encephalomyocarditis virus (EMCV)), the polio leader sequence, the hepatitis A virus leader, the hepatitis C virus IRES, human rhinovirus type 2 IRES, an IRES element from the foot and mouth disease virus, a giardiavirus IRES, and the like. A variety of nonviral IRES sequences may also be used, including, but not limited to IRES sequences from yeast, as well as the human angiotensin II type 1 receptor IRES, fibroblast growth factor IRESs, vascular endothelial growth factor IRES, and insulin-like growth factor 2 IRES. Additional IRES sequences suitable for use in the recombinant circular RNAs described herein include those described in the database available at http://iresite.org/.

In some embodiments, the circular RNA comprises intronic elements that flank the protein coding sequence. Intronic elements may be backspliced by cellular splicing machinery to yield a circular RNA that is covalently closed. Accordingly, in some embodiments, a circular RNA comprises a first intronic element located 5′ to the protein coding sequence, and a second intronic element located 3′ to the protein coding sequence.

In some embodiments, a circular RNA is generated by circularizing a linear RNA. In some embodiments, a linear RNA may be self-circularizing, for example if it comprises self-splicing introns. Because circular RNAs do not have 5′ or 3′ ends, they may be resistant to exonuclease-mediated degradation and may be more stable than most linear RNAs in cells.

In some embodiments, the intronic elements are selected from any known intronic element(s), in any combination and in any multiples and/or ratios. Examples of intronic elements include those described in those described in the circBase circular RNA database (Glazar et al. RNA 20:1666-1670 (2014); and www.circbase.org) and in Rybak-Wolf et al. Mol. Cell 58(5):870-885 (2015), each of which are incorporated by reference herein in their entirety. In some embodiments, the intronic element is a mammalian intron or a fragment thereof. In some embodiments, the intronic element is a non-mammalian intron (e.g., a self-splicing group I intron, a self-splicing group II intron, a spliceosomal intron, or a tRNA intron), or a fragment thereof.

In some embodiments, the circular RNA comprises one or more additional elements which improves the stability of and/or enhances translation of the protein-encoding sequence from the circular RNA. For example, in some embodiments, the circular RNA may comprise a Kozak sequence. One example of a Kozak consensus sequence is: RCC(AUG)G (SEQ ID NO: 21), with the start codon in parentheses, and the “R” at position −3 representing a purine (A or G). Another example of a Kozak consensus sequence is RXY(AUG) (SEQ ID NO: 22), where R is a purine (A or G), Y is either C or G, and X is any base.

In some embodiments, a circular RNA comprises a first intronic element, a protein coding-sequence, and a second intronic element. In some embodiments, a circular RNA comprises an IRES and a protein-coding sequence. In some embodiments, a circular RNA comprises a first intronic sequence, an IRES, a protein-coding sequence, and a second intronic sequence.

In some embodiments, a circular RNA comprises a sequence encoding a reprogramming factor (e.g., a transcription factor). In some embodiments, a circular RNA comprises a first intronic element, a sequence encoding a reprogramming factor, and a second intronic element.

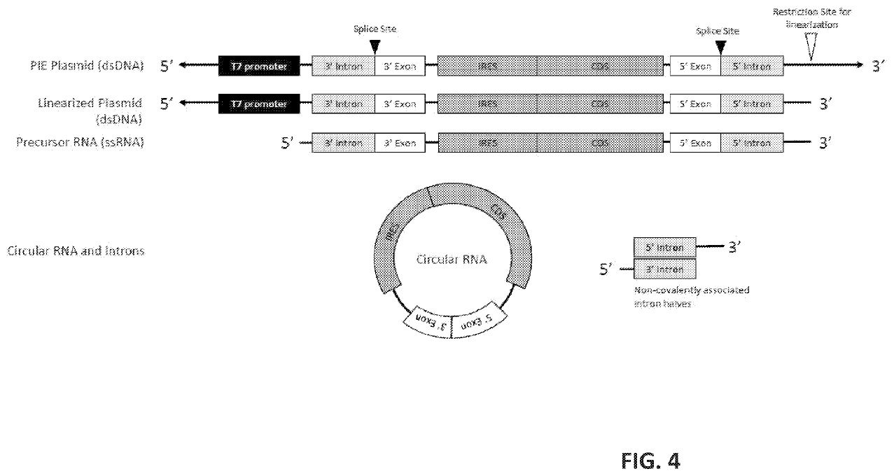

In some embodiments, a circular RNA comprises an IRES and a sequence encoding a reprogramming factor. In some embodiments, a circular RNA comprises a first intronic sequence, an IRES, a sequence encoding a reprogramming factor, and a second intronic sequence. In some embodiments, a circular RNA comprises an IRES and a sequence encoding a reprogramming factor. In some embodiments, a circular RNA comprises a first intronic element, an IRES, a sequence encoding a reprogramming factor, and a second intronic element. Exemplary schematics of the arrangement of elements in the circular RNAs are provided in FIG. 4. See also US 2020/0080106, which is incorporated herein by reference.

In some embodiments, a circular RNA comprises a sequence encoding Oct3/4, Klf4, Sox2, Nanog, Lin28, c-Myc, or L-Myc. In some embodiments, a circular RNA comprises a first intronic element, a sequence encoding Oct3/4, Klf4, Sox2, Nanog, Lin28, c-Myc, or L-Myc, and a second intronic element.

In some embodiments, a circular RNA comprises an IRES and a sequence encoding Oct3/4, Klf4, Sox2, Nanog, Lin28, c-Myc, or L-Myc. In some embodiments, a circular RNA comprises a first intronic sequence, an IRES, a sequence encoding Oct3/4, Klf4, Sox2, Nanog, Lin28, c-Myc, or L-Myc, and a second intronic sequence. In some embodiments, a circular RNA comprises an IRES and a sequence encoding Oct3/4, Klf4, Sox2, Nanog, Lin28, c-Myc, or L-Myc. In some embodiments, a circular RNA comprises a first intronic element, an IRES, a sequence encoding Oct3/4, Klf4, Sox2, Nanog, Lin28, c-Myc, or L-Myc, and a second intronic element.

Circular RNAs may also comprise modified bases and/or NTPs. In some embodiments, the recombinant circular RNAs comprise modified NTPs. In some embodiments, the recombinant circular RNAs are modified circular RNAs.

Modified bases include synthetic and natural bases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine and 3-deazaguanine and 3-deazaadenine. Further modified bases include tricyclic pyrimidines such as phenoxazine cytidine(1H-pyrimido[5,4-b][1,4]benzoxazin-2(3H)-one), phenothiazine cytidine (1H-pyrimido[5,4-b][1,4]benzothiazin-2(3H)-one), G-clamps such as a substituted phenoxazine cytidine (e.g. 9-(2-aminoethoxy)-H-pyrimido[5,4-b][1,4]benzoxazin-2(3H)-one), carbazole cytidine (2H-pyrimido[4,5-b]indol-2-one), pyridoindole cytidine (H-pyrido[3′,2′:4,5]pyrrolo[2,3-d]pyrimidin-2-one). Modified bases may also include those in which the purine or pyrimidine base is replaced with other heterocycles, for example 7-deaza-adenine, 7-deazaguanosine, 2-aminopyridine and 2-pyridone.

In some embodiments, the recombinant circular RNAs comprise modified backbones. Examples of modified RNA backbones include those that comprise phosphorothioates, chiral phosphorothioates, phosphorodithioates, phosphotriesters, aminoalkyl-phosphotriesters, methyl and other alkyl phosphonates including 3-alkylene phosphonates, 5′-alkylene phosphonates and chiral phosphonates, phosphinates, phosphoramidates including 3′-amino phosphoramidate and aminoalkyl-phosphoramidates, thionophosphoramidates, thionoalkylphosphonates, thionoalkyl-phosphotriesters, selenophosphates and boranophosphates having normal 3′-5′ linkages, 2′-5′ linked analogs of these, and those having inverted polarity wherein one or more internucleotide linkages is a 3′ to 3′, 5′ to 5′ or 2′ to 2′ linkage.

In some embodiments, the circular RNAs may be modified by chemically linking to the RNA one or more moieties or conjugates which enhance the activity, cellular distribution or cellular uptake. For example, a circular RNA may be conjugated to intercalators, reporter molecules, polyamines, polyamides, polyethylene glycols, polyethers, groups that enhance the pharmacodynamic properties of oligomers, or groups that enhance the pharmacokinetic properties of oligomers. In some embodiments, the circular RNAs may be conjugated to cholesterols, lipids, phospholipids, biotin, phenazine, folate, phenanthridine, anthraquinone, acridine, fluoresceins, rhodamines, coumarins, or dyes. Groups that enhance the pharmacodynamic properties, include groups that improve RNA uptake, enhance oligomer resistance to degradation, and/or strengthen sequence-specific hybridization with RNA. Groups that enhance the pharmacokinetic properties include groups that improve oligomer uptake, distribution, metabolism or excretion. The circular RNAs may also be conjugated to active drug substances, for example, aspirin, warfarin, phenylbutazone, ibuprofen, suprofen, fenbufen, ketoprofen, (S)-(+)-pranoprofen, carprofen, dansylsarcosine, 2,3,5-triiodobenzoic acid, flufenamic acid, folinic acid, a benzothiadiazide, chlorothiazide, a diazepine, indomethicin, a barbiturate, a cephalosporin, a sulfa drug, an antidiabetic, an antibacterial or an antibiotic. In some embodiments, a recombinant circular RNA is conjugated to a lipid nanoparticle (LNP).

In some embodiments, the circular RNA is part of a complex. In some embodiments, a complex comprises a recombinant circular RNA and a lipid nanoparticle (LNP). In some embodiments, the recombinant circular RNA and the LNP are conjugated. In some embodiments, the recombinant circular RNA and the LNP are covalently conjugated. In some embodiments, the recombinant circular RNA and the LNP are non-covalently conjugated.

The LNP may comprise, for example, one or more cationic lipids, non-cationic lipids, and/or PEG-modified lipids. In some embodiments, the LNP may comprise at least one of the following cationic lipids: C12-200, DLin-KC2-DMA, DODAP, HGT4003, ICE, HGT5000, or HGT5001. In some embodiments, the LNP comprises cholesterol and/or a PEG-modified lipid. In some embodiments, the LNP comprises DMG-PEG2K. In some embodiments, the LNP comprises one of the following: C12-200, DOPE, cholesterol, DMG-PEG2K; DODAP, DOPE, cholesterol, DMG-PEG2K; HGT5000, DOPE, cholesterol, DMG-PEG2K, HGT5001, DOPE, or DMG-PEG2K. In some embodiments, the LNP comprises polyethyleneimine (PEI).

In some embodiments, the recombinant circular RNA is substantially non-immunogenic. In some embodiments, a circular RNA is considered non-immunogenic if it does not induce the expression or activity of one or more interferon-regulated genes (e.g., one or more genes described at www.interferome.org). In some embodiments, the interferon-regulated genes are selected from IFN-alpha, IFN-beta, and/or TNF-alpha. Various modifications can be made to the circular RNA to reduce the immunogenicity thereof. For example, in some embodiments, the circular RNA may be modified to comprise one or more M-6-methyladenosine (m⁶A), 5-methylcytosine (5mC), or pseudouridine residues.

In some embodiments, the circular RNAs described herein are less immunogenic than linear RNA. For example, in some embodiments, a circular RNA does not substantially induce the expression and/or activity of one or more interferon-regulated genes. In some embodiments, a circular RNA induces the expression and/or activity of one or more interferon-regulated genes about 10%, about 20%, about 30%, about 40%, about 50%, about 60%, about 70%, about 80%, about 90%, or about 100% less than a linear RNA.

In some embodiments, the circular RNAs described herein have a longer cellular half-life than linear RNA. For example, a circular RNA may have a half-life that is about 10%, about 20%, about 30%, about 40%, about 50%, about 60%, about 70%, about 80%, about 90%, or about 100% longer than that of a linear RNA. In some embodiments, a circular RNA may have a half-life that is about 4 hours, about 12 hours, about 18 hours, about 24 hours, about 2 days, about 3 days, about 4 days, about 5 days, about 10 days, or about 10 days longer than that of a linear RNA.

In some embodiments, the recombinant circular RNAs don't replicate in the cells. In some embodiments, the recombinant circular RNAs are risk-free for genome integration.

Circular RNAs may be generated using in vitro transcription (IVT), according to standard protocols and/or by using commercially-available kits (e.g., the MAXIscript® or MEGAscript® kits from ThermoFisher®). For example, an illustrative IVT protocol uses a purified linear DNA template (i.e., a DNA molecule encoding a circular RNA as described herein), ribonucleotide triphosphates, a buffer system that includes DTT and magnesium ions, and an appropriate phage RNA polymerase to produce a circular RNA. The DNA template contains a double-stranded promoter region where the phage polymerase binds and initiates RNA synthesis. Reaction conditions (e.g., the type of nucleotide salt, type and concentration of salt in the transcription buffer, enzyme concentration and pH) are optimized for the particular polymerase used and for the entire set of components, in order to achieve optimal yields. Large-scale IVT reactions can produce up to 120-180 μg RNA per microgram template in a 20 μl reaction. In some embodiments, circular RNAs may be generated using RNA synthesis, according to standard protocols.

Various methods for circularizing RNAs are known in the art. For example, an illustrative protocol for circularizing linear RNA is provided in FIG. 1 and a list of illustrative linear RNA circularization strategies is provided in FIG. 2A-2G. In some embodiments, a RNA is self-circularizing, for example, if it contains self-splicing introns.

Also provided herein are nucleic acids (i.e., DNA molecules) encoding the circular RNAs described herein, and vectors comprising the same.

### Methods for Expressing a Protein (e.g., a Reprogramming Factor) in a Cell Using Circular RNA

Provided herein are methods for expressing a protein in a cell, wherein the protein is encoded by a circular RNA. In some embodiments, the protein is a reprogramming factor. In some embodiments, the reprogramming factor is a transcription factor. In some embodiments, the protein is Oct3/4, Klf4, Sox2, Nanog, Lin28, c-Myc, and/or L-Myc. In some embodiments, the protein is Oct3/4, Klf4, Sox2, Nanog, Lin28, and/or c-Myc.

In some embodiments, a method for expressing a protein in a cell comprises contacting the cell with at least one of the recombinant circular RNAs, vectors, complexes, or compositions described herein, and maintaining the cell under conditions under which the protein is expressed.

In some embodiments, a method for expressing a protein in a cell comprises contacting the cell with a first circular RNA and at least one additional circular RNA and maintaining the cell under conditions under which the protein is expressed. In some embodiments, a method for expressing a protein in a cell comprises contacting the cell with a first circular RNA and a second circular RNA and maintaining the cell under conditions under which the protein is expressed. In some embodiments, a method for expressing a protein in a cell comprises contacting the cell with a first, second, and third circular RNA, and maintaining the cell under conditions under which the protein is expressed. In some embodiments, a method for expressing a protein in a cell comprises contacting the cell with at least four circular RNAs, at least five circular RNAs, at least six circular RNAs, at least seven circular RNAs, at least eight circular RNAs, at least nine circular RNAs, or at least ten circular RNAs, and maintaining the cell under conditions under which the protein is expressed.

In some embodiments, a method for expressing a protein in a cell comprises contacting the cell with a first circular RNA encoding Oct3/4, Klf4, Sox2, Nanog, Lin28, c-Myc, or L-Myc and at least one additional circular RNA, and maintaining the cell under conditions under which the protein is expressed. In some embodiments, a method for expressing a protein in a cell comprises contacting the cell with a first circular RNA encoding Oct3/4, Klf4, Sox2, Nanog, Lin28, c-Myc, or L-Myc and at least two, at least three, at least four, at least five, at least six, at least seven, at least eight, at least nine, or at least ten additional circular RNAs, and maintaining the cell under conditions under which the protein is expressed. In some embodiments, a method for expressing a protein in a cell comprises contacting the cell with multiple circular RNAs (e.g., at least two, at least four, at least five, at least six, at least seven, at least eight, at least nine, or at least ten) circular RNAs, wherein each circular RNA encodes Oct3/4, Klf4, Sox2, Nanog, Lin28, c-Myc, or L-Myc, and maintaining the cell under conditions under which the protein is expressed.

In some embodiments, a method for expressing a protein in a cell comprises contacting the cell with (i) a first circular RNA encoding Oct3/4, Klf4, Sox2, Nanog, Lin28, c-Myc, or L-Myc and, (ii) at least one additional circular RNA, wherein the at least one additional circular RNA is circBIRC6, circCORO1C, or circMAN1A2, and maintaining the cell under conditions under which the protein is expressed. In some embodiments, the additional circular RNA is circBIRC6. In some embodiments, circBIRC6 has a sequence of SEQ ID NO: 13, or a sequence at least 90% or at least 95% identical thereto. In some embodiments, the additional circular RNA is circCORO1C. In some embodiments, circCORO1C has a sequence of SEQ ID NO: 14, or a sequence at least 90% or at least 95% identical thereto. In some embodiments, the additional circular RNA is circMAN1A2. In some embodiments, the circMAN1A2 has a sequence of SEQ ID NO: 15, or a sequence at least 90% or at least 95% identical thereto.

In some embodiments, a method for expressing a protein in a cell comprises contacting the cell with circular RNAs each encoding one of Oct4, Sox2, Klf4, and cMyc. In some embodiments, a method for expressing a protein in a cell comprises contacting the cell with circular RNAs each encoding one of Oct4, Sox2, Klf4, cMyc, and Lin28. In some embodiments a method for expressing a protein in a cell comprises contacting the cell with (i) circular RNAs each encoding one of Oct4, Sox2, Klf4, cMyc, and Lin28, and (ii) circBIRC6, circCORO1C, and circMAN1A2.

In some embodiments, the cell is a prokaryotic cell. In some embodiments, the cell is a eukaryotic cell. In some embodiments, the cell is an animal cell. In some embodiments, the cell is a mammalian cell (e.g., a murine, bovine, simian, porcine, equine, ovine, or human cell). In some embodiments, the cell is a human cell. In some embodiments, the cell is a yeast, fungi, or plant cell.

In some embodiments, the cell is a somatic cell. In some embodiments, the cell is a fibroblast, a peripheral blood-derived cell, an endothelial progenitor cell, a cord-blood derived cell, a hepatocyte, a keratinocyte, a melanocyte, an adipose-tissue derived cell, or a urine-derived cell (e.g., a renal epithelial progenitor cell). In some embodiments, the cell is an epithelial cell, an endothelial cell, a neuronal cell, an adipose cell, a cardiac cell, a skeletal muscle cell, an immune cell, a hepatic cell, a splenic cell, a lung cell, a circulating blood cell, a gastrointestinal cell, a renal cell, a bone marrow cell, a progenitor cell, or a pancreatic cell. In some embodiments, the cell is isolated from any somatic tissue including, but not limited to brain, liver, lung, gut, stomach, intestine, fat, muscle, uterus, skin, spleen, endocrine organ, bone, etc.

In some embodiments, the cell is an adherent cell. In some embodiments, the cell is a non-adherent cell (e.g., a suspension cell such as a CD34+ cell).

In some embodiments, the cell is contacted once with a circular RNA. In some embodiments the cell is contacted with the circular RNA more than once (e.g., 2, 3, 4, 5, 6, 7, 8, 9, or 10 times). In some embodiments, the contacting is performed at effective intervals. The effective intervals may be, for example, once per day, once every other day, once every three days, once per week, once every two weeks, or once per month.

In some embodiments, the contacting comprises transfecting a circular RNA, or a vector comprising a nucleic acid (i.e., a DNA molecule) encoding the same, into the cell. In some embodiments, the circular RNA is transfected into the cell using lipid-mediated transfection. Lipid-mediated transfection stimulates active uptake of nucleic acids by endocytosis. An exemplary lipid-mediated transfection reagent is Lipofectamine® (e.g., Lipofectamine® RNAiMAX®, from ThermoFisher®). In some embodiments, a method for transfecting a cell comprises the steps of (i) diluting the RNA or DNA and the transfection reagent in separate tubes, (ii) combining the DNA or RNA with the transfection reagent to form complexes, (iii) adding the complexes to the cells, (iv) assaying the cells for protein expression. Detection of protein expression in cells can be achieved by several techniques including Western blot analysis, immunocytochemistry, and fluorescence-mediated detection (e.g., FACS), among others.

In some embodiments, the contacting comprises electroporating a circular RNA, or a vector comprising a nucleic acid (i.e., a DNA molecule) encoding the same, into the cell. Electroporation delivers nucleic acids by transiently opening holes in the cell membrane while the cell is in a solution in which the nucleic acid is present at high concentration.

In some embodiments, the contacting comprises incubating the cells with circRNA-LNP complexes.

In some embodiments, the contacting comprises one or more techniques such as ballistic transfection (i.e., gene gun or biolistic transfection), magnetofection, peptide-mediated transfection (either non-covalent peptide/RNA nanoparticle-based transfection such as the N-TER™ Transfection System from Sigma-Aldrich or by covalent attachment of the peptide to the RNA), and/or microinjection. Combinations of these techniques used in succession or simultaneously can also be used.

As explained above, the methods for expressing a protein in a cell may comprise maintaining the cell under conditions under which the protein is expressed. Such conditions are well known to those of skill in the art and may vary by cell type. For example, in some embodiments, the cell may be maintained in normal culture media (with or without serum), at about 37° C. in an atmosphere comprising about 5% CO₂.

Methods for Producing iPSCs

Also provided herein are methods for reprogramming somatic cells and methods for producing iPSCs. In some embodiments, a method of producing an iPSC comprises contacting a somatic cell with at least one of the recombinant circular RNAs, complexes, vectors, or compositions described herein, and maintaining the cell under conditions under which a reprogrammed iPSC is obtained.

In some embodiments, a method of producing an iPSC comprises contacting a somatic cell with at least one circular RNA encoding a reprogramming factor (e.g., a transcription factor), and maintaining the cell under conditions under which a reprogrammed iPSC is obtained. The reprogramming factor may be, for example, any of the reprogramming factors shown in Table 1. In some embodiments, the reprogramming factor is Oct3/4, Klf4, Sox2, Nanog, Lin28, c-Myc, or L-Myc. In some embodiments, the reprogramming factor is Oct3/4. In some embodiments, the reprogramming factor is Klf4. In some embodiments, the reprogramming factor is Sox2. In some embodiments, the reprogramming factor is Nanog. In some embodiments, the reprogramming factor is Lin28. In some embodiments, the reprogramming factor is c-Myc. In some embodiments, the reprogramming factor is L-Myc.

In some embodiments, a method of producing an iPSC comprises contacting a somatic cell with more than one circular RNA, wherein each circular RNA encodes a reprogramming factor, and maintaining the cell under conditions under which a reprogrammed iPSC is obtained. In some embodiments, the cell is contacted with at least 2, at least 3, at least 4, at least 5, at least 6, at least 7, at least 8, at least 9, at least 10, or more circular RNAs, each encoding a reprogramming factor. In some embodiments, a method of producing an iPSC comprises contacting a somatic cell with 6 circular RNAs encoding the reprogramming factors Oct3/4, Klf4, Sox2, Nanog, Lin28, and c-Myc. In some embodiments, a method of producing an iPSC comprises contacting a somatic cell with 4 circular RNAs encoding the reprogramming factors Oct3/4, Klf4, Sox2, and c-Myc. In some embodiments, a method of producing an iPSC comprises contacting a somatic cell with 4 circular RNAs encoding the reprogramming factors Oct3/4, Klf4, Sox2, and L-Myc. In some embodiments, a method of producing an iPSC comprises contacting a somatic cell with 6 circular RNAs encoding the reprogramming factors Oct3/4, Klf4, Sox2, Nanog, Lin28, and L-Myc. In some embodiments, a method of producing an iPSC comprises contacting a somatic cell with 5 circular RNAs encoding the reprogramming factors Oct3/4, Klf4, Sox2, Lin28, and c-Myc. In some embodiments, a method of producing an iPSC comprises contacting a somatic cell with 5 circular RNAs encoding the reprogramming factors Oct3/4, Klf4, Sox2, Lin28, and L-Myc.

In some embodiments, a method of producing an iPSC comprises contacting a somatic cell with two circular RNAs and maintaining the cell under conditions under which a reprogrammed iPSC is obtained. In some embodiments, the first and second circular RNAs each encode a reprogramming factor selected from Oct3/4, Klf4, Sox2, Nanog, Lin28, c-Myc, and L-Myc, wherein the first and second circular RNAs do not encode the same reprogramming factor. In some embodiments, the first circular RNA encodes Oct3/4 and the second circular RNA encodes Sox2.

In some embodiments, a method of producing an iPSC comprises contacting a somatic cell with three circular RNAs and maintaining the cell under conditions under which a reprogrammed iPSC is obtained. In some embodiments, the first, second, and third circular RNAs each encode a reprogramming factor selected from Oct3/4, Klf4, Sox2, Nanog, Lin28, c-Myc, and L-Myc, wherein none of the first, second, and third circular RNAs encode the same reprogramming factor.

In some embodiments, a method of producing an iPSC comprises contacting a somatic cell with four circular RNAs and maintaining the cell under conditions under which a reprogrammed iPSC is obtained. In some embodiments, the first, second, third, and fourth circular RNAs each encode a reprogramming factor selected from Oct3/4, Klf4, Sox2, Nanog, Lin28, c-Myc, and L-Myc, wherein none of the first, second, third, and fourth circular RNAs encode the same reprogramming factor. In some embodiments, the first circular RNA encodes Oct3/4, the second circular RNA encodes Sox2, the third circular RNA encodes c-Myc, and the fourth circular RNA encodes Klf4. In some embodiments, the first circular RNA encodes Oct3/4, the second circular RNA encodes Sox2, the third circular RNA encodes L-Myc, and the fourth circular RNA encodes Klf4.

In some embodiments, a method of producing an iPSC comprises contacting a somatic cell with four circular RNAs and maintaining the cell under conditions under which a reprogrammed iPSC is obtained. In some embodiments, the first, second, third, and fourth circular RNAs each encode a reprogramming factor selected from Oct3/4, Klf4, Sox2, Nanog, and Lin28, wherein none of the first, second, third, fourth, and fifth circular RNAs encode the same reprogramming factor. In some embodiments, the first circular RNA encodes Oct3/4, the second circular RNA encodes Sox2, the third circular RNA encodes Klf4, and the fourth circular RNA encodes Lin28.

In some embodiments, a method of producing an iPSC comprises contacting a somatic cell with five circular RNAs and maintaining the cell under conditions under which a reprogrammed iPSC is obtained. In some embodiments, the first, second, third, fourth, and fifth circular RNAs each encode a reprogramming factor selected from Oct3/4, Klf4, Sox2, Nanog, Lin28, c-Myc, and L-Myc, wherein none of the first, second, third, fourth, and fifth circular RNAs encode the same reprogramming factor.

In some embodiments, the first circular RNA encodes Oct3/4, the second circular RNA encodes Sox2, the third circular RNA encodes Klf4, the fourth circular RNA encodes cMyc, and the fifth circular RNA encodes Lin28.

In some embodiments, a method of producing an iPSC comprises contacting a somatic cell with five circular RNAs and maintaining the cell under conditions under which a reprogrammed iPSC is obtained. In some embodiments, the first, second, third, fourth, and fifth circular RNAs each encode a reprogramming factor selected from Oct3/4, Klf4, Sox2, Nanog, Lin28, wherein none of the first, second, third, fourth, and fifth circular RNAs encode the same reprogramming factor. In some embodiments, the first circular RNA encodes Oct3/4, the second circular RNA encodes Sox2, the third circular RNA encodes Klf4, the fourth circular RNA encodes Lin28, and the fifth circular RNA encodes Nanog. In some embodiments, the first circular RNA encodes Oct3/4, the second circular RNA encodes Sox2, the third circular RNA encodes Klf4, the fourth circular RNA encodes Lin28, and the fifth circular RNA encodes c-Myc. In some embodiments, the first circular RNA encodes Oct3/4, the second circular RNA encodes Sox2, the third circular RNA encodes Klf4, the fourth circular RNA encodes Lin28, and the fifth circular RNA encodes L-Myc.

In some embodiments, a method of producing an iPSC comprises contacting a somatic cell with six circular RNAs and maintaining the cell under conditions under which a reprogrammed iPSC is obtained. In some embodiments, the first, second, third, fourth, fifth, and sixth circular RNAs each encode a reprogramming factor selected from Oct3/4, Klf4, Sox2, Nanog, Lin28, c-Myc, and L-Myc, wherein none of the first, second, third, fourth, fifth, and sixth circular RNAs encode the same reprogramming factor. In some embodiments, the first circular RNA encodes Oct3/4, the second circular RNA encodes Sox2, the third circular RNA encodes Klf4, the fourth circular RNA encodes cMyc, the fifth circular RNA encodes Lin28, and the sixth circular RNA encodes Nanog. In some embodiments, the first circular RNA encodes Oct3/4, the second circular RNA encodes Sox2, the third circular RNA encodes Klf4, the fourth circular RNA encodes L-Myc, the fifth circular RNA encodes Lin28, and the sixth circular RNA encodes Nanog.

In some embodiments, a method of producing an iPSC comprises contacting a somatic cell with six circular RNAs and maintaining the cell under conditions under which a reprogrammed iPSC is obtained. In some embodiments, the first, second, third, fourth, fifth, and sixth circular RNAs each encode a reprogramming factor selected from Oct3/4, Klf4, Sox2, Nanog, Lin28, c-Myc, and L-Myc, wherein none of the first, second, third, fourth, fifth, and sixth circular RNAs encode the same reprogramming factor. In some embodiments, the first circular RNA encodes Oct3/4, the second circular RNA encodes Sox2, the third circular RNA encodes Klf4, the fourth circular RNA encodes cMyc, the fifth circular RNA encodes Lin28 and the sixth circular RNA encodes Nanog. In some embodiments, the first circular RNA encodes Oct3/4, the second circular RNA encodes Sox2, the third circular RNA encodes Klf4, the fourth circular RNA encodes cMyc, the fifth circular RNA encodes Lin28 and the sixth circular RNA encodes Nanog.

In some embodiments, a method of producing an iPSC comprises contacting a somatic cell with seven circular RNAs and maintaining the cell under conditions under which a reprogrammed iPSC is obtained.

In some embodiments, the cell is contacted with multiple circular RNAs, wherein each circular RNA encodes a reprogramming factor selected from the reprogramming factors shown in Table 1, wherein none of the circular RNAs encode the same reprogramming factor.

In some embodiments, the cell is contacted with multiple circular RNAs as shown in Table 3. In Table 3, each row represents a different combination of circular RNAs that may be contacted with a cell, wherein “X” indicates that the circular RNA is contacted with the cell. For example, in combination no. 1, the cell is contacted with a circular RNA encoding Oct3/4 and a circular RNA encoding KLf. In combination no. 104, the cell is contacted with circular RNAs encoding Oct3/4, Klf4, Sox2, and Nanog, Lin28, and L-Myc. In each of the below combos, the cell may optionally additionally be contacted with one or more non-circular RNA nucleic acids encoding one or more reprogramming factors (e.g., one or more plasmids or mRNAs).

TABLE 3
Combinations of Circular RNAs for Generating iPSCs

| Com- | | | | | | | |
|---|---|---|---|---|---|---|---|
| bination | Circ | | | Circ | Circ | Circ | Circ |
| No. | Oct3/4 | circKlf4 | circSox2 | Nanog | Lin28 | C-Myc | L-Myc |
| 1 | X | X | | | | | |
| 2 | X | | X | | | | |
| 3 | X | | | X | | | |
| 4 | X | | | | X | | |
| 5 | X | | | | | X | |
| 6 | X | | | | | | X |
| 7 | | X | X | | | | |
| 8 | | X | | X | | | |
| 9 | | X | | | X | | |
| 10 | | X | | | | X | |
| 11 | | X | | | | | X |
| 12 | | | X | X | | | |
| 13 | | | X | | X | | |
| 14 | | | X | | | X | |
| 15 | | | X | | | | X |
| 16 | | | | X | X | | |
| 17 | | | | X | | X | |
| 18 | | | | X | | | X |
| 19 | | | | | X | X | |
| 20 | | | | | X | | X |
| 21 | | | | | | X | X |
| 22 | X | X | X | | | | |
| 23 | X | X | | X | | | |
| 24 | X | X | | | X | | |
| 25 | X | X | | | | X | |
| 26 | X | X | | | | | X |
| 27 | X | | X | X | | | |
| 28 | X | | X | | X | | |
| 29 | X | | X | | | X | |
| 30 | X | | X | | | | X |
| 31 | X | | | X | X | | |
| 32 | X | | | X | | X | |
| 33 | X | | | X | | | X |
| 34 | X | | | | X | X | |
| 35 | X | | | | X | | X |
| 36 | X | | | | | X | X |
| 37 | | X | X | X | | | |
| 38 | | X | X | | X | | |
| 39 | | X | X | | | X | |
| 40 | | X | X | | | | X |
| 41 | | X | | X | X | | |
| 42 | | X | | X | | X | |
| 43 | | X | | X | | | X |
| 44 | | X | | | X | X | |
| 45 | | X | | | X | | X |
| 46 | | X | | | | X | X |
| 47 | | | X | X | X | | |
| 48 | | | X | X | | X | |
| 49 | | | X | X | | | X |
| 50 | | | X | | X | X | |
| 51 | | | X | | X | | X |
| 52 | | | X | | | X | X |
| 53 | | | | X | X | X | |
| 54 | | | | X | X | | X |
| 55 | | | | X | | X | X |
| 56 | | | | | X | X | X |
| 57 | X | X | X | X | | | |
| 58 | X | X | X | | X | | |
| 59 | X | X | X | | | X | |
| 60 | X | X | X | | | | X |
| 61 | X | X | | X | X | | |
| 62 | X | X | | X | | X | |
| 63 | X | X | | X | | | X |
| 64 | X | X | | | X | X | |
| 65 | X | X | | | X | | X |
| 66 | X | X | | | | X | X |
| 67 | X | | X | X | X | | |
| 68 | X | | X | X | | X | |
| 69 | X | | X | X | | | X |
| 70 | X | | X | | X | X | |
| 71 | X | | X | | X | | X |
| 72 | X | | X | | | X | X |
| 73 | X | | | X | X | X | |
| 74 | X | | | X | X | | X |
| 75 | X | | | X | | X | X |
| 76 | X | | | | X | X | X |
| 77 | | X | X | X | X | | |
| 78 | | X | X | X | | X | |
| 79 | | X | X | X | | | X |
| 80 | | X | | X | X | X | |
| 81 | | X | | X | X | | X |
| 82 | | X | | X | | X | X |
| 83 | | X | | | X | X | X |
| 84 | | | X | X | X | X | |
| 85 | | | X | X | X | | X |
| 86 | | | | X | X | X | X |
| 87 | X | X | X | X | X | | |
| 88 | X | X | X | X | | X | |
| 89 | X | X | X | X | | | X |
| 90 | X | X | X | | X | X | |
| 91 | X | X | X | | X | | X |
| 92 | X | X | X | | | X | X |
| 93 | X | | X | X | X | X | |
| 94 | X | | X | X | X | | X |
| 95 | X | X | | X | X | X | |
| 96 | X | X | | X | X | | X |
| 97 | X | X | | | X | X | X |
| 98 | | X | X | X | X | X | |
| 99 | | X | | X | X | X | X |
| 100 | X | X | X | X | X | X | |
| 101 | X | | X | X | X | X | X |
| 102 | X | X | | X | X | X | X |
| 103 | X | X | X | | X | X | X |
| 104 | X | X | X | X | X | | X |
| 105 | | X | X | X | X | X | X |
| 106 | X | X | X | X | X | X | X |

In some embodiments, a method of producing an IPSC comprises contacting a somatic cell with the circular RNAs of Combination No. 100 in Table 3, above. In some embodiments, a method of producing an IPSC comprises contacting a somatic cell with a combination of circular RNAs that to does not include any circular RNAs expressing C-Myc or L-Myc. In some such embodiments, the combination is selected from a combination listed in Table 3, above, that includes C-Myc and/or L-Myc, but that combination is modified to omit the C-Myc and/or the L-Myc.

In some embodiments, a method of producing an IPSC comprises contacting a somatic cell with a circular RNA encoding Oct4, and additionally contacting the somatic cell with one or more linear RNAs encoding a differentiation factor, circular RNAs encoding a differentiation factor, or viral vectors encoding a differentiation factor. In some embodiments, the level of Oct4 expression is lower compared to a similar method wherein a linear RNA encoding Oct4 is contacted with the cell. In some embodiments, Oct4 expression lasts for a longer period of time, as compared to a similar method wherein a linear RNA encoding Oct4 is contacted with the cell.

In some embodiments, a method of producing an IPSC comprises contacting a somatic cell with one or more circular RNAs encoding a reprogramming factor as described above (e.g., in Table 3), and further comprises contacting the cell with one or more additional circular RNAs. In some embodiments, the one or more additional circular RNAs are selected from circBIRC6, circCORO1C, and circMAN1A2. In some embodiments, the additional circular RNA is circBIRC6. In some embodiments, the additional circular RNA is circCORO1C, and in some embodiments, the additional circular RNA is circMAN1A2.

In some embodiments, a method of producing an iPSC comprises contacting a somatic cell with one or more circular RNAs encoding a reprogramming factor as described above (e.g., in Table 3), and further comprises contacting the cell with the B18R protein, or a circular RNA encoding the B18R protein. In some embodiments, a method of producing an iPSC comprises contacting a somatic cell with one or more circular RNAs encoding a reprogramming factor as described above (e.g., in Table 3), one or more additional circular RNAs selected from circBIRC6, circCORO1C, and circMAN1A2, and the B18R protein, or a circular RNA encoding the B18R protein. The B18R protein, which is encoded by the B18R open reading frame in the Western Reserve (WR) strain of vaccinia virus, is a type I interferon (IFN)-binding protein that is known to inhibit IFN response, and to protect cells from the effects of interferon. An exemplary B18R sequence is provided as SEQ ID NO: 16. In some embodiments, the B18R protein has a sequence that is at least 90%, or at least 95% identical to SEQ ID NO: 16.

In some embodiments, a method of producing an iPSC comprises contacting a somatic cell with one or more circular RNAs encoding a reprogramming factor as described above (e.g., in Table 3), and further comprises contacting the cell with one or more additional reprogramming factors. The additional reprogramming factor may be, for example, a non-coding RNA (e.g., LINcRNA-ROR, miR302 (miR302d, miR302a, miR302c or miR302b), miR367, miR766, miR200c, miR369, miR372, Let7, miR19a/b), vitamin C, valproic acid, CHIR99021, Parnate, SB431542, PD0325901, BIX-01294, Lithium Maxadilan, 8-Br-cAMP, A-83-01, Tiazovivin, Y-27632, EPZ004777, or DAPT.

In some embodiments, a method for reprogramming a cell may comprise contacting the cell with: (i) at least one circular RNA encoding a reprogramming factor, (ii) at least one circular RNA that does not encode any protein or miRNA, (iii) at least one circular or linear RNA encoding a miRNA, and/or (iv) at least one circular or linear RNA encoding a viral protein, in any combination. The at least one reprogramming factor may be, for example, any one of the reprogramming factors listed in Table 1. The at least one circular RNA that does not encode any protein or miRNA may be, for example, circBIRC6 (SEQ ID NO: 13), circCORO1C (SEQ ID NO: 14), and/or circMAN1A2 (SEQ ID NO: 15). The miRNA may be, for example, a miRNA of the miRNA302 family (e.g., miR302d, miR302a, miR302c and miR302b) or miR367. The viral protein may be, for example, B18R, E3 or K3.

In some embodiments, a method for reprogramming a cell may comprise treating the cell to suppress or prevent an innate immune response. For example, a method for reprogramming a cell may comprise contacting the cell with one or more viral proteins that inhibit the innate immune response, or circular RNA(s) encoding the viral protein(s). The viral proteins may be, for example, inhibitors of RIG-1 (retinoic acid-inducible gene I) or PKR (protein kinase R) pathways. Exemplary viral proteins suitable for use in the methods described herein include, but are not limited to, B18R, E3, or K3 from vaccinia virus. Additional viral proteins are listed below in Table 4.

TABLE 4
Viral proteins for suppressing an innate immune response

| Protein | Virus |
|---|---|
| Gamma 34.5 | Herpes simplex virus (HSV) |
| VP35 | Ebola virus |
| Influenza NS1 | Influenza virus |
| pTRS1/pIRS1 | Human cytomegalovirus (CMV) |
| m142/m143 | Murine CMV |
| NSs | Rift Valley fever virus (RVFV) |
| E3L | Vaccinia virus |
| MC159L | Poxvirus |
| NSP3 | Rotavirus group C |
| NSP5 | Rotavirus group 1 |
| Us11 | Herpes simplex virus |
| SM | Epstein-Barr virus |
| OVIFNR | Parapoxvirus |
| Crm1 | Poxvirus |
| L(pro) | Foot-and-mouth disease virus |
| Us11 | Herpex simplex virus |
| E6 | Papilloma virus |
| Large T antigen | SV-40 |
| LANA2 | Herpes Virus |
| BILF1 | Epstein-Barr virus |
| NS5A | Hepatitis C virus |
| P58 | Influenza virus |
| SM | Epstein-Barr virus |
| VIRF-2 | Human herpes virus-8 |
| PK2 | Baculovirus |
| TAT | HIV-1 |
| K3L | Vaccinia virus, Iridoviridae |
| S-HDAg | Hepatitis D virus |
| E2 | Hepatitis C virus |
| C8L | Swinepox virus |

Another way to suppress or prevent an innate immune response is to treat the cell with a miRNA (or a circular RNA encoding the miRNA) that targets RIG-1 (retinoic acid-inducible gene I) or PKR (protein kinase R). The miRNA may be, for example, miR146a, miR485, miR182, nc886, miR-155, miR526a, or miR132. In some embodiments, a method for reprogramming a cell may comprise treating the cell with an miRNA or a circular RNA encoding the same, wherein the miRNA targets RIG-1 or PKR.

Illustrative combinations of RNAs for use in a method of reprogramming a cell are shown below in Table 5. In Table 5, each row represents a different combination that may be contacted with a cell, wherein “X” indicates that the RNA is contacted with the cell. For example, in combination no. 1, the cell is contacted with a circular RNA encoding a reprogramming factor. In combination no. 15, the cell is contacted with a circular RNA encoding a reprogramming factor, a circular RNA that does not encode any protein or miRNA, a circular or linear RNA encoding a miRNA, and a circular or linear RNA encoding a viral protein.

TABLE 5
Combinations of RNAs for use in a method of reprogramming a cell

| | | Circular RNA that | | |
|---|---|---|---|---|
| | | does not encode | Circular or linear | |
| | Circular RNA | any protein or | RNA encoding a | |
| | encoding a | miRNA (e.g., | miRNA (e.g., | Circular or linear |
| | reprogramming | circBIRC6, | miR302d, miR302a, | RNA encoding a |
| Combination | factor (See, | circCORO1c, | miR302c, miR302b, | viral protein (e.g., |
| No. | e.g., Table 1) | circMAN1A2) | or miR367) | B18R, E3, K3) |
| 1 | X | | | |
| 2 | | X | | |
| 3 | | | X | |
| 4 | | | | X |
| 5 | X | X | | |
| 6 | X | | X | |
| 7 | X | | | X |
| 8 | | X | X | |
| 9 | | X | | X |
| 10 | | | X | X |
| 11 | X | X | X | |
| 12 | X | X | | X |
| 13 | X | | X | X |
| 14 | | X | X | X |
| 15 | X | X | X | X |

The contacting may be performed by any of the methods described above, such as by transfection, electroporation, and/or the use of circRNA-LNP complexes. In some embodiments, the contacting comprises incubating the cell with one or more circular RNAs, such as circular RNAs encoding reprogramming factors.

In some embodiments, the circular RNA is contacted with the cells once. In some embodiments the circular RNA is contacted with the cells more than once, e.g., 2, 3, 4, 5, 6, 7, 8, 9, or 10 times. In some embodiments, the contacting is performed at effective intervals. The effective intervals may be, for example, once per day, once every other day, once every three days, once per week, once every two weeks, or once per month. In some embodiments, the circular RNA is contacted with the cells for the duration of the reprogramming process, such that the contact is continuous throughout the reprogramming process.

As explained above, the methods for producing iPSCs may comprise maintaining the cell under conditions under which a reprogrammed iPSC is obtained. Such conditions are known to those of skill in the art, and may vary by cell type. As one example, somatic cells may first be placed into a flask with the appropriate medium so that they are about 75% to about 90% confluent on the day that they are contacted with the circRNAs (Day 0). The cells may then be contacted with the circRNAs (e.g., by transfection). The transfected cells may be plated onto culture disks and incubated overnight. For the next 10-14 days, the media may be changed as required. In some embodiments, media may be supplemented with one or more additional agents to enhance cellular reprogramming. The cells may be monitored for the emergence of iPSC colonies, and iPSC colonies are picked and transferred into separate dishes for expansion.

To confirm the pluripotency of the iPSCs, isolated clones can be tested for the expression of one or more stem cell markers. Stem cell markers can be selected from, for example, Oct4, Lin28, SOX2, SSEA4, SSEA3, TRA-1-81, TRA-1-60, CD9, Nanog, Fbxl5, Ecatl, Esgl, Eras, Gdf3, Fgf4, Cripto, Daxl, Zpf296, Slc2a3, Rexl, Utfl, and Nat1. Methods for detecting the expression of such markers can include, for example, RT-PCR and immunological methods that detect the presence of the encoded polypeptides.

In some embodiments, the pluripotency of the cell is confirmed by measuring the ability of the cells to differentiate to cells of each of the three germ layers. In some embodiments, teratoma formation in immunocompromised rodents can be used to evaluate the pluripotent character of the isolated clones.

In some embodiments, circRNA reprogramming requires less frequent and/or a smaller number of transfections (as compared to linear RNA-based approaches) to achieve iPSC reprogramming. For example, circRNA reprogramming may require about 10%, about 20%, about 30%, about 40%, about 50%, about 60%, about 70%, about 80%, about 90%, or about 100% fewer transfections, as compared to linear RNA-based approaches, to achieve reprogramming.

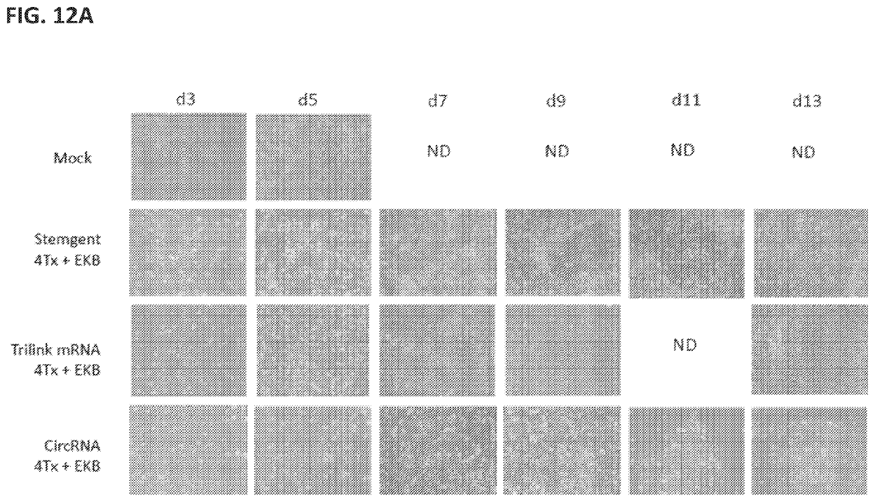

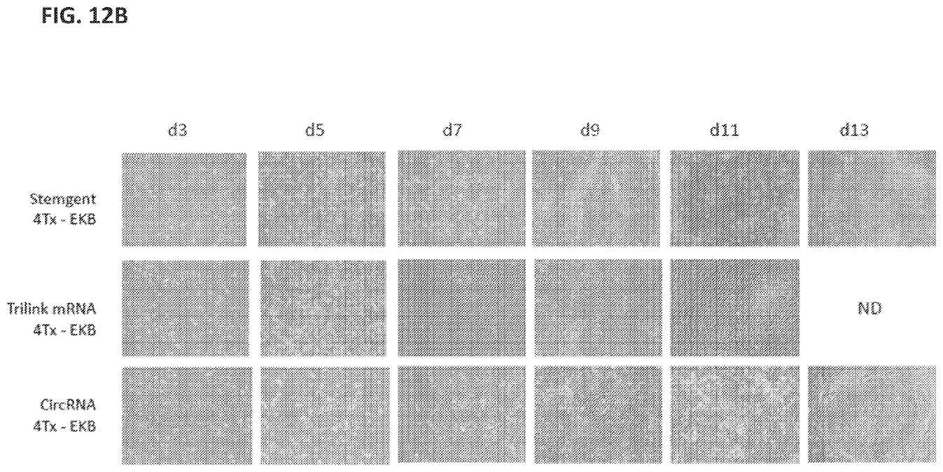

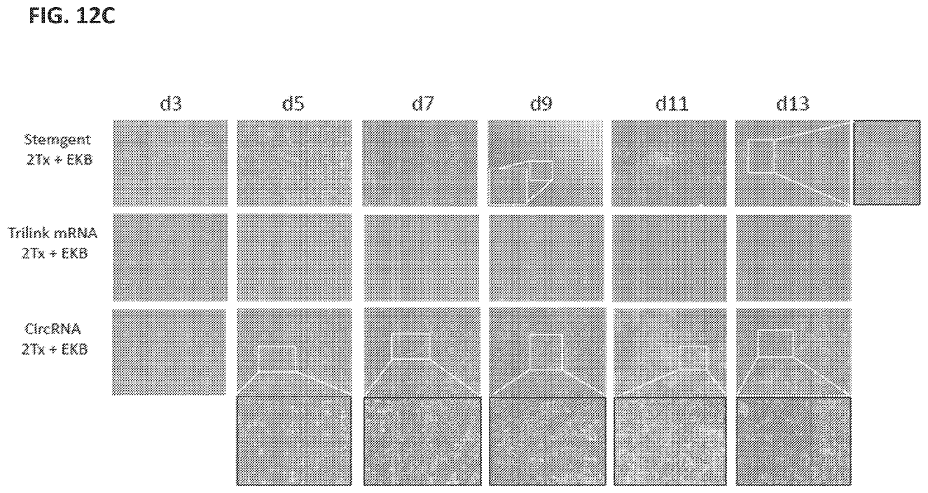

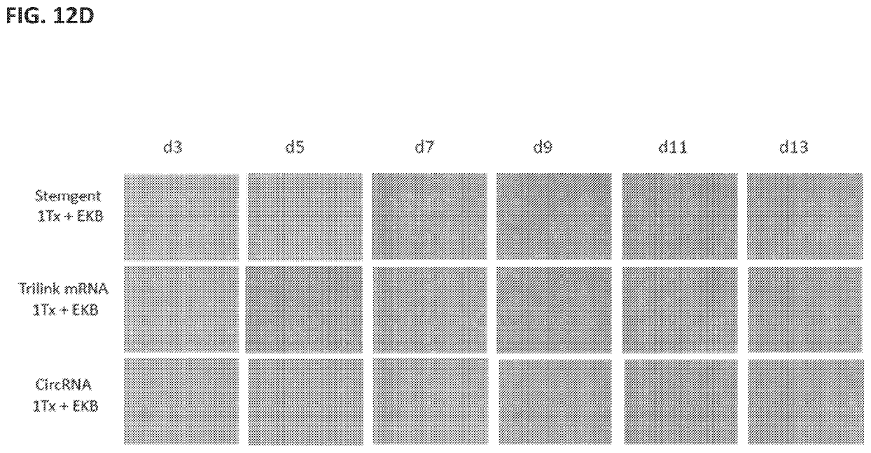

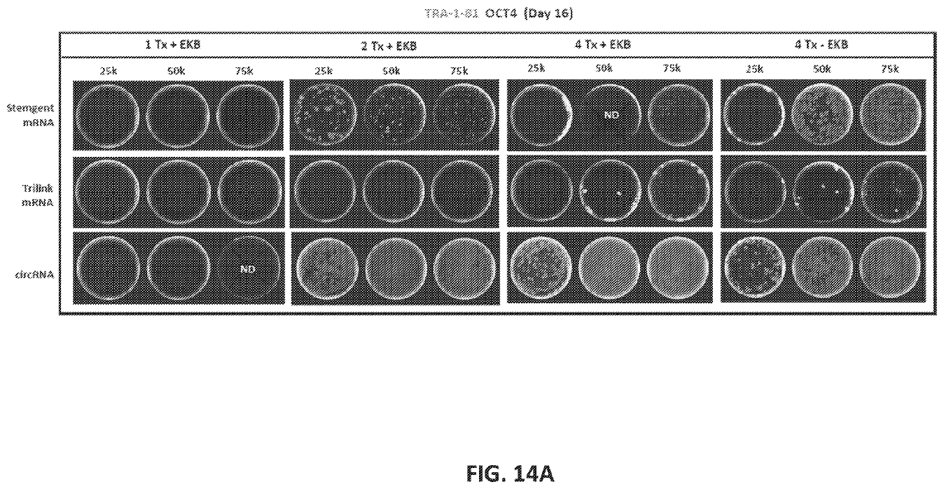

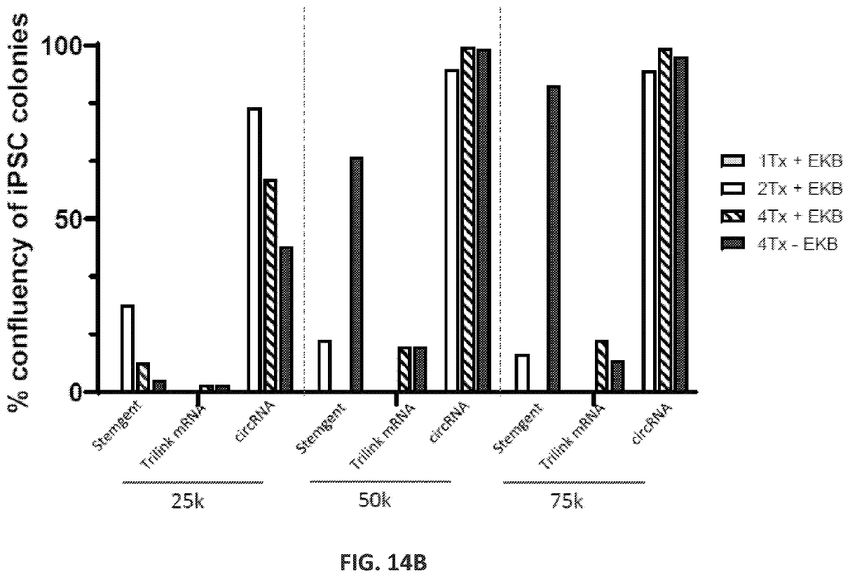

In some embodiments, circRNA reprogramming results in enhanced reprogramming efficiency compared to linear RNA-based approaches. “Reprogramming efficiency” refers to a quantitative or qualitative measure of iPSC generation from a starting population of cells. Read-outs of reprogramming efficiency include quantitation of the number of iPSC colonies present at a particular timepoint during a reprogramming protocol (as an assessment of the rate of colony formation) or at the completion of a reprogramming protocol (as an assessment of the total number of iPSC colonies generated during a particular protocol). See e.g., Example 6 and FIG. 12. iPSC colonies can be identified quantitatively (such as by staining with cell surface markers of pluripotency and counting the number of stained cells—See FIG. 14) or qualitatively by assessment of morphological characteristics (e.g., tightly-packed cells with each cell in the colony having a more or less uniform shape and diameter, colonies comprising a clearly-defined border, and cells within iPSC colonies comprising a high nuclear to cytoplasmic ratio and prominent nucleoli). Reprogramming efficiency may also include an assessment of the relative maturity of iPSCs colonies between various reprogramming protocols. Maturation of iPSC colonies can be determined by the morphological characteristics noted above.

An increase in reprogramming efficiency refers to an increase in one or more read-outs of reprogramming efficiency when two or more reprogramming protocols are compared. For example, and as detailed in the Examples, reprogramming with circRNA-encoded reprogramming factors results in an increase in reprogramming efficiency compare to reprogramming with linear RNA-encoded reprogramming factors.

In some embodiments, increased reprogramming efficiency comprises an increase in the total number of iPSC colonies present at the end of a first reprogramming protocol compared to the total number of iPSC colonies present at the end of a second and/or third reprogramming protocol. In some embodiments, increased reprogramming efficiency comprises an increase in the total number of iPSC colonies present at a particular timepoint a first reprogramming protocol compared to the total number of iPSC colonies present at the same timepoint in a second and/or third reprogramming protocol (i.e., an increase in the rate of iPSC colony formation).

In some embodiments, the cell is a prokaryotic cell. In some embodiments, the cell is a eukaryotic cell. In some embodiments, the cell is a mammalian cell (e.g., a murine, bovine, simian, porcine, equine, ovine, or human cell). In some embodiments, the cell is a human cell. In some embodiments, the cell is a yeast, fungi, or plant cell.

In some embodiments, the cell is a somatic cell. In some embodiments, the cell is a fibroblast, a peripheral blood-derived cell, an endothelial progenitor cell, a cord-blood derived cell, a hepatocyte, a keratinocyte, a melanocyte, an adipose-tissue derived cell, or a urine-derived cell (e.g., a renal epithelial progenitor cell). In some embodiments, the cell is an epithelial cell, an endothelial cell, a neuronal cell, an adipose cell, a cardiac cell, a skeletal muscle cell, an immune cell, a hepatic cell, a splenic cell, a lung cell, a circulating blood cell, a gastrointestinal cell, a renal cell, a bone marrow cell, a progenitor cell, or a pancreatic cell. In some embodiments, the cell is isolated from a somatic tissue including, but not limited to brain, liver, lung, gut, stomach, intestine, fat, muscle, uterus, skin, spleen, endocrine organ, bone, etc. In some embodiments, the cell is an amniotic fluid cell, an adipose stem cell, a dental pulp cell, or a pancreatic islet beta cell.

In some embodiments, the cell is an adherent cell. In some embodiments, the cell is a non-adherent cell (i.e., a suspension cell such as a CD34+ cell).

### Methods for Transdifferentiating Cells

Additionally provided herein are methods for transdifferentiating cells using circular RNAs. In some embodiments, a method of directly converting a cell from a first cell type to a second cell type comprises contacting the cell with the recombinant circular RNA or composition as described herein, and maintaining the cell under conditions under which the cell is converted to the second cell type. In some embodiments, the cell does not enter an intermediate pluripotent state. In some embodiments, the cell is converted directly from the first cell type to the second cell type, without becoming a progenitor cell.

In some embodiments, the circular RNA encodes one or more reprogramming factors that are capable of transdifferentiating cells from a first cell type to a second cell type. In some embodiments, the circular RNA encodes MyoD, C/EBPα, C/EBPβ, Pdx1, Ngn3, Mafa, Pdx1, Hnf4a, Foxa1, Foxa2, Foxa3, Ascl1 (also known as Mash1), Brn2, Myt1l, miR-124, Brn2, Myt1l, Ascl1, Nurr1, Lmx1a, Ascl1, Brn2, Myt1l, Lmx1a, FoxA2, Oct4, Sox2, Klf4 and c-Myc, Tbx5, Mef2c, Gata-4, and/or Mesp1. In some embodiments, the circular RNA encodes one or more reprogramming factors listed in Table 1.

In some embodiments, the first cell type is an iPSC. In some embodiments, the first cell type is a differentiated fibroblast.

In some embodiments, the second cell type is a muscle cell, a neuron, a cardiomyocyte, a hepatocyte, an islet, a keratinocyte, a T-cell, or a NK-cell. In some embodiments, the second cell type is a muscle cell, a neuron, a cardiomyocyte, a hepatocyte, an islet cell, a keratinocyte, a T-cell, or a NK-cell.

In some embodiments, a method of directly converting a cell from a first cell type to a second cell type comprises contacting the cell with multiple circular RNAs, wherein each circular RNA encodes a transdifferentiation factor according to one of the combinations listed in Table 6.

In some embodiments, a method of directly converting a cell from a first cell type to a second cell type comprises contacting the cell with multiple circular RNAs wherein each circular RNA encodes a transdifferentiation factor listed in Table 6. In some embodiments, the cell is contacted with at least 2, at least 3, at least 4, at least 5, at least 6, at least 7, at least 8, at least 9, at least 10, or more circular RNAs.

In some embodiments, a method of directly converting a cell from a first cell type to a second cell type comprises contacting the cell with two circular RNAs, and maintaining the cell under conditions under which the cell is converted to the second cell type. In some embodiments, the first and second circular RNAs each encode a transdifferentiation factor listed in Table 6, wherein the first and second circular RNAs do not encode the same transdifferentiation factor.

In some embodiments, a method of directly converting a cell from a first cell type to a second cell type comprises contacting the cell with three circular RNAs, and maintaining the cell under conditions under which the cell is converted to the second cell type. In some embodiments, the first, second, and third circular RNAs each encode a transdifferentiation factor listed in Table 6, wherein the first, second, and third circular RNAs do not encode the same transdifferentiation factor.

In some embodiments, a method of directly converting a cell from a first cell type to a second cell type comprises contacting the cell with four circular RNAs, and maintaining the cell under conditions under which the cell is converted to the second cell type. In some embodiments, the first, second, third, and fourth circular RNAs each encode a transdifferentiation factor listed in Table 6, wherein the first, second, third, and fourth circular RNAs do not encode the same transdifferentiation factor.

In some embodiments, a method of directly converting a cell from a first cell type to a second cell type comprises contacting the cell with five circular RNAs, and maintaining the cell under conditions under which the cell is converted to the second cell type. In some embodiments, the first, second, third, fourth, and fifth circular RNAs each encode a transdifferentiation factor listed in Table 6, wherein the first, second, third, fourth and fifth circular RNAs do not encode the same transdifferentiation factor.

In some embodiments, a method of directly converting a cell from a first cell type to a second cell type comprises contacting the cell with six circular RNAs, and maintaining the cell under conditions under which the cell is converted to the second cell type. In some embodiments, the first, second, third, fourth, fifth, and sixth circular RNAs each encode a transdifferentiation factor listed in Table 6, wherein the first, second, third, fourth, fifth and sixth circular RNAs do not encode the same transdifferentiation factor.

In some embodiments, a method of directly converting a cell from a first cell type to a second cell type comprises contacting the cell with seven circular RNAs, and maintaining the cell under conditions under which the cell is converted to the second cell type. In some embodiments, the first, second, third, fourth, fifth, and sixth circular RNAs each encode a transdifferentiation factor listed in Table 6, wherein the first, second, third, fourth, fifth, and sixth circular RNAs do not encode the same transdifferentiation factor.

In some embodiments, a method of directly converting a cell from a first cell type to a second cell type comprises contacting the cell with multiple circular RNAs, and maintaining the cell under conditions under which the cell is converted to the second cell type. In some embodiments, each of the circular RNAs each encode a transdifferentiation factor listed in Table 6, wherein none of the circular RNAs encode the same transdifferentiation factor.

In some embodiments, a cell is contacted with a circular RNA encoding one or more reprogramming factors listed in Table 6. In some embodiments, a method of directly converting a cell from a first cell type as shown in Table 6 to a second cell type as shown in Table 6 comprises contacting the cell with the recombinant circular RNA encoding one or more reprogramming factors listed in Table 6, and maintaining the cell under conditions under which the cell is converted to the second cell type. The first cell type may be, for example, any of the cell types listed in Table 6. The second cell type may be, for example, any of the cell types listed in Table 6.

In some embodiments, the present disclosure provides a composition comprising one or more circular RNAs, wherein each circular RNA encodes one or more of the transdifferentiation factors listed in Table 6. In some embodiments, the present disclosure provides a composition comprising a plurality of circular RNAs, each circular RNA encoding at least one transdifferentiation factor listed in Table 6.

In some embodiments, a method for transdifferentiating a cell comprises contacting a cell with one or more circular RNAs, wherein each of the circular RNAs encodes a transdifferentiation factor listed in Table 6. In some embodiments, a method for transdifferentiating a cell comprises contacting a cell with one or more circular RNAs, wherein each of the circular RNAs encodes a transdifferentiation factor listed in Table 6, and wherein the cell is any one of the “first cell type” listed in Table 6.

In some embodiments, a method for transdifferentiating a cell comprises contacting the first cell type listed Column A of any Combination No. shown in Table 6 with the corresponding transdifferentiation factor(s) shown in Column B of that same transdifferentiation combination to produce the second cell type shown in Column C of that same Combination No., wherein at least one transdifferentiation factor shown in Column B is encoded by a circular RNA. In some embodiments, all of the transdifferentiation factor(s) shown in Column B for a given transdifferentiation combination are encoded by one or more circularized RNA(s). In some embodiments, a first cell type is transdifferentiated to a second cell type using the transdifferentiation factors listed in Column B for any one of Combination Nos. 1-151. In some embodiments, the first cell type is any one of the cell types listed Column A for any one of Combination Nos. 1-151. In some embodiments, the second cell type is any one of the second cell types listed in Column C for any one of Combination Nos. 1-151.

TABLE 6
Exemplary transdifferentiation factors for converting
a cell from a first cell type to a second cell type

| Combination | Column A | Column B | Column C |
|---|---|---|---|
| No | First cell type | Transdifferentiation factor(s) | Second cell type |
| 1 | Fibroblast | MyoD | Myocyte |
| 2. | B-cell | C/EBPα, C/EBPβ | Macrophage |
| 3. | Pancreatic duct cell | Pdx1 | Beta cell |
| 4. | Pancreatic exocrine | Ngn3, Mafa, Pdx1 | |
| | cell | | |
| 5. | Hepatocyte | Exendin-4, Pdx1 | |
| 6. | Fibroblast | Hnf4α, Foxa1 or Foxa2 or | Hepatocyte |
| | | Foxa3 | |
| 7. | Fibroblast | Ascl1 (also known as Mash1), | Neuron |
| | | Brn2, Myt1l, miR-124, Brn2, | |
| | | Myt1l | |
| 8 | Astrocyte | Pax6, neurogenin 2, Ascl1 | |
| 9. | Fibroblast | Ascl1, Nurr1, Lmx1a, Ascl1, | Dopaminergic neuron |
| | | Brn2, Myt1l, Lmx1a, FoxA2 | |
| 10. | Fibroblast | Oct4, Sox2, Klf4 and c-Myc, | Cardiomyocyte |
| | | Tbx5, Mef2c, Gata-4, Mesp1 | |
| 11. | Human Adult | Brn2, Mty1l, miRNA-124 | Neurons |
| | Dermal Fibroblast | | |
| 12. | Human Adult | Ascl1, Brn2, Myt1l, Ngn2 | |
| | Peripheral | | |
| | Blood Mononuclear | | |
| | Cells | | |
| 13. | Human Striatum | Ascl1, Brn2, Myt1l | |
| | Astrocytes | | |
| 14. | Murine Embryonic | Ascl1, Brn2, Myt1l | |
| | and | | |
| | Postnatal | | |
| | Fibroblasts | | |
| 15. | Human Neonatal | Foxa2, Hnf4α, C/EBPβ, c-Myc | Hepatocytes |
| | Fibroblasts | | |
| 16. | Human Embryonic | Hnf1α, Hnf4α, Foxa3 | |
| | Fibroblasts | | |
| 17. | Human Adult | ETV2 | Endothelial Cells |
| | Fibroblasts | | |
| 18. | Murine Amniotic | Sox17 | |
| | Cells | | |
| 19. | Human Newborn | Oct4, Sox2, KLF4, c-Myc | |
| | Dermal | bFGF, βME | |
| | and Lung | | |
| | Fibroblasts | | |
| 20. | Murine Embryonic | Myod1 | |
| | Fibroblasts | | |
| 21. | Human Dermal | Myod1 | |
| | Fibroblasts | SB431542, Chir99021, EGF, | |
| | | IGF1 | |
| 22. | Human Dermal | Cartilage-derived | Chondrocytes |
| | Fibroblasts | morphogenetic protein 1 | |
| 23. | Mouse Dermal | c-Myc, KLF4, Sox9 | |
| | Fibroblast | | |
| 24. | Murine Adult | Pdx1, Ngn3, Mafa | Pancreatic β-Cells |
| | Pancreatic | | |
| | Exocrine Cells | | |
| 25. | Human Pancreatic | MAPK, STAT3 | |
| | Exocrine Cells | | |
| 26. | Murine Cardiac | Gata4, Mef2c, Tbx5 | Cardiomyocytes |
| | Fibroblasts | | |
| 27. | Murine Cardiac | miRNA-1, miRNA-133, | |
| | Fibroblasts | miRNA-208, miRNA-499 | |
| 28. | Murine Myoblasts | Myod1 | Adipocytes |
| 29. | Murine Adipose | Runx2 | |
| | Tissue-Derived | | |
| | Stem Cells | | |
| 30. | Murine | Runx2, MKP-1 | |
| | Preadipocytes | | |
| 31. | Astrocytes | Pax6, Mash1, or Ngn2 | Glutamatergic |
| | | | neurons |
| 32. | Embryonic | Brn2, Ascl1, and Myt1l | Neuronal cells |
| | fibroblasts and | | |
| | Hepatocytes | | |
| 33. | Astrocytes | Dlx2; Dlx2 and Ascl1 | GABAergic neurons |
| 34. | Embryonic | Ascl1, Lmx1a, and Nurr1 | Dopaminergic neurons |
| | fibroblasts and | | |
| | Adult skin | | |
| | fibroblasts | | |
| 35. | Fetal fibroblasts | BRN2, ASCL1, MYT1L, and | Neuronal cells |
| | and Postnatal | NEUROD1 | |
| | foreskin fibroblasts | | |
| 36. | Embryonic | ASCL1, BRN2, MYT1L, | Neuronal cells |
| | fibroblasts and | LMX1A, and FOXA2 | |
| | Postnatal | | |
| | fibroblasts | | |
| 37. | Embryonic | Brn4/Pou3f4, Sox2, Klf4, c- | Neural stem cells |
| | fibroblasts | Myc, and E47/Tcf3 | |
| 38. | Embryonic | Sox2 | Neural stem cells |
| | fibroblasts and | | |
| | Fetal foreskin | | |
| | fibroblasts | | |
| 39. | Sertoli cells | Pax6, Ngn2, Hes1, Id1, Ascl1, | Neural stem cells |
| | | Brn2, c-Myc, and Klf4 | |
| 40. | Fibroblasts (IMR90 | MASH1, NGN2, SOX2, | Dopaminergic neurons |
| | cells) | NURR1, and PITX3 + A | |
| | | dominant-negative P53 | |
| 41. | Non-sensory | Ascl1; Ascl1 and Neurod | Neuronal cells |
| | cochlear epithelial | | |
| | cells | | |
| 42. | Astrocytes | Brn4 | Neuronal cells |
| 43. | Skin fibroblasts | Brn2, Sox2, and Foxa2 | Dopaminergic |
| | | | precursors |
| 44. | Adult skin | NEUROG2, SOX11, ISL1, and | Motor neurons |
| | fibroblasts | LHX3 | |
| 45. | Fibroblasts (3T6 | Ascl1, Brn4, and Tcf3 | Neuronal cells |
| | cells) | | |
| 46. | Umbilical cord | SOX2 and HMGA2 | Neural stem cells |
| | blood cells | | |
| 47. | Fibroblasts (3T6 | Ascl1, Brn2, and Foxa1 | Neuronal cells |
| | cells) | | |
| 48. | Resident glial cells | Ascl1, Lmx1a, and Nurr1 | Neuronal cells |
| 49. | Fibroblast-like cells | ASCL1 and PAX6 | Neuronal cells |
| | from retinal tissues | | |
| 50. | Pluripotent stem | Brn2, Ascl1, Myt1l and Neurod | Neuronal cells |
| | cell-derived | | |
| | cardiomyocytes | | |
| 51. | Fibroblasts | ASCL1, ISL1, NEUROD1, | Motor neurons |
| | | BRN2, HB9, LHX3, MYT1L, | |
| | | and NGN2 | |
| 52. | Fibroblasts | SOX2, GATA3, and | Neurocytes |
| | | NEUROD1 | |
| 53. | Dermal fibroblasts | SOX2 and PAX6 | Neural precursor cells |
| 54. | Embryonic | Ptf1a | Neural stem cells |
| | fibroblasts and | | |
| | Newborn foreskin | | |
| | fibroblasts | | |
| 55. | Adult fibroblasts | SOX2; SOX2 and PAX6; | Neural precursor cells |
| | | SOX2 and LMX1A; SOX2, | |
| | | LMX1A, and FOXA2 | |
| 56. | Spiral ganglion | Ascl1 and Neurod | Neuronal cells |
| | non-neuronal cells | | |
| 57. | Umbilical cord | SOX2, ASCL1, and | Neuronal cells |
| | mesenchymal stem | NEUROG2 | |
| | cells | | |
| 58. | Pericytes | ASCL1 and SOX2 | Neuronal cells |
| 59. | Cord blood | FOXM1, SOX2, MYC, SALL4, | Neuronal cells |
| | CD133(+) cells | and STAT6 | |
| 60. | Hepatocytes | Suz12, Ezh2, Meis1, Sry, | Neuronal cells |
| | | Smarca4, Esr1, Pparg, and | |
| | | Stat3 | |
| 61. | Peripheral CD34(+) | AR, SOX2, SMAD3, MYC, | Neural stem cells |
| | cells | JUN, WT1, TAL1, SPI1, and | |
| | | RUNX1 | |
| 62 | Urine epithelial-like | POU3F2, SOX2, BACH1, AR, | Neural stem cells |
| | cells | PBX1, and NANOG | |
| 63. | Muller glia cells | Bmi1, Spi1, Lmo2, and Cebpd | Neural stem cells |
| 64. | Astrocytes and | Ascl1, Phox2b, Ap-2a, Gata3, | Noradrenergic |
| | Foreskin fibroblasts | Hand2, Nurr1, and Phox2a | neurons |
| 65. | Bone marrow- | MSI1, NGN2, and MBD2 | Neural precursor cells |
| | derived cells, | | |
| | Fibroblasts, and | | |
| | Keratino-cytes | | |
| 66. | Microglial cells | Neurod1 | Neuronal cells |
| 67. | Cardiac fibroblasts | Gata4, Mef2c, and Tbx5 | Cardiomyocytes |
| 68. | Cardiac fibroblasts | Gata4, Mef2c, Tbx5, and | Cardiomyocytes |
| | | Hand2 | |
| 69. | Cardiac fibroblasts | Mef2c and Tbx5 + Myocd or | Cardiomyocytes |
| | and Embryonic | Gata4 | |
| | fibroblasts | | |
| 70. | Cardiac fibroblasts | GATA4, MEF2C, TBX5, | Cardiomyocytes |
| | | MESP1, and MYOCD | |
| 71. | Embryonic stem | GATA4, MEF2C, TBX5, | Cardiomyocytes |
| | cells-derived | ESRRG, MESP1, ZFPM2, and | |
| | fibroblasts | MYOCD | |
| 72. | Adult fibroblasts | Gata4, Hand2, Mef2c, Tbx5, | Cardiomyocytes |
| | | and Znf281 | |
| 73. | Embryonic | Hnf4a and Foxa1, Foxa2, or | Hepatocytes |
| | fibroblasts and | Foxa3 | |
| | Adult dermal | | |
| | fibroblasts | | |
| 74. | Caudal fibroblasts | Gata4, Hnf1a, and Foxa3 + | Hepatocytes |
| | | p19^{Arf} knockdown | |
| 75. | Fetal and adult | FOXA3, HNF1A, and HNF4A + | Hepatocytes |
| | fibroblasts and | SV40 large T antigen | |
| | Adipose tissue- | | |
| | derived | | |
| | mesenchymal stem | | |
| | cells | | |
| 76. | Fibroblasts (BJ and | HNF1A and Any two of the | Hepatocytes |
| | MRC-5 cells) | three factors: FOXA1, FOXA3, | |
| | Hepatocytes cells) | and HNF4A | |
| 77. | Liver cells in mouse | Foxa3, Gata4, Hnf1a, and | Hepatocytes |
| | models of chronic | Hnf4a | |
| | liver disease | | |
| 78. | Fetal lung | ATF5, PROX1, FOXA2, | Hepatocytes |
| | fibroblasts | FOXA3, and HNF4A | |
| 79. | Fibroblasts | OCT4, FOXA2, HNF1A, and | Hepatocytes |
| | | GATA3 | |
| 80. | Embryonic | Foxa3, Hnf1a, and Gata4 | Hepatocytes |
| | fibroblasts | | |
| 81. | Embryonic | Hnf4a, Foxa3, Klf4, and c-Myc | Hepatocytes |
| | fibroblasts | | |
| 82. | Liver cells in vivo | Pdx1 | β cells |
| 83. | Pancreatic exocrine | Ngn3, Pdx1, and Mafa | β cells |
| | cells in vivo | | |
| 84. | Hepatocytes | Ngn3 | Islet cells |
| 85. | Liver cells | PDX1, PAX4, and MAFA | β cells |
| 86. | Cultured adult | Pdx1, Ngn3, and Mafa | β cells |
| | pancreatic duct | | |
| | cells | | |
| 87. | Gallbladder cells | Pdx1, Ngn, Mafa, and Pax6 | β cells |
| 88. | T precursor cells | Cebpa or Cebpb | Macrophages |
| 89. | T precursor cells | Pu.1 | Dendritic cells |
| 90. | B cells | Pax5 knockout | T cells |
| 91. | Fibroblasts (3T3 | Pu.1 and Cebpa or Cebpb | Macrophage-like cells |
| | cells), Embryonic | | |
| | fibroblasts, and | | |
| | Adult skin | | |
| | fibroblasts | | |
| 92. | 3 cells | Gata1, Scl, and Cebpa | Erythroid cells |
| 93. | Fibroblasts (3T3 | Nfe2, Mafg, and Mafk | Megakaryocyte |
| | cells) and Adult | | |
| | dermal fibroblasts | | |
| 94. | Skin fibroblasts | Spl1, Cebpa, Mnda, and Irf8 | Monocytes |
| 95. | Embryonic | Erg, Gata2, Lmo2, Runx1c, | Hematopoietic |
| | fibroblasts and | and Scl | progenitor cells |
| | Adult ear skin | | |
| | fibroblasts | | |
| 96. | Fibroblasts | Pu.1, Irf8, and Batf3 | Antigen-presenting |
| | | | dendritic cells |
| 97. | Neonatal foreskin | c-MYC, KLF4, and SOX9 | Chondrogenic cells |
| | fibroblasts | | |
| 98. | Dermal fibroblasts | OCT3/4 and OCT6 or OCT9 + | Osteoblasts |
| | | L-MYC, c-MYC, or N-MYC | |
| 99. | Fibroblasts | RUNX2, OCT4, OSTERIX, and | Osteoblasts |
| | | L-MYC | |
| 100. | Gingival fibroblasts | OCT4, OSTERIX, and L-MYC | Osteoblasts |
| | and Adult dermal | | |
| | fibroblasts | | |
| 101. | Embryonic | c-Myc, Oct4, and hLMP3 | Osteoblasts |
| | fibroblasts | | |
| 102. | Fibroblasts | Myod | Myoblasts |
| | (C3H10T1/2 cells) | | |
| 103. | Dermal fibroblasts | MYOD1 and MYCL | Myoblasts |
| 104. | Embryonic | Mef2b and Pitx1 + Pax3 or | Skeletal muscle |
| | fibroblasts | Pax7 | progenitor cells |
| 105. | Adult fibroblasts | Pax7, Mef2b, and Myod | Skeletal muscle |
| | | | progenitor cells |
| 106. | Embryonic | Prdm16 and Cebpb | Brown fat cells |
| | fibroblasts and | | |
| | Newborn foreskin | | |
| | fibroblasts | | |
| 107. | Embryonic | Nr5a1, Wt1, Dmrt1, Gata4, | Sertoli cells |
| | fibroblasts | and Sox9 | |
| 108. | Iris-derived cells | CRX, RAX, and | Photoreceptor cells |
| | | NEUROD | |
| 109 | Embryonic | Mitf, Sox10, and Pax3 | Melanocytes |
| | fibroblasts and | | |
| | Adult tail-tip dermal | | |
| | fibroblasts | | |
| 110. | Adipose tissue- | SOX18 | Endothelial cells |
| | derived stromal | | |
| | cells | | |
| 111. | Dermal fibroblasts | CRX, RAX, OTX2, and | Photoreceptor cells |
| | | NEUROD | |
| 112. | Embryonic | Foxn1 | Thymic epithelial cells |
| | fibroblasts | | |
| 113. | Fibroblasts | NF-κB and LEF-1 | Sweat gland cells |
| 114. | Amniotic fluid stem | OCT4 | Pluripotent stem cells |
| | cells | | |
| 115. | Cardiac | Klf4 and c-Myc | Adipocytes |
| | mesenchymal pro- | | |
| | genitors | | |
| 116. | Embryonic | Emx2, Hnf1b, Hnf4a, and Pax8 | Renal tubular |
| | fibroblasts, Adult | | epithelial cells |
| | tail-tip dermal fibro- | | |
| | blasts, Postnatal | | |
| | foreskin fibroblasts, | | |
| | and Fetal dermal | | |
| | fibroblasts | | |
| 117. | Endothelial | MYOCD | Smooth muscle cells |
| | progenitor cells | | |
| 118. | Embryonic stem | Cdx2, Arid3a, and Gata3 | Trophoblast stem cells |
| | cells | | |
| 119. | Embryonic | Dmrt1, Gata4, and Nr5a1 | Leydig cells |
| | fibroblasts and | | |
| | Adult tail-tip dermal | | |
| | fibroblasts | | |
| 120. | Postnatal dermal | ER71/ETV2 (ETS | Endothelial cells |
| | fibroblasts | variant 2) | |
| 121. | Dermal fibroblasts | PPARG2 | Adipocytes |
| 122. | Embryonic | Hnf4a, Foxa3, Gata6, and | Intestine progenitor |
| | fibroblasts and | Cdx2 | cells |
| | Umbilical vein | | |
| | endothelial cells | | |
| 123. | Embryonic | Myocd, Gata6, and Mef2c | Smooth muscle cells |
| | fibroblasts and | | |
| | Adult dermal | | |
| | fibroblasts | | |
| 124. | Epidermal cells | Foxc1 | Sweat gland cells |
| 125. | Renal proximal | SNAI2, EYA1, and SIX1 | Nephron progenitor |
| | tubular epithelial | | cells |
| | (HK2) cells | | |
| 126. | Fibroblasts (BJ and | HNF1A and Any two of the | Hepatocytes |
| | MRC-5 cells) | three factors: FOXA1, FOXA3, | |
| | | and HNF4A | |
| 127. | Non-sensory | Ascl1; Ascl1 and Neurod | Neuronal cells |
| | cochlear epithelial | | |
| | cells | | |
| 128. | Cardiac fibroblasts | Gata4, Mef2c, and Tbx5 | Cardiomyocytes |
| 129. | Astrocytes | Sox2 | Neural stem cells |
| 130. | Fetal and | Ascl1, Brn2, and Myt1l | Neuronal cells |
| | embryonic | | |
| | fibroblasts and | | |
| | Brain cells in vivo | | |
| 131. | Peripheral blood | CRX, RAX1, and NEUROD1 | Photoreceptor cells |
| | mono- nuclear cells | | |
| 132. | Gingival fibroblasts | OCT4, OSTERIX, and L-MYC | Osteoblasts |
| | and Adult dermal | | |
| | fibroblasts | | |
| 133. | Fibroblasts | OCT4, FOXA2, HNF1A, and | Hepatocytes |
| | | GATA3 | |
| 134. | Fibroblasts | SOX2, GATA3, and | Neurocytes |
| | | NEUROD1 | |
| 135. | Fibroblasts (3T6 | Ascl1, Brn2, and Foxa1 | Neuronal cells |
| | cells) | | |
| 136. | Mesenchymal stem | Hnf4a and Foxa3 | Hepatocytes |
| | cells | | |
| 137. | Dermal fibroblasts | ETV2 | Endothelial pro-genitor |
| | | | cells |
| 138. | Fibroblasts (3T6 | Ascl1, Brn4, and Tcf3 | Neuronal cells |
| | cells) | | |
| 139. | Mesenchymal stem | SOX2 | Neural stem cells |
| | cells and Dermal | | |
| | fibroblasts | | |
| 140. | Adult fibroblasts | SOX2; SOX2 and PAX6; | Neural precursor cells |
| | | SOX2 and LMX1A; SOX2, | |
| | | LMX1A, and FOXA2 | |
| 141. | Dermal fibroblasts | SOX2 and PAX6 | Neural precursor cells |
| 142. | Embryonic | Hnf4a and Foxa3 | Hepatocytes |
| | fibroblasts | | |
| 143. | Foreskin fibroblasts | ASCL1 + miR-124 + P53 | Neuronal cells |
| | | knock-down | |
| 144. | Bone marrow- | MSI1, NGN2, and MBD2 | Neural precursor cells |
| | derived cells, | | |
| | Fibroblasts, and | | |
| | Keratinocytes | | |
| 145. | Renal proximal | SNAI2, EYA1, and SIX1 | Nephron progenitor |
| | tubular epithelial | | cells |
| | (HK2) cells | | |
| 146. | Cardiac fibroblasts | miR-1, miR-133, miR-208, and | Cardiomyocytes |
| | | miR-499 | |
| 147. | Cardiac fibroblasts | Gata4, Mef2c, and Tbx5 + | Cardiomyocytes |
| | and Embryonic | miR-133; Gata4, Mef2c, Tbx5, | |
| | fibroblasts | Mesp1, and Myocd + miR-133 | |
| 148. | Fibroblasts | GATA4, MEF2C, TBX5, | Cardiomyocytes |
| | | ESRRG, MESP1, | |
| | | MYOCARDIN, ZFPM2, and | |
| | | HAND2 + miR-1 | |
| 149. | Adult fibroblasts | miR-9/9* and miR-124 | Neuronal cells |
| 150. | Adult fibroblasts | ISL1 and LHX3 + miR-9/9* and | Spinal cord motor |
| | | miR-124 | neurons |
| 151. | Brain vascular | ASCL1, MYT1L, BRN2, and | Cholinergic neuronal |
| | pericytes | TLX3 + miR-124 | cells |

The contacting may be performed by any of the methods described above (e.g., by transfection, electroporation, and/or the use of circRNA-LNP complexes).

In some embodiments, the cells are contacted with the circular RNA once. In some embodiments the cells are contacted with the circular RNA more than once, e.g., 2, 3, 4, 5, 6, 7, 8, 9, or 10 times. In some embodiments, the contacting is performed at effective intervals. The effective intervals may be, for example, once per day, once every other day, once every three days, once per week, once every two weeks, or once per month.

As explained above, the methods of directly converting a cell from a first cell type to a second cell type may comprise maintaining the cell under conditions under which the cell is converted to the second cell type. Such conditions are known to those of skill in the art, and may vary by cell type. As one example, after the cells have been contacted with one or more circular RNAs they can be cultured in standard media which is optionally supplemented with various reprogramming factors. The cells will be monitored to observe morphology, and the presence of markers characteristic of the second cell type.

Also provided herein are transdifferentiated cells produced using the methods described herein.

Also provided herein are compositions comprising a transdifferentiated cell, wherein the transdifferentiated cell comprises one or more exogenous circular RNAs encoding a transdifferentiation factor. In some embodiments, the transdifferentiation factor is any one of the transdifferentiation factors or combinations of transdifferentiation factors listed in Table 6. In some embodiments, the transdifferentiated cell is any one of the second cell types listed in Table 6. In some embodiments, the transdifferentiated cell is derived from a first cell type that is any one of the first cell types listed in Table 6.

Differentiation of iPSCs Using Circular RNAs

Also provided is an iPSC produced using the methods described herein. In some embodiments, the iPSC expresses one or more of Oct4, SOX2, Lin 28, SSEA4, SSEA3, TRA-1-81, TRA-1-60, CD9, Nanog, Fbxl5, Ecatl, Esgl, Eras, Gdf3, Fgf4, Cripto, Daxl, Zpf296, Slc2a3, Rexl, Utfl, and Nat1.

Also provided herein is a differentiated cell derived from an iPSC produced using the methods described herein. Methods for differentiating an iPSC are known to those of skill in the art. In some embodiments, the differentiated cell is a muscle cell, a neuron, a cardiomyocyte, a hepatocyte, an islet cell, a keratinocyte, a T-cell, or a NK-cell.

In some embodiments, an iPSC described herein (or an iPSC produced using a method that is not described herein) may be differentiated by contacting the iPSC with one or more circular RNAs encoding a differentiation factor. For example, in some embodiments, an iPSC is contacted with a circular RNA, or a DNA molecule encoding the same, which encodes a differentiation factor capable of differentiating the iPSC into a cell type of interest, such as a T-cell. In some embodiments, the differentiation factor is selected from RORA, HLF, MYB, KLF4, ERG, SOX4, LUC, HOXA9, HOXA10, and HOXA5. In some embodiments, the iPSC is contacted with at least one, at least two, at least three, at least four, at least five, at least six, at least seven, at least eight, at least nine, at least ten, or at least eleven circular RNAs, wherein each circular RNA encodes a differentiation factor selected from RORA, HLF, MYB, KLF4, ERG, SOX4, LUC, HOXA9, HOXA10, and HOXA5. In some embodiments, an iPSC is contacted with at least one, at least two, at least three, at least four, or at least five circular RNAs, wherein each circular RNA encodes a differentiation factor selected from HOXA9, ERG, RORA, SOX4, or MYB. In some embodiments, the iPSC is contacted with a plurality of circular RNAs, wherein each circular RNA encodes at least one of HOXA9, ERG, RORA, SOX4, or MYB. In some embodiments, the iPSC is contacted with at least one circular RNA, wherein the circRNA encodes one or more of the differentiation factors listed in Table 6. In some embodiments, the iPSC is additionally contacted with an EZH1 shRNA. The EXH1 shRNA expression may facilitate a switch from lineage restricted hematopoietic progenitors to progenitors with multi-lymphoid potential.

In some embodiments, an iPSC is differentiated into a CD34+CD38− cell. In some embodiments, contacting the iPSC with one or more of the circular RNAs encoding one or more of the following differentiation factors differentiates the iPSC into a CD34+CD38− cell: RORA, HLF, MYB, KLF4, ERG, SOX4, LUC, HOXA9, HOXA10, or HOXA5.

In some embodiments a CD34+CD45+ myeloid precursor cell is contacted with a circular RNA, or a DNA molecule encoding the same, that encodes one or more of RORA, HLF, MYB, KLF4, ERG, SOX4, LUC, HOXA9, HOXA10, or HOXA5. In some embodiments a CD34+CD45+ myeloid precursor cell is contacted with a circular RNA, or a DNA molecule encoding the same, that encodes one or more of HOXA9, ERG, RORA, SOX4, or MYB. In some embodiments, contacting with one or more circular RNAs iPSC as described above transdifferentiates the CD34+CD45+ cell into a CD34+CD38− cell. In some embodiments, the cells resulting after the contacting are self-renewing HSPCs (hematopoetic stem and progenitor cells) with erythroid and lymphoid potential.

In some embodiments, the iPSC produced using the methods described herein is younger as compared to an iPSC produced using traditional methods, such as use of a viral vector encoding a reprogramming factor or transfection of a linear RNA encoding a reprogramming factor. As described herein, “younger” refers to the fact that the cell is reprogrammed faster (i.e., within about 5, about 6, about 7, or about 8 days after transfection) as compared to traditional methods (i.e., about 9 days or more).

In some embodiments, the iPSC expresses different levels of one or more biomarkers as compared to an iPSC produced using traditional methods. For example, in some embodiments, the iPSC expresses lower levels of markers associated with cellular stress and/or cell death (apoptosis), as compared to an iPSC produced using traditional methods. For example, in some embodiments, the iPSC expresses lower levels of one or more heat shock proteins or caspases.

In some embodiments, the genome of the iPSC has different epigenetic modifications as compared to an iPSC produced using traditional methods. For example, in some embodiments, the iPSC may comprise altered levels of DNA methylations and/or histone modifications.

In some embodiments, a T-cell is contacted with one or more circular RNAs (or DNA molecules encoding the same) which encode factors that can improve the efficacy of the T-cell. In this context, improving the efficacy refers to promoting survival of the T-cell, and/or its anti-tumor activity when used in an immune-oncology setting. For example, the T-cell may be contacted with one or more circular RNAs that encode IL-12, IL-18, IL-15, or IL-7.

In some embodiments, a T-cell is contacted with one or more circular RNAs (or DNA molecules encoding the same) which improve the ability of the T-cell to home to a tumor tissue. For example, the T-cell may be contacted with one or more circular RNAs that encode CXCR2, CCR2B, or heparanase.

In some embodiments, a T-cell is contacted with one or more circular RNAs (or DNA molecules encoding the same) which help improve survival and/or promote the switch to a central memory phenotype. For example, the T-cell may be contacted with one or more circular RNAs that encode Suv39h1.

### Combination Methods for Reprogramming and Editing the Genome of a Cell

By combining methods for generating iPSCs with methods for genome editing thereof, the diagnostic and therapeutic power of iPSCs is enhanced. As used herein, the terms “genome editing” and “editing the genome” refer to modification of a specific locus of a nucleic acid (e.g., a DNA or an RNA) of a cell. Genome editing can correct pathology-causing genetic mutations derived from diseased patients and similarly can be used to induce specific mutations in disease-free wild-type cells (such as iPSCs). Accordingly, the instant disclosure provides combination methods for reprogramming and editing the genome of a cell. In some embodiments, the circular RNAs described herein may be used in methods for reprogramming and editing the genome of a cell.

Genome editing may comprise, for example, inducing a double stranded DNA break in the region of gene modification. In some embodiments, a locus of the DNA is replaced with an exogenous sequence by supplementation with a targeting vector. Any one of the following enzymes may be used to edit the DNA of a cell: a zinc-finger nuclease, a homing endonuclease, a TALEN (transcription activator-like effector nuclease), a NgAgo (argonaute endonuclease), a SGN (structure-guided endonuclease), a RGN (RNA-guided nuclease), or modified or truncated variants thereof. In some embodiments, the RNA-guided nuclease is an RNA-guided nuclease disclosed in any one of WO 2019/236566 (e.g., APG05083.1, APG07433.1, APG07513.1, APG08290.1, APG05459.1, APG04583.1, and APG1688.1 RNA-guided nucleases), WO 2021/030344 (e.g., APG05733.1, APG06207.1, APG01647.1, APG08032.1, APG05712.1, APG01658.1, APG06498.1, APG09106.1, APG09882.1, APG02675.1, APG01405.1, APG06250.1, APG06877.1, APG09053.1, APG04293.1, APG01308.1, APG06646.1, APG09748, and APG07433.1 RNA-guided nucleases), and WO 2020/139783 (APG00969, APG03128, APG09748, APG00771, APG02789, APG09106, APG02312, APG07386, APG09980, APG05840, APG05241, APG07280, APG09866, APG00868 RNA-guided nucleases), each of which is incorporated herein by reference in its entirety. In some embodiments, the RNA-guided nuclease is a Cas9 nuclease, a Cas12(a) nuclease (Cpf1), a Cas12b nuclease, a Cas12c nuclease, a TrpB-like nuclease, a Cas13a nuclease (C2c2), a Cas13b nuclease, a Cas 14 nuclease or modified or truncated variants thereof.

In some embodiments, a Cas9 nuclease is used to edit the genome of a cell. Cas9 is a large multifunctional protein having two putative nuclease domains, the HNH and RuvC-like. The HNH and the RuvC-like domains cleave the complementary 20-nucleotide sequence of the crRNA and the DNA strand opposite the complementary strand respectively. Several variants of the CRISPR-Cas9 system exists, and any one of these variants may be used in the methods disclosed herein: (1) The original CRISPR-Cas9 system functions by inducing DNA double-stranded breaks which are triggered by the wild-type Cas9 nuclease directed by a single RNA. (2) The nickase variant of Cas9 (D10A mutant) which is generated by the mutation of either the Cas9 HNH or the RuvC-like domain is directed by paired guide RNAs. (3) Engineered nuclease variant of Cas9 with enhanced specificity (eSpCas9). (4) Catalytically dead Cas9 (dCas9) variant is generated by mutating both domains (HNH and RUvC-like). dCas9, when merged with a transcriptional suppressor or activator can be used to modify transcription of endogenous genes (CRISPRa or CRISPRi) or when fused with fluorescent protein can be used to image genomic loci. (5) CRISPR-Cas9 fused with cytidine deaminase, results in a variant which induces the direct conversion of cytidine to uridine, hence circumventing the DNA double-stranded break. In some embodiments, the Cas9 nuclease is isolated or derived from S. pyogenes or S. aureus.

Cas9 requires a RNA guide sequence (“guide RNA” or “gRNA”) to target a specific locus. In some embodiments, the gRNA is a single-guide (“sgRNA”). The sgRNA may comprise a spacer sequence and a scaffold sequence. The spacer sequence is complementary to the target cleavage sequence, and directs the enzyme thereto. The scaffold region binds to the Cas9 enzyme.

Exemplary enzymes which may be used to edit the RNA of a cell include, but are not limited to, enzymes of the ADAR (adenosine deaminase acting on RNA) family. For example, the enzyme may be human ADAR1, ADAR2, or ADAR3, or a modified or truncated variant thereof. In some embodiments, the enzyme may be an ADAR from squid (e.g., Loligo pealeii) such as sqADAR2, or a modified or truncated variant thereof. In some embodiments, the enzyme may be an ADAR from C. elegans (e.g., ceADAR1 or ceADAR2) or D. melanogaster (e.g., dADAR), or a modified or truncated variant thereof.

In some embodiments, a method for reprogramming and editing the genome of a cell comprises contacting a cell with (i) a recombinant circular RNA comprising a protein-coding sequence, wherein the protein-coding sequence encodes at least one reprogramming factor, and (ii) an enzyme capable of editing the DNA or RNA of the cell.

In some embodiments, a method for reprogramming and editing the genome of a cell comprises contacting a cell with (i) a recombinant circular RNA comprising a protein-coding sequence, wherein the protein-coding sequence encodes at least one reprogramming factor, and (ii) a nucleic acid encoding an enzyme capable of editing the DNA or RNA of the cell.

In some embodiments, cell is contacted with the recombinant circular RNA before it is contacted with the enzyme or the nucleic acid encoding the same. In some embodiments, the cell is contacted with the recombinant circular RNA after it is contacted with the enzyme or the nucleic acid encoding the same. In some embodiments, the cell is contacted with the recombinant circular RNA at approximately the same time that it is contacted with the enzyme or the nucleic acid encoding the same.

In some embodiments, the methods for reprogramming and editing the genome of a cell further comprise contacting the cell with a nucleic acid encoding a guide RNA, or a guide RNA.

A composition for reprogramming and editing the genome of a cell may comprise, for example, a recombinant circular RNA (or a DNA molecule encoding the same) and an enzyme capable of editing DNA or RNA (or a DNA or RNA molecule encoding the same). In some embodiments, the recombinant circular RNA comprises a protein-coding sequence. In some embodiments, the circular RNA does not encode a protein. In some embodiments, the circular RNA is circBIRC6 (SEQ ID NO: 13), circCORO1C (SEQ ID NO: 14), or circMAN1A2 (SEQ ID NO: 15).

### Combination Methods for Transdifferentiating and Editing the Genome of a Cell

The circular RNAs described herein may be also be used in methods for transdifferentiating and editing the genome of a cell. Accordingly, provided herein are compositions and methods for transdifferentiating and editing the genome of a cell.

In some embodiments, a method for transdifferentiating and editing the genome of a cell comprises contacting a cell with (i) a recombinant circular RNA comprising a protein-coding sequence, wherein the protein-coding sequence encodes at least one transdifferentiation factor, and (ii) an enzyme capable of editing the DNA or RNA of the cell. In some embodiments, the transdifferentiation factor is selected from any of those listed in Table 6.

In some embodiments, a method for transdifferentiating and editing the genome of a cell comprises contacting a cell with (i) a recombinant circular RNA comprising a protein-coding sequence, wherein the protein-coding sequence encodes at least one transdifferentiation factor, and (ii) a nucleic acid encoding an enzyme capable of editing the DNA or RNA of the cell

The enzymes used to edit DNA or RNA in a method of transdifferentiating and editing the genome of a cell may be any of the enzymes listed above.

In some embodiments, cell is contacted with the recombinant circular RNA before it is contacted with the enzyme or the nucleic acid encoding the same. In some embodiments, the cell is contacted with the recombinant circular RNA after it is contacted with the enzyme or the nucleic acid encoding the same. In some embodiments, the cell is contacted with the recombinant circular RNA at approximately the same time that it is contacted with the enzyme or the nucleic acid encoding the same.

In some embodiments, the methods for transdifferentiating and editing the genome of a cell further comprise contacting the cell with a nucleic acid encoding a guide RNA, or a guide RNA.

A composition for transdifferentiating and editing the genome of a cell may comprise, for example, a recombinant circular RNA (or a DNA molecule encoding the same) and an enzyme capable of editing DNA or RNA (or a DNA or RNA molecule encoding the same). In some embodiments, the recombinant circular RNA comprises a protein-coding sequence. In some embodiments, the circular RNA does not encode a protein. In some embodiments, the circular RNA is circBIRC6 (SEQ ID NO: 13), circCORO1C (SEQ ID NO: 14), or circMAN1A2 (SEQ ID NO: 15). In some embodiments, the circular RNA encodes a reprogramming factor disclosed herein. In some embodiments, the circular RNA encodes one or more Oct3/4, Klf4, Sox2, Nanog, Lin28, c-Myc, and L-Myc. In some embodiments, the circular RNA encodes one or more of the transdifferentiation factors listed in Table 6.

### Additional Methods

As will be understood by those of skill in the art, the circular RNAs described herein, and related compositions, may be useful for one or more of the following methods.

In some embodiments, provided herein is a method reprogramming a cell which produces reduced cell death as compared to a method using linear RNA, the method comprising contacting a cell with a circular RNA, a complex, a vector, or a composition as described herein, and maintaining the cell under conditions under which the protein is expressed. In some embodiments, the reprogramming-induced cell death is reduced by at least 10%, at least 20%, at least 30%, at least 40%, at least 50%, at least 60%, at least 70%, at least 80%, at least 90%, at least 100%, at least 200%, at least 500% or more relative to a reprogramming method using linear RNA. In some embodiments, the cell is contacted with a combination of circular RNAs, wherein the combination of circular RNAs is selected from: (i) circOct3/4, circKlf4, circSox2, circNanog, circLin28, and circ c-Myc; (ii) circOct3/4, circKlf4, circSox2, circNanog, and circLin28; (iii) circOct3/4, circKlf4, circSox2, circNanog, circLin28, and circL-Myc; (iv) circOct3/4, circKlf4, circSox2, circNanog, and circLin28 (v) circOct3/4, circKlf4, circSox2, and circC-Myc; (vi) circOct3/4, circKlf4, circSox2, and circL-Myc; or (vii) circOct3/4, circKlf4, and circSox2. In some embodiments, the cell is contacted with circMyoD.

Also provided herein is a method of reducing time from reprogramming to picking, the method comprising contacting a cell with a circular RNA, a complex, a vector or a composition described herein, and maintaining the cell under conditions under which the protein is expressed, wherein the time from reprogramming to picking is reduced relative to a reprogramming method using linear RNA. As used herein, the term “picking” refers to manual selection if iPSC colonies by mechanical dissociation. In some embodiments, the time is reduced by at least 10%, at least 20%, at least 30%, at least 40%, at least 50%, at least 60%, at least 70%, at least 80%, at least 90%, at least 100%, at least 200%, at least 500% or more relative to a reprogramming method using linear RNA. In some embodiments, the cell is contacted with a combination of circular RNAs, wherein the combination of circular RNAs is selected from: (i) circOct3/4, circKlf4, circSox2, circNanog, circLin28, and circ c-Myc; (ii) circOct3/4, circKlf4, circSox2, circNanog, and circLin28; (iii) circOct3/4, circKlf4, circSox2, circNanog, circLin28, and circL-Myc; (iv) circOct3/4, circKlf4, circSox2, circNanog, and circLin28 (v) circOct3/4, circKlf4, circSox2, and circC-Myc; (vi) circOct3/4, circKlf4, circSox2, and circL-Myc; or (vii) circOct3/4, circKlf4, and circSox2. In some embodiments, the cell is contacted with circMyoD.

Also provided herein is a method of reducing the number of transfections induce to effect reprogramming of a cell, the method comprising contacting a cell with a circular RNA, a complex, a vector, or a composition described herein, and maintaining the cell under conditions under which the protein is expressed. In some embodiments, the number of transfections is reduced relative to a method using linear RNA. In some embodiments, the number of transfections to induce reprogramming of the cell is 1, 2, 3, 4, 5, 6, or 7. In some embodiments, the number of transfections is reduced by at least 10%, at least 20%, at least 30%, at least 40%, at least 50%, at least 60%, at least 70%, at least 80%, at least 90%, at least 100%, at least 200%, at least 500% or more relative to a method using linear RNA. In some embodiments, the cell is contacted with a combination of circular RNAs, wherein the combination of circular RNAs is selected from: (i) circOct3/4, circKlf4, circSox2, circNanog, circLin28, and circ c-Myc; (ii) circOct3/4, circKlf4, circSox2, circNanog, and circLin28; (iii) circOct3/4, circKlf4, circSox2, circNanog, circLin28, and circL-Myc; (iv) circOct3/4, circKlf4, circSox2, circNanog, and circLin28 (v) circOct3/4, circKlf4, circSox2, and circC-Myc; (vi) circOct3/4, circKlf4, circSox2, and circL-Myc; or (vii) circOct3/4, circKlf4, and circSox2. In some embodiments, the cell is contacted with circMyoD.

Also provided herein is method of increasing duration of protein expression in a cell, the method comprising contacting a cell with a circular RNA, a complex, a vector, or a composition described herein, and maintaining the cell under conditions under which the protein is expressed. In some embodiments, the duration of protein expression is increased relative to a method comprising transfection of the cell with a linear RNA encoding the same protein. In some embodiments, the duration of protein expression is increased by at least 10%, at least 20%, at least 30%, at least 40%, at least 50%, at least 60%, at least 70%, at least 80%, at least 90%, at least 100%, at least 200%, at least 500% or more relative to a method comprising transfection of the cell with a linear RNA encoding the same protein. In some embodiments, the duration of protein expression is increased by at least 1 hour, at least 4 hours, at least 8 hours, at least 12 hours, at least 1 day, at least 2 days, at least 3 days, at least 4 days, at least 5 days, at least 6 days, at least 1 week, at least 2 weeks, at least 3 weeks, or longer relative to a method comprising transfection of the cell with a linear RNA encoding the same protein. In some embodiments, the cell is contacted with a combination of circular RNAs, wherein the combination of circular RNAs is selected from: (i) circOct3/4, circKlf4, circSox2, circNanog, circLin28, and circ c-Myc; (ii) circOct3/4, circKlf4, circSox2, circNanog, and circLin28; (iii) circOct3/4, circKlf4, circSox2, circNanog, circLin28, and circL-Myc; (iv) circOct3/4, circKlf4, circSox2, circNanog, and circLin28 (v) circOct3/4, circKlf4, circSox2, and circC-Myc; (vi) circOct3/4, circKlf4, circSox2, and circL-Myc; or (vii) circOct3/4, circKlf4, and circSox2. In some embodiments, the cell is contacted with circMyoD.

Also provided herein is a method of improving cellular reprogramming efficiency, the method comprising contacting a cell with circular RNA, a complex, a vector, or a composition described herein, and maintaining the cell under conditions under which the protein is expressed, wherein the efficacy of cellular reprogramming is increased relative to a cellular reprogramming method in which linear RNA is used. In some embodiments, cellular reprogramming efficiency is increased by at least 10%, at least 20%, at least 30%, at least 40%, at least 50%, at least 60%, at least 70%, at least 80%, at least 90%, at least 100%, at least 200%, at least 500% or more relative to a method in which linear RNA is used. Enhanced cellular reprogramming efficiency may be observed based on qualitative and/or qualitative assessments including, but not limited to, reduced cell death, reduced immune response induced stress as measured by IFN-gamma secretion, reduced stress response gene induction. In some embodiments, the cell is contacted with a combination of circular RNAs, wherein the combination of circular RNAs is selected from: (i) circOct3/4, circKlf4, circSox2, circNanog, circLin28, and circ c-Myc; (ii) circOct3/4, circKlf4, circSox2, circNanog, and circLin28; (iii) circOct3/4, circKlf4, circSox2, circNanog, circLin28, and circL-Myc; (iv) circOct3/4, circKlf4, circSox2, circNanog, and circLin28 (v) circOct3/4, circKlf4, circSox2, and circC-Myc; (vi) circOct3/4, circKlf4, circSox2, and circL-Myc; or (vii) circOct3/4, circKlf4, and circSox2. In some embodiments, the cell is contacted with circMyoD.

Also provided herein is a method of increasing the number of reprogrammed cell colonies formed after reprogramming, the method comprising contacting a cell with circular RNA, a complex, a vector, or a composition, and maintaining the cell under conditions under which the protein is expressed, wherein the number of reprogrammed cell colonies formed after reprogramming is increased relative to a cellular reprogramming method in which linear RNA is used. In some embodiments, the number of reprogrammed cell colonies is increased by at least 10%, at least 20%, at least 30%, at least 40%, at least 50%, at least 60%, at least 70%, at least 80%, at least 90%, at least 100%, at least 200%, at least 500% or more relative to a method in which linear RNA is used. In some embodiments, the increased number of colonies may be observed about 7, about 8, about 9, about 10, about 11, about 12, about 13, about 14, about 15, about 16, about 17, about 18, about 19, or about 20 days post-transfection with one or more circRNAs encoding a transcription factor. In some embodiments, the cell is contacted with a combination of circular RNAs, wherein the combination of circular RNAs is selected from: (i) circOct3/4, circKlf4, circSox2, circNanog, circLin28, and circ c-Myc; (ii) circOct3/4, circKlf4, circSox2, circNanog, and circLin28; (iii) circOct3/4, circKlf4, circSox2, circNanog, circLin28, and circL-Myc; (iv) circOct3/4, circKlf4, circSox2, circNanog, and circLin28 (v) circOct3/4, circKlf4, circSox2, and circC-Myc; (vi) circOct3/4, circKlf4, circSox2, and circL-Myc; or (vii) circOct3/4, circKlf4, and circSox2. In some embodiments, the cell is contacted with circMyoD.

Also provided herein is a method of reprogramming cells in suspension, the method comprising contacting a cell in suspension with a circular RNA, a complex, a vector, or a composition described herein, and maintaining the cell under conditions under which the protein is expressed. In some embodiments, the cells express CD34 (i.e., they are CD34+). In some embodiments, the cell is contacted with a combination of circular RNAs, wherein the combination of circular RNAs is selected from: (i) circOct3/4, circKlf4, circSox2, circNanog, circLin28, and circ c-Myc; (ii) circOct3/4, circKlf4, circSox2, circNanog, and circLin28; (iii) circOct3/4, circKlf4, circSox2, circNanog, circLin28, and circL-Myc; (iv) circOct3/4, circKlf4, circSox2, circNanog, and circLin28 (v) circOct3/4, circKlf4, circSox2, and circC-Myc; (vi) circOct3/4, circKlf4, circSox2, and circL-Myc; or (vii) circOct3/4, circKlf4, and circSox2. In some embodiments, the cell is contacted with circMyoD.

Also provided herein is a method of improving morphological maturation of reprogrammed colonies, the method comprising contacting a cell in suspension with a circular RNA, a complex, a vector, or a composition described herein, and maintaining the cell under conditions under which the protein is expressed, wherein the morphological maturation is improved relative to a cellular reprogramming method in which linear RNA is used. Improved morphological maturation may include, for example, more tightly-packed colonies, colonies where more cells have a uniform shape and diameter, colonies comprising a clearly-defined border, and cells within iPSC colonies comprising a higher nuclear to cytoplasmic ratio and/or prominent nucleoli. In some embodiments, the morphological maturation of the reprogrammed colonies is improved by at least 10%, at least 20%, at least 30%, at least 40%, at least 50%, at least 60%, at least 70%, at least 80%, at least 90%, at least 100%, at least 200%, at least 500% or more relative to a method in which linear RNA is used. In some embodiments, the cell is contacted with a combination of circular RNAs, wherein the combination of circular RNAs is selected from: (i) circOct3/4, circKlf4, circSox2, circNanog, circLin28, and circ c-Myc; (ii) circOct3/4, circKlf4, circSox2, circNanog, and circLin28; (iii) circOct3/4, circKlf4, circSox2, circNanog, circLin28, and circL-Myc; (iv) circOct3/4, circKlf4, circSox2, circNanog, and circLin28 (v) circOct3/4, circKlf4, circSox2, and circC-Myc; (vi) circOct3/4, circKlf4, circSox2, and circL-Myc; or (vii) circOct3/4, circKlf4, and circSox2. In some embodiments, the cell is contacted with circMyoD.

Also provided herein is a suspension culture comprising one or more CD34-expressing cells, wherein the CD34-expressing cells comprise one or more exogenous circRNAs encoding a reprogramming factor. In some embodiments, the reprogramming factor is selected from Oct3/4, Klf4, Sox2, Nanog, Lin28, c-Myc, and L-Myc.

Also provided herein is a method for inducing a mesenchymal-to-epithelial transition (MET) of a somatic cell to an iPSC comprising contacting the somatic cell with one or more circular RNA encoding a reprogramming factor.

Also provided herein is a method for inducing a mesenchymal-to-epithelial transition (MET) of a somatic cell to an iPSC comprising contacting the somatic cell with one or more circular RNA encoding a reprogramming factor.

### Vectors, Compositions, and Cells

The instant disclosure also provides vectors comprising a nucleic acid (i.e., a DNA molecule) encoding a circular RNA as described herein. In some embodiments, the vector is a non-viral vector, such as a plasmid. In some embodiments, the vector is a viral vector. Examples of viral vectors include, but are not limited to, retroviral vectors, herpesvirus vectors, adenovirus vectors, adeno-associated virus (AAV) vectors, baculoviral vectors, alphavirus vectors, picornavirus vectors, vaccinia virus vectors, and lentiviral vectors. In some embodiments, the viral vector is a replication defective viral vector. Replication defective viral vectors retain their infective properties and enter cells in a similar manner as a replicating vectors, however once admitted to the cell a replication defective viral vector does not reproduce or multiply.

FIG. 4 provides a schematic of exemplary vector constructs that may be used to produce the circular RNAs described herein. In some embodiments, a nucleic acid encoding a circular RNA comprises a sequence encoding a reprogramming factor operably linked to an IRES. In some embodiments, a nucleic acid encoding a circular RNA comprises a sequence encoding a reprogramming factor operably linked to an IRES, flanked by a permuted Type I intron. In some embodiments, a nucleic acid encoding a circular RNA comprises a promoter and a sequence encoding a reprogramming factor operably linked to an IRES. In some embodiments, a nucleic acid encoding a circular RNA comprises a promoter and a sequence encoding a reprogramming factor operably linked to an IRES, flanked by a permuted Type I intron. In some embodiments, the nucleic acid further comprises an exon, or portion thereof.

Illustrative vector sequences that may be used to produce a circular RNA are shown in SEQ ID NO: 23-30. These vectors are referred to herein as circular RNA “precursors,” because they encode linear RNAs that, once transcribed, may be circularized to form circular RNA (i.e., the circular RNAs of SEQ ID NO: 30-38).

In some embodiments, a circular RNA precursor encodes a nGFP reprogramming factor. In some embodiments, the circular RNA precursor comprises the sequence of SEQ ID NO: 23, or a sequence at least 90%, at least 95%, at least 96%, at least 97%, at least 98%, or at least 99% identical thereto. In some embodiments, a circular RNA encodes a nGFP reprogramming factor. In some embodiments, the circular RNA comprises the sequence of SEQ ID NO: 31, or a sequence at least 90%, at least 95%, at least 96%, at least 97%, at least 98%, or at least 99% identical thereto.

In some embodiments, a circular RNA precursor encodes a MyoD reprogramming factor. In some embodiments, the circular RNA precursor comprises the sequence of SEQ ID NO: 24, or a sequence at least 90%, at least 95%, at least 96%, at least 97%, at least 98%, or at least 99% identical thereto. In some embodiments, a circular RNA encodes a MyoD reprogramming factor. In some embodiments, the circular RNA comprises the sequence of SEQ ID NO: 32, or a sequence at least 90%, at least 95%, at least 96%, at least 97%, at least 98%, or at least 99% identical thereto.

In some embodiments, a circular RNA precursor encodes an OCT4 reprogramming factor. In some embodiments, the circular RNA precursor comprises the sequence of SEQ ID NO: 25, or a sequence at least 90%, at least 95%, at least 96%, at least 97%, at least 98%, or at least 99% identical thereto. In some embodiments, a circular RNA encodes an OCT4 reprogramming factor. In some embodiments, the circular RNA comprises the sequence of SEQ ID NO: 33, or a sequence at least 90%, at least 95%, at least 96%, at least 97%, at least 98%, or at least 99% identical thereto.

In some embodiments, a circular RNA precursor encodes a SOX2 reprogramming factor. In some embodiments, the circular RNA precursor comprises the sequence of SEQ ID NO: 26, or a sequence at least 90%, at least 95%, at least 96%, at least 97%, at least 98%, or at least 99% identical thereto. In some embodiments, a circular RNA encodes a SOX2 reprogramming factor. In some embodiments, the circular RNA comprises the sequence of SEQ ID NO: 34, or a sequence at least 90%, at least 95%, at least 96%, at least 97%, at least 98%, or at least 99% identical thereto.

In some embodiments, a circular RNA precursor encodes a LIN28 reprogramming factor. In some embodiments, the circular RNA precursor comprises the sequence of SEQ ID NO: 27, or a sequence at least 90%, at least 95%, at least 96%, at least 97%, at least 98%, or at least 99% identical thereto. In some embodiments, a circular RNA encodes a LIN28 reprogramming factor. In some embodiments, the circular RNA comprises the sequence of SEQ ID NO: 35, or a sequence at least 90%, at least 95%, at least 96%, at least 97%, at least 98%, or at least 99% identical thereto.

In some embodiments, a circular RNA precursor encodes a NANOG reprogramming factor. In some embodiments, the circular RNA precursor comprises the sequence of SEQ ID NO: 28, or a sequence at least 90%, at least 95%, at least 96%, at least 97%, at least 98%, or at least 99% identical thereto. In some embodiments, a circular RNA encodes a NANOG reprogramming factor. In some embodiments, the circular RNA comprises the sequence of SEQ ID NO: 36, or a sequence at least 90%, at least 95%, at least 96%, at least 97%, at least 98%, or at least 99% identical thereto.

In some embodiments, a circular RNA precursor encodes a KLF4 reprogramming factor. In some embodiments, the circular RNA precursor comprises the sequence of SEQ ID NO: 29, or a sequence at least 90%, at least 95%, at least 96%, at least 97%, at least 98%, or at least 99% identical thereto. In some embodiments, a circular RNA encodes a KLF4 reprogramming factor. In some embodiments, the circular RNA comprises the sequence of SEQ ID NO: 37, or a sequence at least 90%, at least 95%, at least 96%, at least 97%, at least 98%, or at least 99% identical thereto.

In some embodiments, a circular RNA precursor encodes a cMYC reprogramming factor. In some embodiments, the circular RNA precursor comprises the sequence of SEQ ID NO: 30, or a sequence at least 90%, at least 95%, at least 96%, at least 97%, at least 98%, or at least 99% identical thereto. In some embodiments, a circular RNA encodes a cMYC reprogramming factor. In some embodiments, the circular RNA comprises the sequence of SEQ ID NO: 38, or a sequence at least 90%, at least 95%, at least 96%, at least 97%, at least 98%, or at least 99% identical thereto.

Also provided herein are compositions comprising a circular RNA or a vector as described herein. In some embodiments, a composition comprises (i) a circular RNA and (ii) a carrier or vehicle. In some embodiments, a composition comprises (i) a vector and (ii) a carrier or vehicle. Suitable carriers or vehicles include, for example, sterile water, sterile buffer solutions (e.g., solutions buffered with phosphate, citrate or acetate, etc.), sterile media, polyalkylene glycols, hydrogenated naphthalenes (e.g., biocompatible lactide polymers), lactide/glycolide copolymer or polyoxyethylene/polyoxypropylene copolymers. In some embodiments, the carrier or vehicle may comprise lactose, mannitol, substances for covalent attachment of polymers such as polyethylene glycol, complexation with metal ions or inclusion of materials in or on particular preparations of polymer compounds such as polylactate, polyglycolic acid, hydrogel or on liposomes, microemulsions, micelles, unilamellar or multilamellar vesicles, erythrocyte fragments or spheroplasts. In some embodiments, the pH of the carrier or vehicle is in the range of 5.0 to 8.0, such as in the range of about 6.0 to about 7.0. In some embodiments, the carrier or vehicle comprises salt components (e.g., sodium chloride, potassium chloride), or other components which render the solution, for example, isotonic. Further, the carrier or vehicle may comprise additional components such as fetal calf serum, growth factors, human serum albumin (HSA), polysorbate 80, sugars or amino acids.

Also provided herein are cells comprising a recombinant circular RNA, a vector, or a composition as described herein. In some embodiments, the cell is a prokaryotic cell. In some embodiments, the cell is a eukaryotic cell. In some embodiments, the cell is a mammalian cell (e.g., a murine, bovine, simian, porcine, equine, ovine, or human cell). In some embodiments, the cell is a human cell.

### Kits

Kits for expressing a protein in a cell are also provided. In some embodiments, the kit comprises at least one circular RNA as described herein, or a vector comprising a nucleic acid (i.e., a DNA molecule) encoding the same. In some embodiments, the kit comprises a vessel containing a circular RNA or a DNA molecule encoding the same. In some embodiments, the kit comprises a plurality of vessels, wherein each vessel comprises a circular RNA or a DNA molecule encoding the same. In some embodiments, a kit comprises a vessel comprising a plurality of circular RNA molecules, wherein each circular RNA molecule comprises a sequence encoding a protein. In some embodiments, a kit comprises a vessel comprising a plurality of DNA molecules, wherein each DNA molecule encodes a circular RNA molecule that can be used to express a protein in a cell. In some embodiments, the kit also comprises a set of instructions for using the at least one circular RNA (or DNA molecule encoding the same) for expressing a protein in a cell.

In some embodiments, a kit comprises one or more circular RNAs, or DNA molecules encoding the same, wherein each circular RNA, or DNA molecule encoding the circular RNA, comprises a sequence that encodes at least one protein. In some embodiments, the kit may further comprise a circular RNA that does not encode any protein or miRNA, or a DNA molecule encoding the same. In some embodiments, the kit may further comprise a circular RNA that encodes a miRNA, or a DNA molecule encoding the same. In some embodiments, the kit may comprise a single vessel containing each of: (i) the one or more circular RNAs, or DNA molecules encoding the same, wherein each circular RNA (or DNA sequence) encodes a protein, (ii) optionally, a circular RNA, or DNA molecule encoding the same, that does not encode any protein or miRNA, (iii) optionally, a circular RNA that encodes a miRNA, or a DNA molecule encoding the same. In some embodiments, the kit may comprise a plurality of vessels, wherein each vessel comprises one of: (i) at least one circular RNA or DNA molecule encoding the same, that encodes a protein, (ii) optionally, a circular RNA, or DNA molecule encoding the same, that does not encode any protein or miRNA, (iii) optionally, a circular RNA that encodes a miRNA, or a DNA molecule encoding the same. In some embodiments, the kit also comprises a set of instructions for using the at least one circular RNA (or DNA sequence encoding the same) for expressing a protein in a cell.

In some embodiments, a kit for reprogramming somatic cells and/or generating iPSCs is provided. In some embodiments, the kit comprises at least one circular RNA encoding a reprogramming factor (e.g., a transcription factor), or a vector comprising a nucleic acid (i.e., a DNA molecule) encoding the same In some embodiments, the kit comprises a vessel containing a circular RNA or a DNA molecule encoding the same. In some embodiments, the kit comprises a plurality of vessels, wherein each vessel comprises a circular RNA or a DNA molecule encoding the same. In some embodiments, a kit comprises a vessel comprising a plurality of circular RNA molecules, wherein each circular RNA molecule comprises a sequence encoding a transcription factor. In some embodiments, a kit comprises a vessel comprising a plurality of DNA molecules, wherein each DNA molecule encodes a circular RNA molecule that can be used to express a transcription factor in a cell. In some embodiments, the kit also comprises a set of instructions for using the at least one circular RNA for reprogramming somatic cells and/or generating iPSCs.

In some embodiments, a kit comprises one or more circular RNAs, or DNA molecules encoding the same, wherein each circular RNA, or DNA molecule encoding the circular RNA, comprises a sequence that encodes at least one reprogramming factor. The reprogramming factors may be, for example, any one of the reprogramming factors listed in Table 1. In some embodiments, the kit may further comprise a circular RNA that does not encode any protein or miRNA, or a DNA molecule encoding the same. In some embodiments, the kit may further comprise a circular RNA that encodes a miRNA, or a DNA molecule encoding the same. In some embodiments, the kit may comprise a single vessel containing each of: (i) the one or more circular RNAs, or DNA molecules encoding the same, wherein each circular RNA (or DNA sequence) encodes a reprogramming factor, (ii) optionally, a circular RNA, or DNA molecule encoding the same, that does not encode any protein or miRNA, (iii) optionally, a circular RNA that encodes a miRNA, or a DNA molecule encoding the same. In some embodiments, the kit may comprise a plurality of vessels, wherein each vessel comprises one of: (i) at least one circular RNA or DNA molecule encoding the same, that encodes a reprogramming factor, (ii) optionally, a circular RNA, or DNA molecule encoding the same, that does not encode any protein or miRNA, (iii) optionally, a circular RNA that encodes a miRNA, or a DNA molecule encoding the same. In some embodiments, the kit also comprises a set of instructions for using the at least one circular RNA (or DNA sequence encoding the same) for expressing a reprogramming factor in a cell.

In some embodiments, a kit for transdifferentiating cells is provided. In some embodiments, the kit comprises at least one circular RNA encoding a reprogramming factor (e.g., a transcription factor), or a vector comprising a nucleic acid (i.e., a DNA molecule) encoding the same. In some embodiments, the kit comprises a vessel containing a circular RNA or a DNA molecule encoding the same. In some embodiments, the kit comprises a plurality of vessels, wherein each vessel comprises a circular RNA or a DNA molecule encoding the same. In some embodiments, a kit comprises a vessel comprising a plurality of circular RNA molecules, wherein each circular RNA molecule comprises a sequence encoding a transdifferentiation factor. In some embodiments, a kit comprises a vessel comprising a plurality of DNA molecules, wherein each DNA molecule encodes a circular RNA molecule that can be used to express a transdifferentiation factor in a cell. In some embodiments, the kit also comprises a set of instructions for using the at least one circular RNA for transdifferentiating cells.

In some embodiments, a kit comprises one or more circular RNAs, or DNA molecules encoding the same, wherein each circular RNA, or DNA molecule encoding the circular RNA, comprises a sequence that encodes at least one transdifferentiation factor. The transdifferentiation factors may be, for example, any one of the transdifferentiation factors listed in Table 6. In some embodiments, the kit may further comprise a circular RNA that does not encode any protein or miRNA, or a DNA molecule encoding the same. In some embodiments, the kit may further comprise a circular RNA that encodes a miRNA, or a DNA molecule encoding the same. In some embodiments, the kit may comprise a single vessel containing each of: (i) the one or more circular RNAs, or DNA molecules encoding the same, wherein each circular RNA (or DNA sequence) encodes a transdifferentiation factor, (ii) optionally, a circular RNA, or DNA molecule encoding the same, that does not encode any protein or miRNA, (iii) optionally, a circular RNA that encodes a miRNA, or a DNA molecule encoding the same. In some embodiments, the kit may comprise a plurality of vessels, wherein each vessel comprises one of: (i) at least one circular RNA or DNA molecule encoding the same, that encodes a transdifferentiation factor, (ii) optionally, a circular RNA, or DNA molecule encoding the same, that does not encode any protein or miRNA, (iii) optionally, a circular RNA that encodes a miRNA, or a DNA molecule encoding the same. In some embodiments, the kit also comprises a set of instructions for using the at least one circular RNA (or DNA sequence encoding the same) for expressing a transdifferentiation factor in a cell.

In some embodiments, a kit comprises a plurality of circular RNAs (or DNA molecules encoding the same), wherein each circular RNA encodes a reprogramming factor selected from Oct3/4, Klf4, Sox2, Nanog, Lin28, c-Myc, and L-Myc. Each of the circular RNAs (or DNA molecules encoding the same) may be provided in separate vessels, or may be provided in a single vessel.

In some embodiments, a kit comprises a plurality of circular RNAs (or DNA molecules encoding the same), wherein each of circular RNA encodes a reprogramming factor selected from Oct3/4, Sox2, and Klf4. Each of the circular RNAs (or DNA molecules encoding the same) may be provided in separate vessels, or may be provided in a single vessel.

In some embodiments, a kit comprises a plurality of circular RNAs (or DNA molecules encoding the same), wherein each of circular RNA encodes a reprogramming factor selected from Oct3/4, Sox2, c-Myc, and Klf4. Each of the circular RNAs (or DNA molecules encoding the same) may be provided in separate vessels, or may be provided in a single vessel.

In some embodiments, a kit comprises a plurality of circular RNAs (or DNA molecules encoding the same), wherein each of circular RNA encodes a reprogramming factor selected from Oct3/4, Sox2, L-Myc, and Klf4. Each of the circular RNAs (or DNA molecules encoding the same) may be provided in separate vessels, or may be provided in a single vessel.

In some embodiments, a kit may comprise a linear RNA cable of being circularized, or a DNA sequence encoding the same. In some embodiments, a kit may further comprise one or more reagents for circularizing a linear RNA, such as an RNA or DNA ligase, or Mg2+ and guanosine 5′ triphosphate (GTP).

In some embodiments, a kit comprises: (i) a vessel comprising a circular RNA encoding OCT4 and a buffer (e.g., 1-10 mM sodium citrate, pH 6.5); (ii) a vessel comprising a circular RNA encoding SOX2 and a buffer (e.g., 1-10 mM sodium citrate, pH 6.5); (iii) a vessel comprising a cirRNA encoding KLF4 and a buffer (e.g., 1-10 mM sodium citrate, pH 6.5); and (iv) packaging and instructions therefor. The kit may further comprise a vessel comprising a circular RNA encoding c-MYC or L-MYC and a buffer (e.g., 1-10 mM sodium citrate, pH 6.5); a vessel comprising a cirRNA encoding LIN28 and a buffer (e.g., 1-10 mM sodium citrate, pH 6.5); a vessel comprising a cirRNA encoding NANOG and a buffer (e.g., 1-10 mM sodium citrate, pH 6.5); or a combination thereof.

In some embodiments, a kit comprises: (i): (a) the circular RNA reprogramming factor(s) of any one or more circularized reprogramming factor combinations listed in Table 2, wherein each factor is contained individually in a separate vessel or wherein two or more of such factors are combined together in a single or plurality of vessels; and/or (b) the circular RNA(s) of any one or more combinations of circular RNAs for generating iPSCs listed in Table 3, wherein each such circular RNA is contained individually in a separate vessel or wherein two or more of such circular RNAs are combined together in a single or plurality of vessels; and (ii) packaging and instructions therefor.

In some embodiments, a kit comprises: (i): (a) the circular RNA reprogramming factor(s) of any one or more circularized reprogramming factor combinations listed in Table 2, wherein each factor is contained individually in a separate vessel or wherein two or more of such factors are combined together in a single or plurality of vessels; and/or (b) the circular RNA(s) of any one or more combinations of circular RNAs for generating iPSCs listed in Table 3, wherein each such circular RNA is contained individually in a separate vessel or wherein two or more of such circular RNAs are combined together in a single or plurality of vessels; and wherein for either one of (i)(a) and (i)(b), the circularized reprogramming factors and/or the circular RNAs of Table 2 and Table 3, respectively, are suspended in a buffer; and (iii) packaging and instructions therefor.

In any of the kits described above, the circular RNA or DNA molecule encoding the same may be provided in a composition that further comprises a buffer. The buffer may comprise, for example 1-10 mM sodium citrate. In some embodiments, the pH of the buffer is in the range of about 2 to about 12, such as about 6.5.

### EXAMPLES

The following examples, which are included herein for illustration purposes only, are not intended to be limiting.

### Example 1: Generation of Circular RNAs and Linear mRNAs

Circular RNA expression vectors were generated comprising an RNA sequence encoding circOct3/4 (SEQ ID NO: 1), circKlf4 (SEQ ID NO: 2, 3), circSox2 (SEQ ID NO: 4), circNanog (SEQ ID NO: 5, 6), circLin28 (SEQ ID NO: 7), circC-Myc (SEQ ID NO: 8, 9), or circL-Myc (SEQ ID NO: 10-12). Additional expression vectors are generated encoding circBIRC6 (SEQ ID NO: 13), circCORO1C (SEQ ID NO: 14), or circMAN1A2 (SEQ ID NO: 15). Circular RNA expression vectors encoding circnGFP or circmCherry were produced for use as reporters.

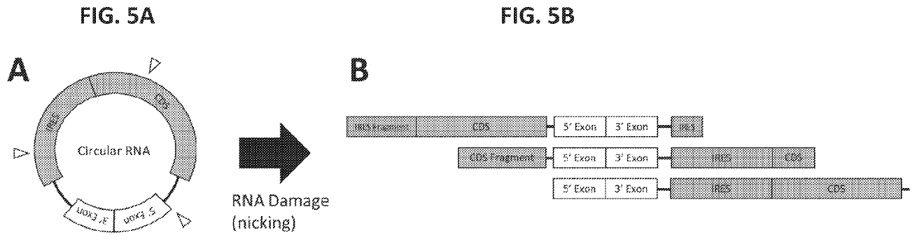

The general protocol for circular RNA production is illustrated in FIG. 4. A permuted-intron exon (PIE) circRNA construct comprises the 3′ intron and exon fragment of a group I ribozyme followed by a sequence of interest (e.g., an internal-ribosomal entry site (IRES) and coding sequence (CDS) for a desired protein product) followed by the 5′ exon fragment and 5′ intron. The PIE construct was cloned into an appropriate plasmid to allow amplification and plasmid DNA purification. Plasmid DNA was linearized with a restriction enzyme and used as the template for in-vitro transcription of a precursor RNA. The 5′ and 3′ ends of the precursor RNA fold together via long-range base pairing and tertiary structural interactions to form a ribozyme. In the presence of Mg2+ and free guanosine (for instance guanosine 5′ triphosphate (GTP)) the ribozyme spontaneously splices the exon fragments via sequential transesterification reactions forming a circular RNA and releasing the introns. Additional heating or other manipulation can dissociate the intron halves. Nicking of the circular RNA can lead to formation of re-linearized nicked circRNA degradation products. This is illustrated in FIG. 5.

Construct Design and Synthesis: Plasmids containing the desired circularization constructs were purchased from a gene synthesis vendor. The circularization constructs comprise a T7 promoter followed by sequences corresponding to the 3′ half of a permuted ribozyme (consisting of the 3′ intron, 3′ exon fragment and flanking sequences) followed by the sequence of interest (IRES and gene of interest), followed by the 5′ half of the permuted ribozyme (flanking sequences, 5′ exon fragment and 5′ intron) and a restriction site for plasmid linearization.

Plasmid linearization: Plasmid (typically 20 μg) was linearized by incubation for 1 hour with an appropriate restriction enzyme in a reaction mixture prepared according to the product insert (Thermo Scientific: Fast Digest Eco32I or MssI), the resulting reactions were cleaned up on a silica-based spin column (Thermo Scientific: GeneJET PCR Purification Kit) according to the product insert.

In vitro transcription: Linearized plasmid was used as the template for in-vitro transcription of the precursor RNAs for circularization. Exemplary precursor RNA sequences are provided in SEQ ID NOs: 23-30, described below in Table 7. The in-vitro transcription reactions were prepared as indicated in the product insert (Invitrogen MEGAscript T7 Transcription Kit), incubated for 2 to 3 hours at 37 degrees C. after which DNase (Invitrogen Turbo DNase) was added to the reaction (DNase was added at a ratio of 4 units of DNase per μg of template DNA), mixed, and incubated for an additional 30 minutes at 37 C.

TABLE 7
Exemplary precursor RNAs

| Reference ID | Encoded Gene | SEQ ID NO: |
|---|---|---|
| nGFP_precursor | nGFP | 23 |
| MyoD_precursor | MyoD | 24 |
| OCT4_precursor | OCT4 | 25 |
| SOX2_precursor | SOX2 | 26 |
| LIN28_precursor | LIN28 | 27 |
| NANOG_precursor | NANOG | 28 |
| KLF4_precursor | KLF4 | 29 |
| cMYC_precursor | cMYC | 30 |

Post-transcriptional RNA clean-up and circularization: 200 μg of IVT precursor RNA product in was prepared in a final concentration of 2 mM GTP (guanosine triphosphate) and 10 mM Mg2+ in a total volume of 100 μL. The reaction mixture was incubated at 55 C for 15 minutes and then immediately cleaned up with the MEGAClear Transcription cleanup kit. Eluted RNA was collected and quantified with a Nanodrop One operating in RNA mode.

Size-Exclusion Chromatography: Circular RNA was purified from other circularization byproducts via size-exclusion chromatography. 50 to 500 μg of post-transcriptionally circularized RNA product was injected on an FPLC system configured with appropriate SEC column(s) and fractions corresponding to peak circular RNA concentration were collected and pooled. The mobile phase was TE pH 6. Pooled fractions were concentrated and buffer exchanged into 10 mM Tris pH 7.4 using a centrifugal MWCO filter (Amicon Ultra 0.5 mL 100K MWCO, Millipore-Sigma). Resulting RNA was then quantified with a Nanodrop One operating in RNA mode.

Peak circular RNA fractions were identified by running 50 to 600 ng of RNA from a given fraction on a 2% agarose gel (Thermo Scientific 2% EX Gel) to visualize the relative intensity of bands associated with circular RNA or other circularization byproducts. Peak fractions were identified via visual inspection or quantification of band intensity using the ImageLab software package (Bio-Rad).

Phosphatase Treatment: SEC purified RNA was prepared in a reaction mixture at a ratio of 1 U alkaline phosphatase per μg of RNA per product insert (Thermo Scientific: FastAP) and incubated at 37 degrees C. for 1 hour. The reaction mixture was then cleaned up with a silica-column based kit (GeneJET RNA Cleanup and Concentration Micro Kit, Thermo Scientific). RNA was eluted in TE pH 6 and stored at −20 degrees C. until use

Exemplary sequences of circularized RNAs are provided in SEQ ID NOs: 31-38 and detailed below in Table 8.

| Reference ID | Encoded Gene | SEQ ID NO: |
|---|---|---|
| nGFP_circRNA | nGFP | 31 |
| MyoD_circRNA | MyoD | 32 |
| OCT4_circRNA | OCT4 | 33 |
| SOX2_circRNA | SOX2 | 34 |
| LIN28_circRNA | LIN28 | 35 |
| NANOG_circRNA | NANOG | 36 |
| KLF4_circRNA | KLF4 | 37 |
| cMYC_circRNA | cMYC | 38 |

Linear RNA vectors for producing linear RNAs encoding reporter genes (nGFP or mCherry) were also produced by Trilink. Linear RNA is generated by IVT using either modified or unmodified nucleotide triphosphates (NTPs). Before or during IVT, a 5′ cap and a poly A tail may be added. Linear RNAs made using modified NTPs are referred to herein as “modified linear RNAs” and linear RNAs are made using unmodified NTPs are referred to herein as “unmodified linear RNAs.”

### Example 2: Characterization of Circular RNA

Experiments were performed to further characterize the circular RNAs generated in Example 1.

### PIE-Based Circular RNA Production is Dependent on Autocatalytic Splicing Activity of a Permuted Group I Intron

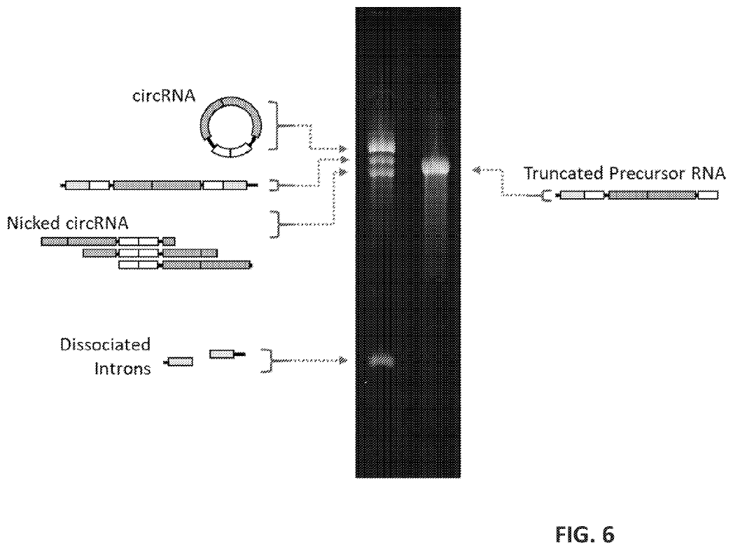

FIG. 6 shows agarose gel electrophoresis of in vitro transcription products (100 ng) from a DNA template corresponding to either a full-length (WT) or truncated (ΔSS) permuted intron-exon (PIE) precursor RNA. The full-length precursor RNA is co-transcriptionally circularized leading to the formation of circular RNA, nicked circular RNA, and the excised intron halves. The 3′ truncated precursor RNA (ΔSS) lacks the permuted 5′ intron and splice site and is unable to circularize, resulting in a single RNA product.

The precursor RNA band was identified by comparing its known length with a ssRNA ladder (not shown) and the known length of the truncated precursor RNA product. Similarly, the nicked circular RNA and intron bands were identified by comparison of their known lengths with the ladder and relative position on the gel.

Circular RNA is known to migrate more slowly (at a higher apparent molecular weight) than linear RNA of the same size when separated on a 2% agarose gel (See Wesselhoeft et al., Nat Commun 9, 2629 (2018). https://doi.org/10.1038/s41467-018-05096-6), allowing identification of the remaining band as circular RNA.

### Verification of RNA Circularization

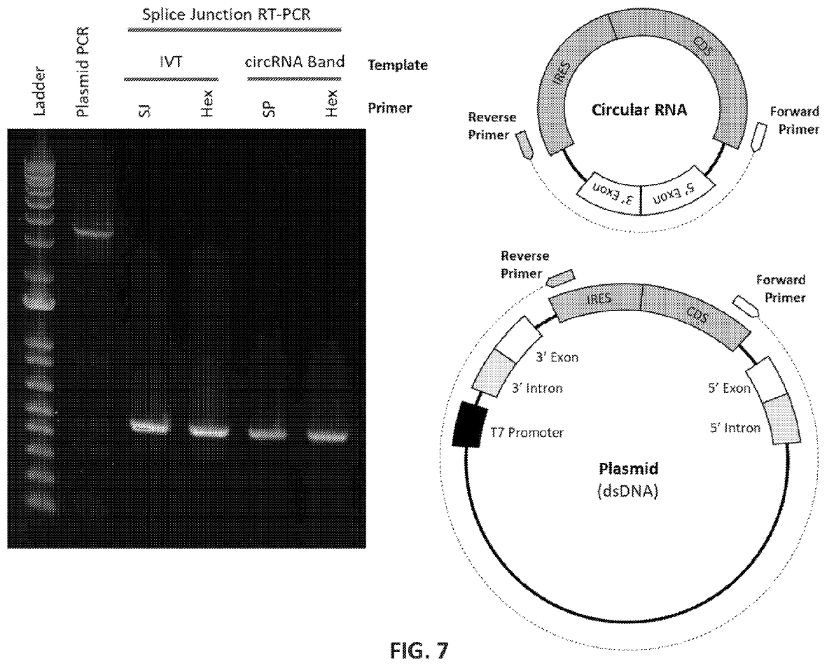

FIG. 7 provides splice junction-specific RT-PCR analysis to verify that the circRNA band contains circularized RNA. Both the IVT product and gel-purified circRNA band were used as templates for first-strand cDNA synthesis using either random-hexamer (Hex) or a splice junction (SJ) specific primer. The resulting cDNA was used as a template for PCR amplification using a forward and reverse primer pair spanning the splice junction expected to form upon circularization.

RT-PCR with all combinations of RNA template and first-strand cDNA primer produced the expected 507 nucleotide PCR product (lanes 3-6). Formation of the splice junction was confirmed by Sanger sequencing of the PCR products (not shown).

As a control, the same PCR primers were used to amplify DNA from the plasmid containing the circRNA PIE construct. As the plasmid contains no splice junction the primers face “outwards” from either end of the PIE construct and DNA polymerase must traverse the backbone of the plasmid to produce an amplicon. The resulting amplified product corresponded to the 3,594 base-pair expected product (lane 2).

### Circular RNA Purification and Characterization

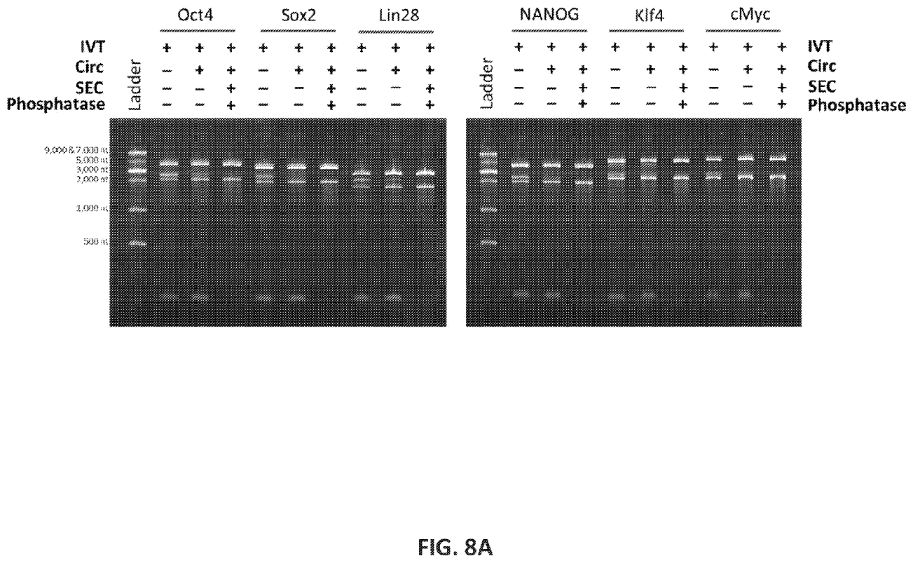

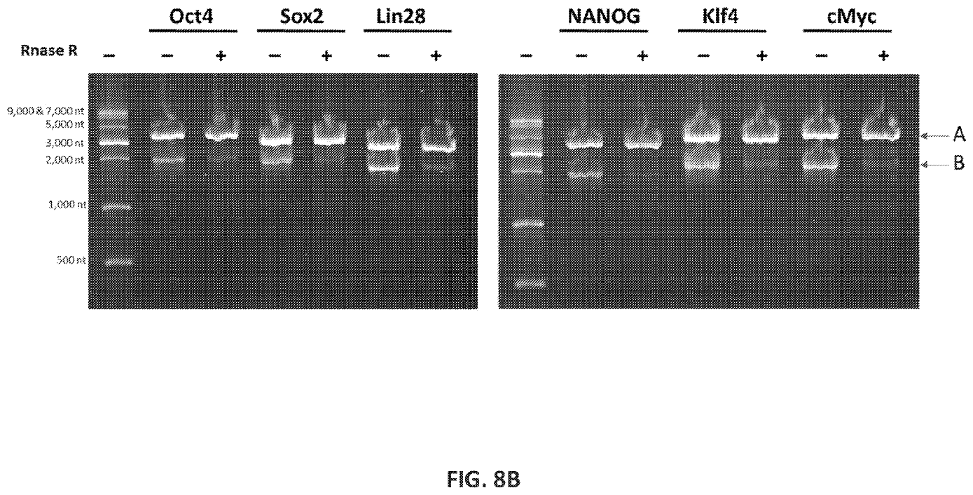

During circRNA production the initial in vitro transcription and co-transcriptional circularization products (IVT) were subjected to an additional post-transcriptional circularization step (Circ) and separated via size-exclusion chromatography (SEC). Selected SEC fractions were then pooled and treated with phosphatase prior to transfection. FIG. 8A shows the distribution of RNA species remaining after each indicated step for each of the six reprogramming factors. Notably, most of the precursor RNA remaining after the in vitro transcription reaction is consumed by the post-transcriptional circularization step which leads to both additional circRNA formation and circRNA nicking. The SEC step is effective at removing the high and low molecular weight by-products but has a more modest ability to purify circRNA from linearized nicked circRNA.

RNase R is a 3′→5′ processive exonuclease that digests linear RNA. Circular RNA lacks a 3′ end and is therefore expected to be protected from RNAse degradation. SEC fractions containing both putative circular and putative nicked-circular RNA were selected and incubated with and without RNase R to confirm the identity of circRNA and linear contaminant products. The resulting products were then separated by agarose gel electrophoresis. As shown in FIG. 8B, the more slowly migrating (A, circular RNA) band in each lane was observed to be resistant to Rnase R digestion, whereas the more quickly migrating band (B, linear RNA) was susceptible.

### Example 3: Using Circular RNAs for Protein Expression

The circular RNAs from Example 1 were used to express proteins in fibroblasts. The stability of protein expression from circular RNAs, modified linear mRNAs, and unmodified linear mRNAs were compared.

Human dermal fibroblasts (HDFs) were seeded at a density of 50K/well in a 24 well plate and grown for about 24 hours in Cascade 106 media containing low-serum growth supplement. Cells were transfected with 30 ng RNA (Linear mRNA from TriLink or CircRNA) linear and circular RNAs encoding Oct4, Klf4, Sox2, cMyc, Nanong, Lin28 using the RNAiMax reagent according to manufacturer's instructions. Cultures were fixed 24 hours later and processed for immunofluorescent chemistry (IFC) using antibodies specific to the protein of interest. Images were acquired on a Nikon Ti2 inverted microscope and captured using a high resolution PCO sCMOS camera. Additional experiments are performed in which circular RNAs are conjugated to lipid nanoparticles (“LNPs”) to form circRNA-LNP complexes. The circRNA-LNP complexes may then be used to directly introduce the circular RNA into the cell, without the need for any transfection reagent.

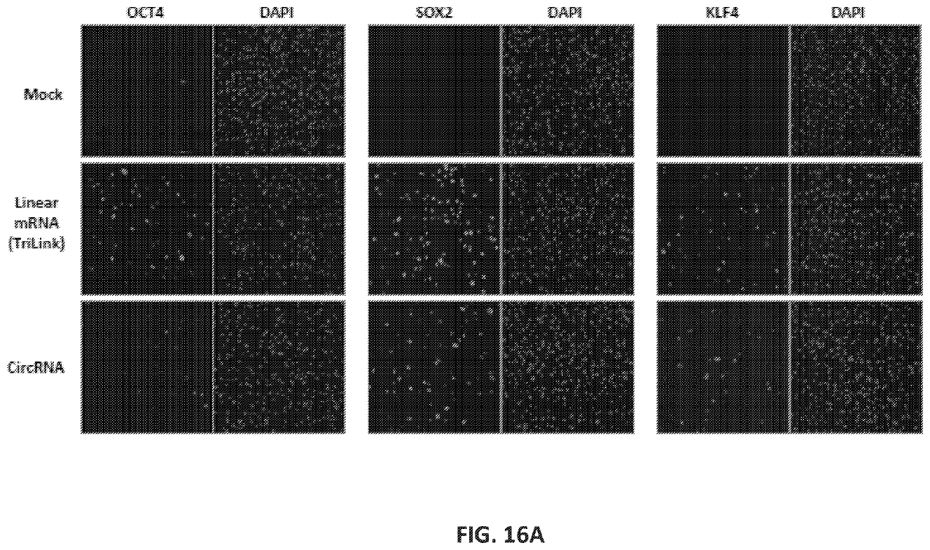

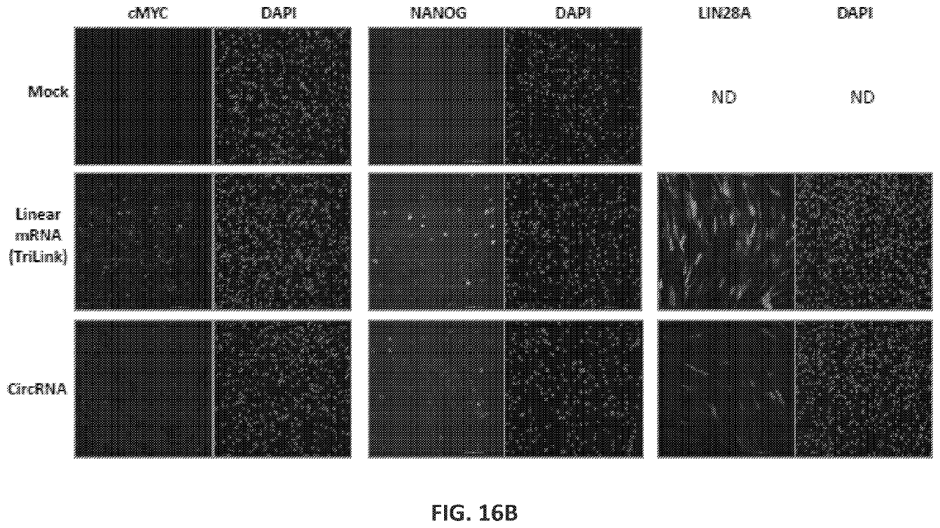

Results are provided in FIG. 16A and FIG. 16B. All reprogramming factors used were transcription factors and demonstrated nuclear localization almost exclusively (stained using DAPI). LIN28A is an RNA-binding protein that is predominantly cytosolic. As shown, circRNA constructs resulted in protein expression in transduced fibroblasts. Note that protein expression levels are generally lower from circRNA than from linear mRNA. Interestingly, as shown in fibroblast reprogramming experiments, circRNA cocktail of reprogramming factors gave rise to more iPSC colonies compared to linear mRNA cocktail. Without bound by any theory, it is believed lower, but more sustained expression of reprogramming factors is more conducive to reprogramming than high, short-duration expression

### Example 4: Testing the Immunogenicity of circRNA and circRNA-LNP Complexes

The immunogenicity of circular RNA and circRNA-LNP complexes is compared to that of modified and unmodified linear mRNAs.

Briefly, circular RNAs, circRNA-LNP complexes, modified linear mRNAs, or unmodified linear mRNAs are introduced into cells. At various time points, the expression levels of interferon-regulated genes (e.g., one or more of the genes described at www.interferome.org) are examined using qPCR and/or ELISA, according to a standard protocol. In some experiments, the circRNAs or linear mRNAs are introduced into cells in combination with B18R, optionally in combination with additional immune evasion factors such as E3 and K3. The B18R and additional immune evasion factors are provided in the form of linear mRNA, circular RNA, or are directly added to the media as proteins.

To determine whether the circRNAs and/or linear mRNAs affect cell viability, cell viability is monitored after the RNAs are introduced into the cells. Specifically, kinetics of cell growth/viability are tracked from 24 hours to 10 days post introduction of RNA into the cell. Cell viability is also measured after single or multiple transfections.

### Example 5: Generation of iPSCs Using circRNA Reprogramming of Adherent Cells

Experiments were performed to compare reprogramming of fibroblasts to iPSCs using non-modified linear mRNAs and circular RNAs encoding various reprogramming factors.

The experimental groups were as follows:
(a) Group 1—Mock—no RNA
(b) Group 2—RepoCell's Stemgent StemRNA 3^{rd} Gen reprogramming kit for human fibroblasts (non-modified linear mRNA)
(c) Group 3—Non-modified linear mRNA synthesized by Trilink
(d) Group 4—Non-modified circular RNA

For each group, 3 RNA cocktails encoding reprogramming factors, Vaccina virus immune suppression proteins, and miRNA mimics were combined and aliquoted for the desired number of transfections (Human Gene Therapy, 26(11), DOI: 10.1089/hum.2015.045). The RNA cocktails are as follows:
(a) Reprogramming factor mRNA cocktail comprising mRNAs encoding Oct4, Sox2, Klf4, Lin28, cMyc, and Nanog (OSKLMN)—RNA present at a 3:1:1:1:1:1 molar ratio.
(b) Vaccinia immune evasion mRNA cocktail comprising mRNAs encoding E3, K3, and B18R (EKB)
(c) A microRNA mimic cocktail comprising mimics of miR302a, miR302b, miR302c, miR302d and miR367.

For Group 4 (the circular RNA group), linear mRNA was used for the Vaccina EKB gene cocktail. A small amount of RNA encoding nGFP was spiked into each group to help visualize RNA delivery into cells. Linear nGFP mRNA was used for group 2 and group 3 and circular nGFP RNA was used for group 4. MicroRNA mimic were purchased from Dharmacon. The Repocell Stemgent kit was used as an overall reprogramming control. The RNA constructs in groups 3 and 4 have identical ORF sequences for each reprogramming factor. Therefore, the linear mRNAs in Group 3 are a direct control for the circular RNAs in Group 4.

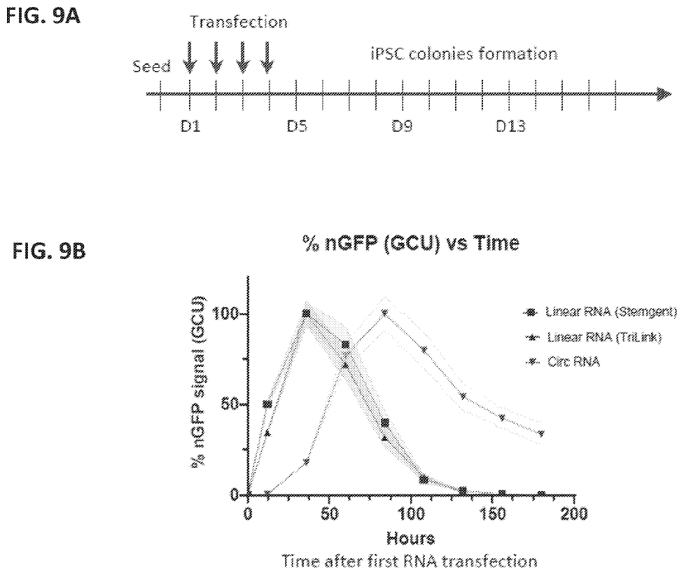

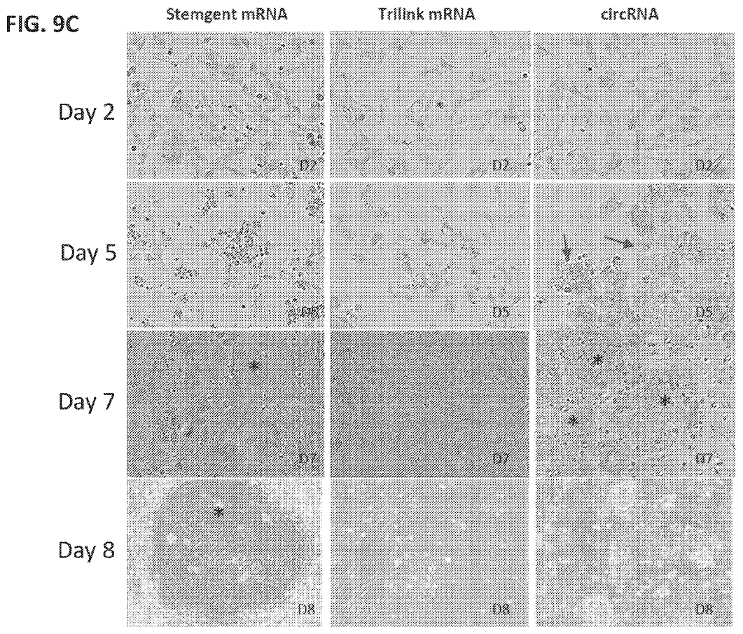

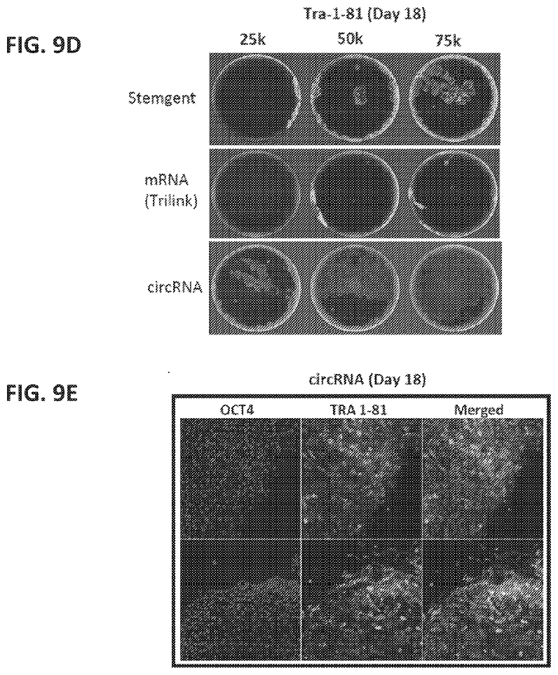

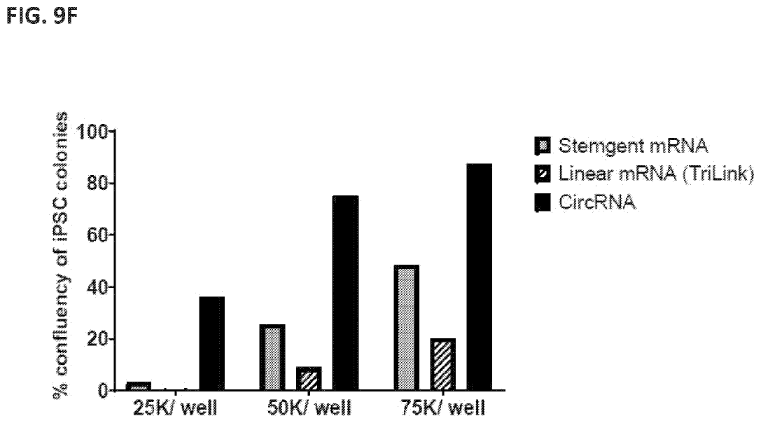

Human dermal fibroblasts (HDF) were plated at 3 different densities—25,000 cells/well, 50,000 cells/well, and 75,000 cells/well—in 6-well plates. On day 1, fibroblast media was replaced with Nutristem-hPSC-XF media. Transfections were performed using the RNAiMax lipofectamine reagent as per manufacturer's instructions. Cells were grown under hypoxia (5% O₂, 5% CO₂ at 37 C) until the end of the experiment. Three additional transfections were performed on Days 2, 3, and 4 (See schematic in FIG. 9A). Fibroblast reprogramming was conducted in iMatrix-511-coated 6-well plates in Nutristem media, under hypoxia, from day 1 to day 16/18, when iPSC colonies were manually picked. On day 16 or 18, selected colonies were picked into 24-well plates coated with vitronectin, and the iPSC culture media was changed to E8. Individual iPSC clones were continued to be expanded in E8 media, and passaged using Versene.

Cells were imaged and examined for phenotypic changes (e.g. survival, mesenchymal to epithelial transition (MET) at early stages of reprogramming, as well as acquisition of pluripotent stem cell (PSC)-like characteristics like high nuclear to cytoplasmic ratio, and colony formation).

By day 16, when PSC-like colonies were large enough (bearing few thousand cells per colony), 3 to 10 colonies were manually scored under a dissecting microscope and picked for further expansion and characterization. Reprogramming plates were fixed on day 18 and processed for IFC. Costaining with anti-OCT4 and anti-TRA 1-81 was performed along with DAPI and imaged using the Nikon Ti2 microscope to acquire high resolution images. Plates were also imaged in Incucyte to capture whole-well images.

A timeline for reprogramming HDFs using linear and circular RNA is provided in FIG. 9A. HDFs were seeding at three densities (25 k, 50 k and 75 k per well in 6-well plates) on day 0, followed by four daily transfections. iPSC colonies formed and emerged around day 8˜10.

A small amount of nGFP RNA was included in the daily transfection cocktails to monitor RNA delivery into the fibroblasts. Trilink mRNA encoding nGFP was included in both Stemgent mRNA cocktail and Trilink mRNA cocktail, while circRNA encoding nGFP was included in the circRNA cocktail. IncuCyte was used to image reprogramming cultures and measure nGFP protein expression daily. FIG. 9B shows nGFP expression normalized as the percentage of the peak expression. nGFP protein encoded by circRNA showed prolonged expression (slower turnover) compared to nGFP protein encoded by linear mRNA.

The mesenchymal to epithelial transition (MET), characteristic morphological change during iPSC reprogramming, was observed in all three experimental groups (StemgentmRNA, TrilinkmRNA, and circRNA). However, circRNA transfected cultures exhibited accelerated morphological transition from fibroblast-like cells to clusters of polygonal cells (arrows) followed by a transition to clusters of densely packed cells resembling early iPSC colonies (asterisks), compared to linear mRNA groups (FIG. 9C).

FIG. 9D shows whole well images of day 18 reprogramming cultures stained with the pluripotency marker, Tra-1-81. Green denotes areas with Tra-1-81+ cells and are presumed to represent iPSCs. circRNA transfected cultures gave rise to significantly more Tra-1-81-positive areas than wells transfected with either Stemgent mRNA or Trilink mRNA, suggesting circRNA provided increased reprogramming efficiency (i.e., resulted in more pluripotent cells on day 18 of reprogramming compared to the linear mRNA methods). mRNA reprogramming using Stemgent kit resulted in higher reprogramming efficiency than mRNA reprogramming using Trilink mRNA. Trilink mRNA-derived iPSCs only emerged at the edges of the wells. FIG. 9E provides representative images of circRNA reprogramming iPSCs on day 18 of culture and stained for Tra-1-81 and Oct4 expression. Results are quantified in FIG. 9F. Briefly, reprogramming was quantified for each reprogramming condition on day 18 using IncuCyte. Each well was analyzed for the area covered by iPSC colonies, based on morphology in phase images, as percent confluency of the well. For all the seeding densities (25 k, 50 k and 75 k), circRNA reprogrammed wells produced the largest areas covered by iPSC colonies, compared to Stemgent mRNA or Trilink mRNA reprogrammed wells, suggesting highest reprogramming efficiency by circRNA.

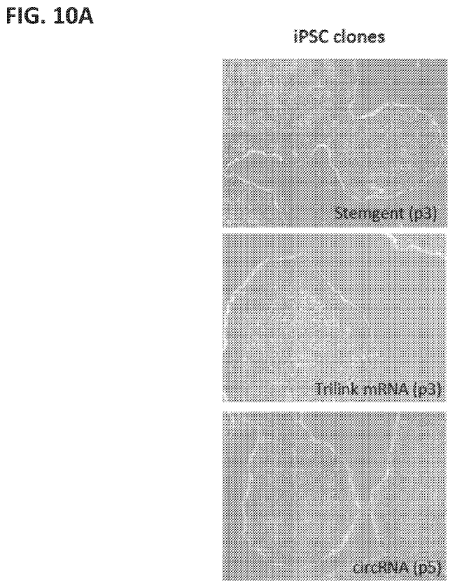

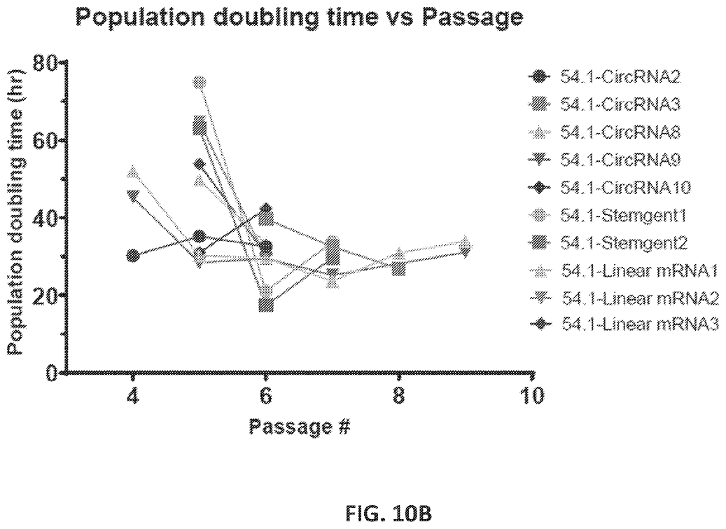

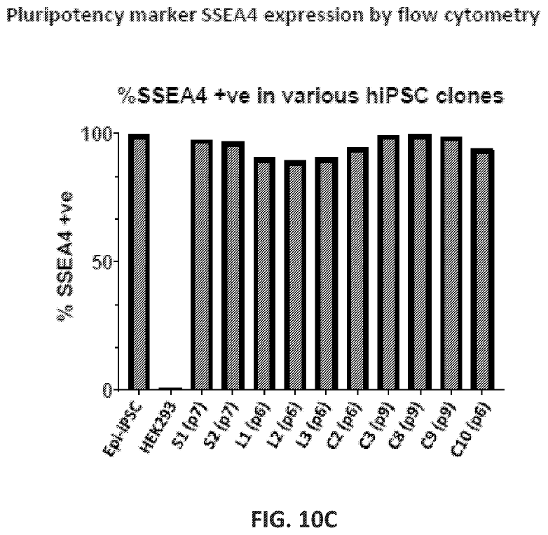

Additional read-outs were performed to further characterize the iPSCs derived from circRNA reprogramming. FIG. 10A shows representative images of iPSCs derived from Stemgent mRNA reprogramming kit (top), mRNA synthesized from Trilink (middle), and circRNA (bottom), from cultures between passage 3 and 5. Each of these iPSC clones exhibited characteristic iPSC morphology. FIG. 10B shows population doubling time (PDT) for iPSCs derived from RNA reprogramming. The growth rate of earlier passage iPSC clones (i.e., before passage 6) is dynamic, often reflected in fluctuating population doubling time. After passage 6 doubling time for most clones stabilized and remained around 30 hrs, which is in the range of typical iPSC doubling time. FIG. 10C shows expression of the pluripotency marker, SSEA4, in iPSC clones (at early passages-P6 to P9) derived from different RNA reprogramming cocktails. Clones S1 and S2 were derived from Stemgent kit. Clones L1, L2 and L3 were derived from Trilink linear mRNAs. Clones C2, C3, C8, C9 and C10 were derived from circRNA. All clones exhibited ≥90% SSEA4+ cells in the population. The Epi-iPSC line was used as positive control, while HEK293 cells were the negative control. Additional experiments are performed to assess the expression of OCT4. These assays confirm that iPSCs reprogrammed with circRNA demonstrate similar morphological, growth, and expression characteristics as iPSCs reprogrammed with linear mRNA.

The above experiments demonstrate that protein expression during reprogramming is prolonged with circRNA (based on nGFP expression, See FIG. 9B) and that the kinetics of MET are accelerated with circRNA (FIG. 9C). Overall, more iPSC colonies were generated with circRNA, i.e. reprogramming with circRNA demonstrated a higher reprogramming efficiency compared to methods with linear mRNA (FIG. 9D). Further, iPSCs derived from circRNA exhibit consistent expansion and express pluripotency markers (FIG. 10).

Additional experiments are performed to assess gene expression patterns, epigenetics, and tri-lineage differentiation. Clones of interest are expanded, frozen, and stored in liquid nitrogen for later use.

### Example 6: Optimized Reprogramming Protocols with Circular RNA

Experiments were performed to determine the optimal reprogramming protocols for adherent cells. Experimental groups were established with reduced numbers of transfections and the absence or absence of the Vaccinia EKB immune evasion cocktail. The RNAs encoding the reprogramming factors were the same as those described in Example 5:
(a) Mock—no RNA
(b) ReproCell's Stemgent StemRNA 3^{rd} Gen Reprogramming Kit for Human Fibroblasts (non-modified mRNA)
(c) Non-modified mRNA synthesized by Trilink
(d) Non-modified circRNA.

In each RNA group, 4 transfection conditions were tested:
(a) 4 transfections (4 Tx, +EKB cocktail) (standard)—days 1, 2, 3, 4 post-seeding
(b) 2 transfections (2 Tx, +EKB cocktail)—days 1 and day 3 post-seeding
(c) 1 transfection (1 Tx, +EKB cocktail)—day 1 post seeding
(d) 4 transfections without EKB cocktail (4 Tx −EKB cocktail)—days 1, 2, 3, 4 post-seeding.

Each transfection includes 3 cocktails (except the −EKB condition, which included only (a) and (b))
(a) Reprogramming factor mRNA cocktail OSKLMN (Oct4/Sox2/Klf4/Lin28/cMyc/Nanog)
(b) microRNA mimic cocktail,
(c) Vaccinia immune evasion mRNA cocktail EKB (E3/K3/B18R).

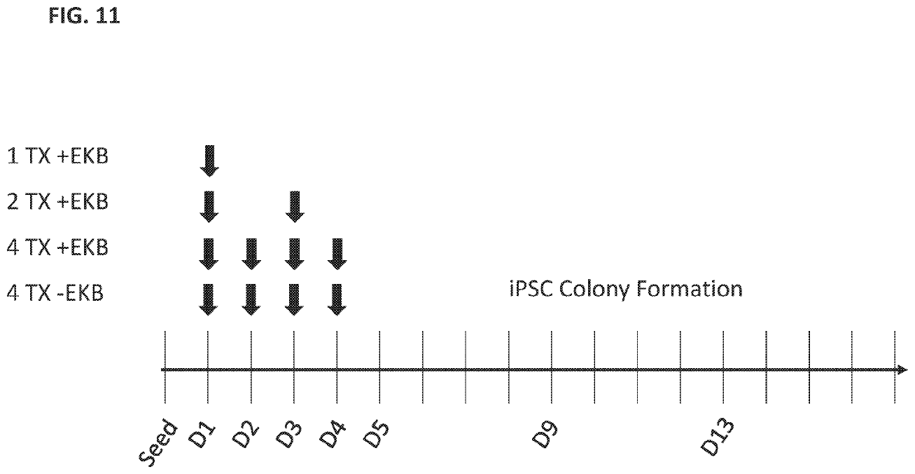

Transfections were performed according to the methods outlined in Example 4. A schematic of the transfection schedule is provided in FIG. 11.

FIG. 12 shows the morphological progression for the cultures in each experimental group. The circRNA-transfected subgroup in the 4 Tx +EKB group (FIG. 12A) shows iPSC colony-like morphology as early as day 5 and hundreds of colonies by day 9. In contrast, Stemgent and Trilink linear RNA conditions do not show iPSC colony-like morphology until day 7 and have merely tens of colonies on day 9. FIG. 12B shows morphological progressions during reprogramming for the 4 Tx −EKB group. FIG. 12C shows morphological progressions during reprogramming for the 2 Tx group. Insets in the circRNA-transfected condition show iPSC colony-like morphology as early as day 5 and hundreds of colonies by day 9. FIG. 12D shows morphological progressions during reprogramming for the 1 Tx group. Images were acquired with a 4× objective to capture the largest field of view possible. No iPSC colony observed from any group with 1 transfection.

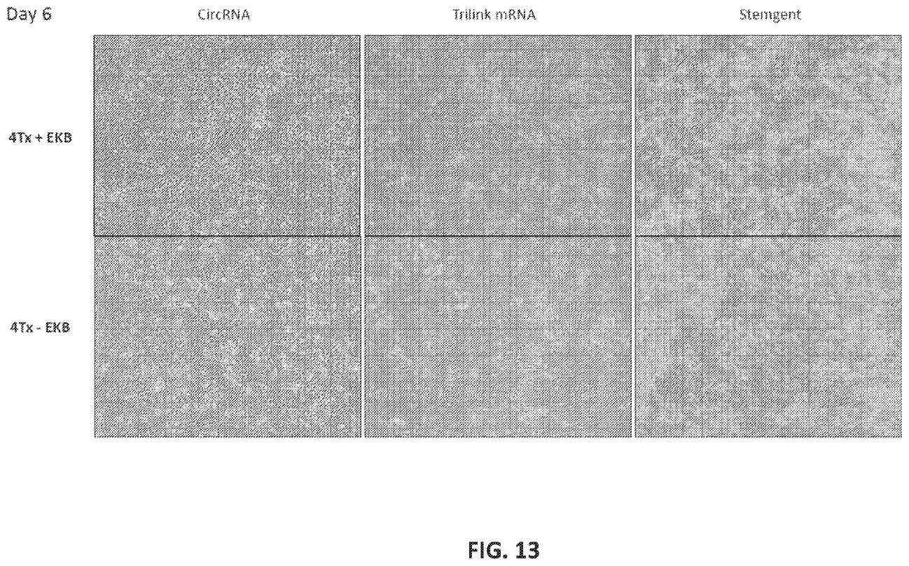

Further analysis was performed on the two 4×transfection groups (4Tx with and without the EKB cocktail). Images were acquired on day 6 of culture (2 days after the fourth and final transfection) and assayed for cell toxicity based on the number of rounded dead cells in the cultures (FIG. 13). circRNA cultures had very few rounded, light-reflective cells regardless of the presence or absence of EKB, indicative of low cell toxicity. In contrast, both Trilink mRNA and Stemgent kit resulted in large numbers of rounded or floating cells in the cultures suggesting toxicity. In addition, the morphological mesenchymal-to-epithelial transition (MET) is much more pronounced in circRNA cultures at this early stage than in Trilink and Stemgent cultures (Trilink showed the least amount of MET, still exhibiting spindly fibroblast morphology). Based on these data, circRNA transfection and reprogramming resulted in less cell death than mRNA transfection/reprogramming, thereby demonstrating lower toxicity during early reprogramming (i.e., during active transfection days). See FIG. 13.

Reprogramming efficiency was determined by a semi-quantitative analysis of Tra-1-81/Oct4 staining of day 16 cultures. On day 16 of reprogramming, cultures were fixed and stained with Tra-1-81 and Oct4, and whole-well images were scanned using IncuCyte (FIG. 14). Tra-1-81 and Oct4 double positive areas are presumed iPSC colonies. None of the RNA types successfully gave rise to iPSC colonies after only 1 transfection (left panel). For 2Tx+EKB, 4Tx+EKB, and 4Tx−EKB transfection conditions, circRNA-transfected cultures resulted in greatest amount of Tra-1-81/Oct4-double positive areas compared to Stemgent or Trilink mRNA. This was true regardless of seeding density (25 k, 50 k or 75 k), suggesting the highest reprogramming efficiency from circRNA.

Reprogramming efficiency for each experimental group are summarized in Table 9 below.

TABLE 9
Reprogramming Efficiency at Day 16

| | | RNA Type | | |
|---|---|---|---|---|
| Transfection | Seeding | | Trilink | Stemgent |
| Condition | Density | circRNA | mRNA | mRNA |
| 1 Tx + EKB | 25k | (−) | (−) | (−) |
| | 50k | (−) | (−) | (−) |
| | 75k | ND | (−) | (−) |
| 2 Txs + EKB | 25k | (++) | (−) | (+) |
| | 50k | (+++) | (−/+) | (+) |
| | 75k | (+++) | (−/+) | (+) |
| 4 Txs + EKB | 25k | (++) | (−/+) | (+) |
| | 50k | (+++) | (+) | ND |
| | 75k | (+++) | (+) | (−/+) |
| 4 Txs − EKB | 25k | (++) | (−/+) | (+) |
| | 50k | (+++) | (+) | (++) |
| | 75k | (+++) | (+) | (++) |

ND—No data available
(−) no iPSC colonies observed
(+), (++), (+++) denote increasing levels of reprogramming efficiency, with +++ being the highest, most efficient

As illustrated in Table 9, fibroblast reprogramming with circRNAs resulted in increased reprogramming efficiency regardless of the experimental transfection protocol used and independent of initial fibroblast seeding density. Results are further quantified in FIG. 14B. Reprogramming was quantified for each reprogramming condition on day 16 using IncuCyte. Each well was analyzed for the area covered by iPSC colonies, based on morphology in phase images, as percent confluency of the well. For all the transfection conditions except 1Tx+EKB, circRNA produced the largest areas covered by iPSC colonies (i.e., the most iPSC colonies), compared to Stemgent mRNA or Trilink mRNA. None of the RNA types successfully gave rise to iPSC colonies after only 1 transfection. Among different transfection conditions, biggest difference between circRNA vs. mRNA was seen in 2Tx+EKB (2 transfections of circRNA cocktails were able to produce large numbers of iPSC colonies, while 2 transfections of Stemgent or Trilink mRNA were not).

In sum, circRNA-based reprogramming is more efficient at reprogramming fibroblasts compared to linear mRNA methods. circRNA reprogramming demonstrated lower toxicity during early reprogramming (during active transfection days, FIG. 13), resulted in the generation of more iPSC-like colonies at earlier timepoints (FIG. 12), resulted in similar or greater numbers of iPSC-like colonies with fewer starting cells compared to linear mRNA (Table 9 and FIG. 14), resulted in faster rate of reprogramming (colony formation as early as day 5 or day 6, FIG. 12), and resulted in quicker colony maturation (based on morphology, FIG. 13).

### Example 7: Delivery of circRNA to CD34+ Cells

CD34+ suspension culture cells cannot be successfully reprogrammed with traditional methods because the low efficiency of these methods renders repeated transfection a requirement, however, this is toxic to the cells. The results presented in the previous examples demonstrate that circular RNA reprogramming is more efficient and results in significantly less cell death than traditional methods. Accordingly, it was hypothesized that reprogramming of CD34+ cells might be possible with circRNAs. Experiments were performed to determine the optimal method to deliver linear and circular RNA into CD34+ hematopoietic stem cells. The transfection efficiency of nGFP RNA (linear and circular) using Neon electroporation system and liposome-based reagents were evaluated.

Purified CD34+ cells were transfected with linear or circular RNA (nGFP), using one of the three transfection methods below and nGFP protein expression was evaluated after RNA transfection:
(a) Neon nucleofection (using Neon Transfection System by ThermoFisher)
(b) Lipofectamine RNAiMAX reagent (reagent used for transfecting fibroblasts)
(c) DOTAP Liposomal transfection reagent (MilliporeSigma)

Nucleofection resulted in a transfection efficiency of 80˜100% (most cells received nGFP, regardless of mRNA or circRNA). RNAiMAX transfection resulted in very low transfection efficiency. DOTAP transfection resulted in cell clumping and no transfection.

### Example 8: Generation of iPSCs Using circRNA Reprogramming of Suspension Cells

Suspension cells (such as CD34+ cells) are reprogrammed using circRNA, to generate iPSCs. Briefly, purified CD34+ cells (Hemacare) are expanded for 3 days (‘days −3 to 0’) in hematopoietic stem cell (HSC) media containing a cocktail of 5 cytokines (100 ng/mL each of SCF, TPO, FLT3-L, IL3, and IL6)

On day 0 (3 days post-expansion), 100K cells are combined with RNA cocktails for reprogramming and electroporated using the Neon electroporator. Electroporated cells are transferred to 0.5 mL of SCGM media with cytokines (100 ng/mL each of SCF, TPO, FLT3-L, IL3, and IL6) in non-adherent wells of a 24 well plate. Cells are allowed to recover for approximately 48 hours before either transferring to VTN-coated 6 well plates on d3 or transfected for a second time.

Transfected cells cultured on VTN-coated wells are gradually transitioned to pluripotent stem cell (PSC) medium as follows:
(a) On days 4 and 6, 1 ml of spent medium is replaced with 1 mL of “minus” medium (HSC medium without cytokines)
(b) On day 7, 1 mL of spent medium from each is replaced with 1 mL of PSC medium
(c) On days 8-18, spent media in wells is replaced with 100% PSC culture media
(d) Putative iPSC clones are expected to emerge on day 12-18

In some experiments, the cells are also contacted with circB18R, optionally in combination with additional immune evasion factors such as E3 and K3. In some experiments, the cells are also contacted with circBIRC6, circCORO1C, or circMAN1A2.

Morphological progression of the cells towards a pluripotent state is tracked, and reprogramming efficiencies are quantified. iPSC clones are selected and characterized. Specifically, pluripotency marker expression is analyzed (using human embryonic stem cells (hES) or iPSCs as a control), along with gene expression patterns, epigenetics, and tri-lineage differentiation. Clones of interest are expanded, frozen, and stored in liquid nitrogen for later use.

### Example 9: Use of a Circular RNA Encoding MyoD to Induce Muscle Cell Differentiation

Transducing MyoD in fibroblasts (non-muscle cells) has been shown to be sufficient to cause them to transdifferentiate into myoblasts (muscle cells). In this example, circRNAs encoding MyoD were used to generate muscle cells.

Briefly, human dermal fibroblasts (HDFs) were plated at 3 different densities-25K, 50K or 75K per well in 6-well plates on day 0 in 10% Fibroblast Expansion Media (FEM). On day 1, 10% FEM was supplemented with 200 ng/ml B18R recombinant protein. Cells were grown under normoxia (and 5% CO2 at 37 C) until the end of the experiment. Cells were transfected daily for 6 days with 50 ng MyoD-encoding circRNAs or linear RNA (Trilink) using RNAiMAX. Media was changed approximately 16 hours post transfection with 10% FEM containing 200 ng/ml B18R protein. 10% FEM media was changed daily and cells were imaged and examined for phenotype changes (e.g. survival, multinucleated myotube formation). Following the final transfection on day 6, media was changed to 2% FEM containing 200 ng/ml B18R protein starting on day 7.

Reprogramming plates were fixed on day 12 and processed for IFC. Costaining with antibodies specific to Desmin, Myosin heavy chain (MHC), and Myogenin (MYOG), was performed along with DAPI and imaged using the Nikon Ti2 microscope.

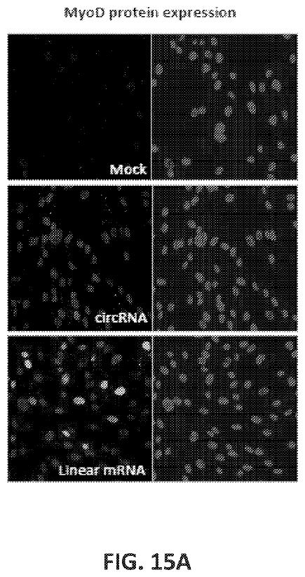

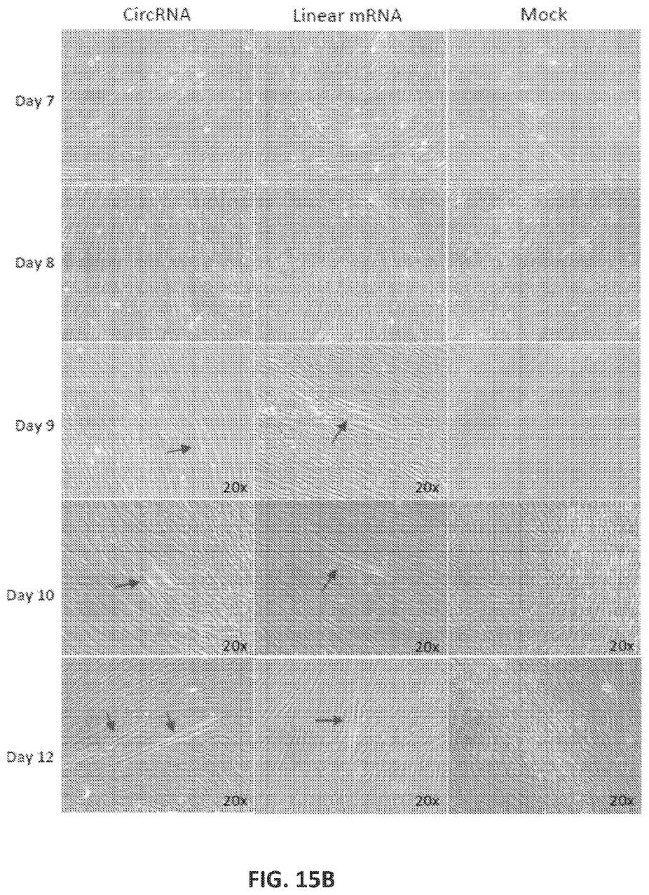

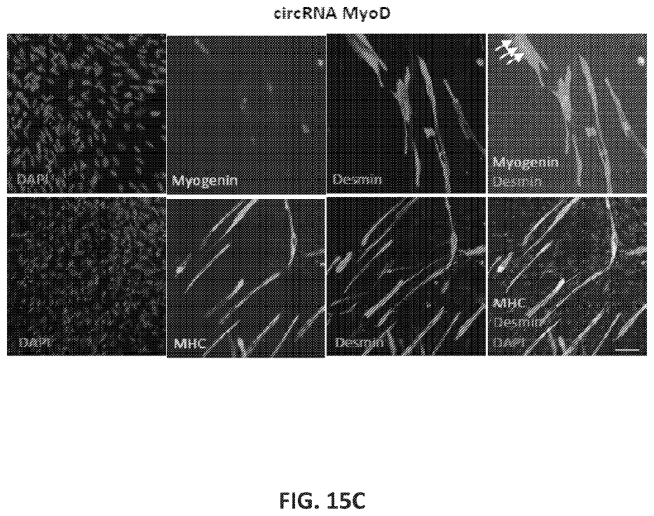

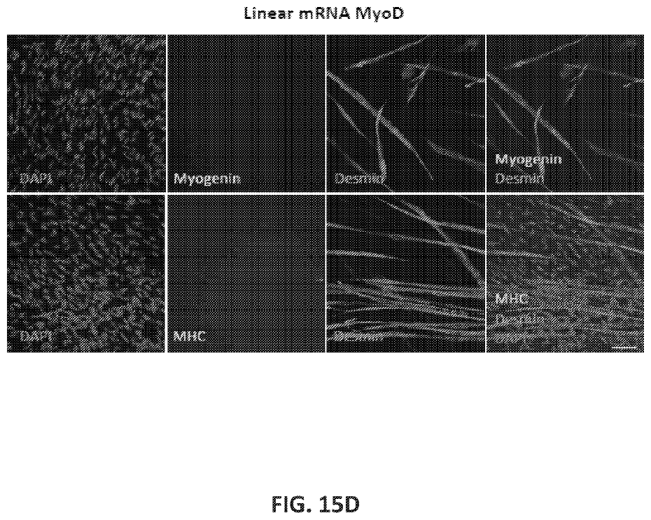

Results are shown in FIG. 15A-FIG. 15C. FIG. 15A shows MyoD expression in transduced cells. Both circRNA and linear mRNA transfected cultures stained positive for MyoD protein, while mock-transfected cultures did not, validating protein expression by both types of RNA. At 24 hrs post transfection, the amount of protein expressed by circRNA was lower than that by linear mRNA.

On day 6 following the final transfection, culture media was changed to reduced serum (from 10% to 2% serum) to induce myoblast fusion and formation of multinucleated myotubes. Phase contrast images shown in FIG. 15B show examples of myotubes (arrows) that were observed in both circRNA MyoD-transfected and linear mRNA MyoD-transfected cultures.

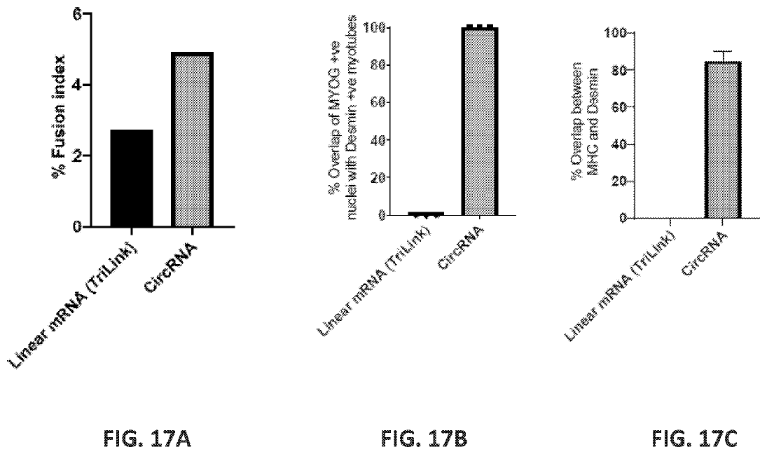

FIG. 15C and FIG. 15D show expression of muscle-specific markers in MyoD-transfected cultures. Myotubes derived from circRNA MyoD-transfected cultures (FIG. 15C) expressed the muscle-specific markers Myogenin, Desmin, and myosin heavy chain (MHC). Arrows in the merged image for Myogenin and Desmin indicate multinucleated fused cells. However, myotubes derived from linear mRNA MyoD-transfected cultures expressed Desmin, but not Myogeninor MHC (FIG. 15D). Data from this experiment is quantified in FIG. 17A-17C.

Desmin, myogenin, and myosin heavy chain (MHC) are generally considered as early, intermediate and late muscle differentiation markers, respectively. The observation that circRNA MyoD-induced myotubes expressed all three markers, while linear mRNA MyoD-induced myotubes expressed only Desmin, but not Myogenin or MHC, indicated that circRNA MyoD resulted in more terminal muscle differentiation than linear mRNA within the same timeframe (12 days).

Taken together, this data is indicative of better overall survival of the cells in culture early during reprogramming (day 6, see FIG. 13), and after complete reprogramming (see, e.g., FIG. 9D, and FIG. 9F (compare wells at 25K for Stemgent, linear and circRNA); see also FIG. 14A (compare wells at 25K for Stemgent, linear and circRNA when 4 transfections (+ or −EKB) were performed) and FIG. 14B).

### Example 10: Use of a Circular RNA in a Combination Method for Reprogramming and Editing the Genome of a Cell

A composition is prepared, the composition comprising (i) recombinant circular RNAs each comprising a sequence encoding at least one reprogramming factor, (ii) nucleic acid encoding a Cas9 nuclease, and (iii) a nucleic acid encoding a gRNA targeting a sequence of interest. The composition is contacted with a cell. The Cas9 edits the DNA of the cell, at the sequence of interest. The reprogramming factor reprograms the cell to a pluripotent state. Accordingly, the genotype and the phenotype of the cell are altered.

### Example 11: Use of a Circular RNA in a Combination Method for Transdifferentiating and Editing the Genome of a Cell

A composition is prepared, the composition comprising (i) recombinant circular RNAs each comprising a sequence encoding at least one transdifferentiation factor, (ii) nucleic acid encoding a Cas9 nuclease, and (iii) a nucleic acid encoding a gRNA targeting a sequence of interest. The composition is contacted with a differentiated cell. The Cas9 edits the DNA of the cell, at the sequence of interest. The transdifferentiation factor reprograms the differentiated cell to be a different differentiated cell type. Accordingly, the genotype and the phenotype of the cell are altered.

The foregoing is illustrative of the present invention, and is not to be construed as limiting thereof. The invention is defined by the following claims, with equivalents of the claims to be included therein.

### REFERENCES

1. Cell Stem Cell (2010) 7: 618
2. SCIENTIFIC REPORTS (2012) 2: 657
3. Nature Review Genetics (2019) 20:675
4. NATURE COMMUNICATIONS (2017) 8: 1149

What is claimed is:

1. A recombinant circular RNA comprising a protein-coding sequence, wherein the protein-coding sequence encodes at least one reprogramming factor, wherein the at least one reprogramming factor is Oct3/4, Klf4, Sox2, Nanog, Lin28, c-Myc, or L-Myc, or a fragment or variant thereof.

2. The recombinant circular RNA of claim 1, wherein the at least one reprogramming factor is a human or a humanized reprogramming factor.

3. The recombinant circular RNA of claim 1 or 2, wherein the at least one reprogramming factor is Oct3/4, and wherein the Oct3/4 has the sequence of SEQ ID NO: 1, or a sequence at least 90% or at least 95% identical thereto.

4. The recombinant circular RNA of claim 3, wherein the recombinant circular RNA comprises a nucleic acid sequence of SEQ ID NO: 33, or a sequence at least 90% or at least 95% identical thereto.

5. The recombinant circular RNA of claim 1 or 2, wherein the at least one reprogramming factor is Klf4, and wherein the Klf4 has the sequence of SEQ ID NO: 2 or 3, or a sequence at least 90% or at least 95% identical thereto.

6. The recombinant circular RNA of claim 4, wherein the recombinant circular RNA comprises a nucleic acid sequence of SEQ ID NO: 37, or a sequence at least 90% or at least 95% identical thereto.

7. The recombinant circular RNA of claim 1 or 2, wherein the at least one reprogramming factor is Sox2, and wherein Sox2 has the sequence of SEQ ID NO: 4, or a sequence at least 90% or at least 95% identical thereto.

8. The recombinant circular RNA of claim 7, wherein the recombinant circular RNA comprises a nucleic acid sequence of SEQ ID NO: 34, or a sequence at least 90% or at least 95% identical thereto.

9. The recombinant circular RNA of claim 1 or 2, wherein the at least one reprogramming factor is Nanog, and wherein the Nanog has the sequence of SEQ ID NO: 5 or 6, or a sequence at least 90% or at least 95% identical thereto.

10. The recombinant circular RNA of claim 9, wherein the recombinant circular RNA comprises a nucleic acid sequence of SEQ ID NO: 36, or a sequence at least 90% or at least 95% identical thereto.

11. The recombinant circular RNA of claim 1 or 2, wherein the at least one reprogramming factor is Lin28, and wherein the Lin28 has the sequence of SEQ ID NO: 7, or a sequence at least 90% or at least 95% identical thereto.

12. The recombinant circular RNA of claim 11, wherein the recombinant circular RNA comprises a nucleic acid sequence of SEQ ID NO: 35, or a sequence at least 90% or at least 95% identical thereto.

13. The recombinant circular RNA of claim 1 or 2, wherein the at least one reprogramming factor is c-Myc, and wherein the c-Myc has the sequence of SEQ ID NO: 8 or 9, or a sequence at least 90% or at least 95% identical thereto.

14. The recombinant circular RNA of claim 13, wherein the recombinant circular RNA comprises a nucleic acid sequence of SEQ ID NO: 38, or a sequence at least 90% or at least 95% identical thereto.

15. The recombinant circular RNA of claim 1 or 2, wherein the at least one reprogramming factor is L-Myc, and wherein the L-Myc has the sequence of any one of SEQ ID NO: 10-12, or a sequence at least 90% or at least 95% identical thereto.

16. The recombinant circular RNA of any one of claims 1-15, wherein the circular RNA is substantially non-immunogenic.

17. The recombinant circular RNA of claim 16, wherein the circular RNA comprises one or more M-6-methyladenosine (m⁶A) residues.

18. The recombinant circular RNA of any one of claim 1-17, wherein the circular RNA comprises from about 200 nucleotides to about 5,000 nucleotides.

19. The recombinant circular RNA of any one of claims 1-18, wherein the circular RNA comprises an internal ribosome entry site (IRES) operably linked to the protein-coding sequence.

20. A complex comprising a recombinant circular RNA of any one of claims 1-19, and a lipid nanoparticle (LNP).

21. The complex of claim 20, wherein the LNP comprises a cationic lipid.

22. The complex of claim 20 or 21, wherein the recombinant circular RNA and the LNP are conjugated.

23. The complex of claim 22, wherein the recombinant circular RNA and the LNP are covalently conjugated.

24. The complex of claim 22, wherein the recombinant circular RNA and the LNP are non-covalently conjugated.

25. A vector comprising a nucleic acid encoding the recombinant circular RNA of any one of claims 1-19.

26. The vector of claim 25, wherein the vector is a non-viral vector.

27. The vector of claim 26, wherein the non-viral vector is a plasmid.

28. The vector of claim 25, wherein the vector is a viral vector.

29. The vector of claim 28, wherein the viral vector is a retroviral vector, a herpesvirus vector, an adenovirus vector, an adeno-associated virus (AAV) vector, a baculoviral vector, an alphavirus vector, a picornavirus vector, a vaccinia virus vector, or a lentiviral vector.

30. A composition comprising the recombinant circular RNA of any one of claims 1-19, the complex of any one of claims 20-24, or the vector of any one of claims 25-29.

31. The composition of claim 30, wherein the composition comprises a carrier and/or a vehicle.

32. A composition comprising two or more of the recombinant circular RNAs of any one of claims 1-19, wherein the composition comprises a combination of recombinant circular RNAs encoding the reprogramming factors selected from those in Table 2.

33. A composition comprising two or more recombinant circular RNAs, wherein the composition comprises a combination of recombinant circular RNAs encoding the reprogramming factors selected from:
(i) Oct3/4, Klf4, Sox2, and c-Myc;
(ii) Oct3/4, Klf4, Sox2, and L-Myc;
(iii) Oct3/4, Klf4, and Sox2;
(iv) Oct3/4, Klf4, Sox2, Nanog, Lin28, and c-Myc; or
(iv) Oct3/4, Klf4, Sox2, Nanog, Lin28, and L-Myc.

34. A kit comprising the recombinant circular RNA of any one of claims 1-19, the complex of any one of claims 20-24, the vector of any one of claims 25-29, or the composition of any one of claims 30-33.

35. A cell comprising the recombinant circular RNA of any one of claims 1-19, the complex of any one of claims 20-24, the vector of any one of claims 25-29, or the composition of any one of claims 30-33.

36. The cell of claim 35, wherein the cell is a eukaryotic cell.

37. The cell of claim 36, wherein the cell is a mammalian cell.

38. The cell of claim 37, wherein the cell is a human cell.

39. The cell of any one of claims 35-38, wherein the cell is a CD34+ cell.

40. A method of expressing a protein in a cell, the method comprising contacting the cell with the circular RNA of any one of claims 1-19, the complex of any one of claims 20-24, the vector of any one of claims 25-29, or the composition of any one of claims 30-33, and maintaining the cell under conditions under which the protein is expressed.

41. The method of claim 40, wherein the method comprises contacting the cell with an additional circular RNA, wherein the additional circular RNA is circBIRC6, circCORO1C, or circMAN1A2.

42. The method of claim 41, wherein the additional circular RNA is circBIRC6, and wherein the circBIRC6 has a sequence of SEQ ID NO: 13, or a sequence at least 90% or at least 95% identical thereto.

43. The method of claim 41, wherein the additional circular RNA is circCORO1C, and wherein the circCORO1C has a sequence of SEQ ID NO: 14, or a sequence at least 90% or at least 95% identical thereto.

44. The method of claim 41, wherein the additional circular RNA is circMAN1A2, and wherein the circMAN1A2 has a sequence of SEQ ID NO: 15, or a sequence at least 90% or at least 95% identical thereto.

45. The method of any one of claims 40-44, wherein the method comprises contacting the cell with a circular RNA encoding B18R.

46. The method of claim 45, wherein the B18R has a sequence of SEQ ID NO: 16, or a sequence at least 90% or at least 95% identical thereto.

47. A method of producing an induced pluripotent stem cell (iPSC), the method comprising contacting a somatic cell with at least one of the recombinant circular RNAs of any one of claims 1-19, the complex of any one of claims 20-24, the vector of any one of claims 25-29, and/or the composition of any one of claims 30-33, and maintaining the cell under conditions under which a reprogrammed iPSC is obtained.

48. The method of claim 47, wherein the method comprises contacting the cell with at least 2, at least 3, at least 4, at least 5, at least 6, at least 7, at least 8, or at least 9 circular RNAs.

49. The method of claim 47 or 48, wherein the method comprises contacting the cell with a first circular RNAs encoding Oct4, a second circular RNA encoding Sox2, a third circular RNA encoding Klf4, a fourth circular RNA encoding C-Myc or L-Myc, and a fifth circular RNA encoding Lin28.

50. The method of claim 47 or 48, wherein the method comprises contacting the cell with a first circular RNAs encoding Oct4, a second circular RNA encoding Sox2, a third circular RNA encoding Klf4, a fourth circular RNA encoding C-Myc or L-Myc, a fifth circular RNA encoding Lin28, and a sixth circular RNA encoding Nanog.

51. The method of claim 47, comprising contacting the somatic cell with at least one non-circular RNA nucleic acids encoding one or more reprogramming factors.

52. The method of claim 51, wherein the one non-circular RNA nucleic acids are selected from an mRNA or a plasmid.

53. The method of any one of claims 47-52, wherein the method comprises contacting the cell with at least one additional circular RNA, wherein the at least one additional circular RNA is circBIRC6, circCORO1C, or circMAN1A2.

54. The method of claim 53, wherein the at least one additional circular RNA is BIRC6, and wherein BIRC6 has a sequence of SEQ ID NO: 13, or a sequence at least 90% or at least 95% identical thereto.

55. The method of claim 53, wherein the at least one additional circular RNA is CORO1C, and wherein CORO1C has a sequence of SEQ ID NO: 14, or a sequence at least 90% or at least 95% identical thereto.

56. The method of claim 53, wherein the at least one additional circular RNA is MAN1A2, and wherein MAN1A2 has a sequence of SEQ ID NO: 15, or a sequence at least 90% or at least 95% identical thereto.

57. The method of any one of claims 47-56, wherein the method comprises contacting the cell with a circular RNA encoding B18R.

58. The method of claim 57, wherein the B18R has a sequence of SEQ ID NO: 16, or a sequence at least 90% or at least 95% identical thereto.

59. The method of any one of claims 47-58, wherein the cell is a fibroblast, a peripheral blood-derived cell, an endothelial progenitor cell, a cord-blood derived cell, a keratinocyte, a melanocyte, an adipose-tissue derived cell, or a urine-derived cell.

60. The method of any one of claims 47-58, wherein the cell is a CD34+ cell.

61. The method of any one of claims 47-60, wherein the cell is an adherent cell.

62. The method of any one of claims 47-60, wherein the cell is in suspension.

63. A method of producing an induced pluripotent stem cell (iPSC), the method comprising contacting a CD34+ in suspension cell with at least one of the recombinant circular RNAs of any one of claims 1-19, the complex of any one of claims 20-24, the vector of any one of claims 25-29, and/or the composition of any one of claims 30-33, and maintaining the cell under conditions under which a reprogrammed iPSC is obtained.

64. The method of any one of claims 47-63, wherein the method results in one or more of:
(i) an increase in the number of reprogrammed iPSC present at the end of culture compared to a method of producing an iPSC with one or more linear RNAs;
(ii) an increase in the rate of reprogrammed iPSC maturation compared to a method of producing an iPSC with one or more linear RNAs; and/or
(iii) a decrease in cell toxicity at one or more timepoints during reprogramming compared to a method of producing an iPSC with one or more linear RNAs.

65. The method of any one of claims 47-63, wherein the method results in each of:
(i) an increase in the number of reprogrammed iPSC present at the end of culture compared to a method of producing an iPSC with one or more linear RNAs;
(ii) an increase in the rate of reprogrammed iPSC maturation compared to a method of producing an iPSC with one or more linear RNAs; and
(iii) a decrease in cell toxicity at one or more timepoints during reprogramming compared to a method of producing an iPSC with one or more linear RNAs.

66. The method of any one of claims 47-65, comprising contacting the cell one or more times with at least one of the recombinant circular RNAs of any one of claims 1-19, the complex of any one of claims 20-24, the vector of any one of claims 25-29, and/or the composition of any one of claims 30-33.

67. The method of claim 66, comprising contacting the cell two, three, four, or more times.

68. The method of claim 66, comprising contacting the cell less than four times.

69. The method of claim 66, comprising contacting the cell between 2 and 4 times.

70. An iPSC produced using the method of any one of claims 47-69.

71. A differentiated cell derived from the iPSC of claim 70.

72. The differentiated cell of claim 71, wherein the differentiated cell is a muscle cell, a neuron, a cardiomyocyte, a hepatocyte, an islet cell, a keratinocyte, a T-cell, or a NK-cell.

73. A method of directly converting a cell from a first cell type to a second cell type, the method comprising contacting the cell with the recombinant circular RNA of any one of claims 1-19, the complex of any one of claims 20-24, the vector of any one of claims 25-29, and/or the composition of any one of claims 30-33, and maintaining the cell under conditions under which the cell is converted to the second cell type.

74. The method of claim 73, wherein the first cell type is a somatic cell and the second cell type is a somatic cell.

75. The method of claim 73, wherein the second cell type is a muscle cell, a neuron, a cardiomyocyte, a hepatocyte, an islet, a keratinocyte, a T-cell, or a NK-cell.

76. The method of claim 74 or 75, wherein the first cell type is a fibroblast.

77. The method of claim 76, wherein the cell is contacted with a plurality of recombinant circular RNAs, wherein the plurality of circular RNAs comprise circular RNAs encoding any one of the combinations of transdifferentiation factors listed in Column B of Table 6.

78. The method of any one of claims 73-77, wherein the cell does not enter an intermediate pluripotent state.

79. The method of any one of claims 73-78, wherein the cell is converted directly from the first cell type to the second cell type, without becoming a progenitor cell.

80. The method of any one of claims 78 to 79, wherein the first type of cell is a fibroblast, the second type of cell is a muscle cell, and the recombinant circular RNA encodes myoD.

81. A cell produced by the method of any one of claims 73 to 80.

82. A method for reprogramming and editing the genome of a cell, the method comprising:
contacting the cell with:
(i) a recombinant circular RNA comprising a protein-coding sequence, wherein the protein-coding sequence encodes at least one reprogramming factor, and
(ii) an enzyme capable of editing the DNA or RNA of the cell, or a nucleic acid encoding the same.

83. The method of claim 82, wherein the recombinant circular RNA is the recombinant circular RNA of any one of claims 1-19.

84. The method of claim 82 or 83, wherein the enzyme is a TALEN, a NgAgo, a SGN, or a RGN, or a modified or truncated variant thereof.

85. The method of any one of claims 82-84, wherein the enzyme is a Cas9 nuclease, a Cas12(a) nuclease (Cpf1), a Cas12b nuclease, a Cas12c nuclease, a TrpB-like nuclease, a Cas13a nuclease (C2c2), a Cas13b nuclease, a Cas 14 nuclease or a modified or truncated variant thereof.

86. The method of claim 85, wherein the enzyme is a Cas9 nuclease, and the Cas9 nuclease is isolated or derived from S. pyogenes or S. aureus.

87. The method of claim 82 or 83, wherein the enzyme is an ADAR.

88. The method of any one of claims 82-84, wherein the enzyme is a RNA-guided nuclease.

89. The method of claim 88, wherein the RNA-guided nuclease is selected from any one of APG05083.1, APG07433.1, APG07513.1, APG08290.1, APG05459.1, APG04583.1, and APG1688.1, APG05733.1, APG06207.1, APG01647.1, APG08032.1, APG05712.1, APG01658.1, APG06498.1, APG09106.1, APG09882.1, APG02675.1, APG01405.1, APG06250.1, APG06877.1, APG09053.1, APG04293.1, APG01308.1, APG06646.1, APG09748, APG07433.1, APG00969, APG03128, APG09748, APG00771, APG02789, APG09106, APG02312, APG07386, APG09980, APG05840, APG05241, APG07280, APG09866, and APG00868.

90. The method of any one of claims 82-89, wherein the method further comprises contacting the cell with a guide RNA, or a nucleic acid encoding the same.

91. The method of any one of claims 82-90, wherein the cell is contacted with the recombinant circular RNA before it is contacted with the enzyme or the nucleic acid encoding the same.

92. The method of any one of claims 82-90, wherein the cell is contacted with the recombinant circular RNA after it is contacted with the enzyme or the nucleic acid encoding the same.

93. The method of any one of claims 82-90, wherein the cell is contacted with the recombinant circular RNA at approximately the same time that it is contacted with the enzyme or the nucleic acid encoding the same.

94. A cell generated by the method of any one of claims 82-93.

95. A method for transdifferentiating and editing the genome of a cell, the method comprising:
contacting the cell with:
(i) a recombinant circular RNA comprising a protein-coding sequence, wherein the protein-coding sequence encodes at least one transdifferentiation factor, and
(ii) an enzyme capable of editing the DNA or RNA of the cell, or a nucleic acid encoding the same.

96. The method of claim 95, wherein the at least one transdifferentiation factor is any one of MyoD, C/EBPα, C/EBPβ, Pdx1, Ngn3, Mafa, Pdx1, Hnf4α, Foxa1, Foxa2, Foxa3, Ascl1 (also known as Mash1), Brn2, Myt1l, miR-124, Brn2, Myt1l, Ascl1, Nurr1, Lmx1a, Ascl1, Brn2, Myt1l, Lmx1a, FoxA2, Oct4, Sox2, Klf4 and c-Myc, Tbx5, Mef2c, Gata-4, or Mesp1.

97. The method of claim 95, wherein the at least one transdifferentiation factor is any one of the transdifferentiation factors listed in Table 6.

98. The method of claim 95, comprising two or more transdifferentiation factors selected from those listed in Table 6.

99. The method of claim any one of claims 95-98, wherein the enzyme is a TALEN, a NgAgo, a SGN, or a RGN, or a modified or truncated variant thereof.

100. The method of any one of claims 95-98, wherein the enzyme is a Cas9 nuclease, a Cas12(a) nuclease (Cpf1), a Cas12b nuclease, a Cas12c nuclease, a TrpB-like nuclease, a Cas13a nuclease (C2c2), a Cas13b nuclease, a Cas 14 nuclease or a modified or truncated variant thereof.

101. The method of claim 100, wherein the nuclease is a Cas9 nuclease, and the Cas9 nuclease is isolated or derived from S. pyogenes or S. aureus.

102. The method of any one of claims 95-98, wherein the enzyme is an ADAR.

103. The method of any one of claims 95-98, wherein the enzyme is a RNA-guided nuclease.

104. The method of claim 103, wherein the RNA-guided nuclease is selected from any one of APG05083.1, APG07433.1, APG07513.1, APG08290.1, APG05459.1, APG04583.1, and APG1688.1, APG05733.1, APG06207.1, APG01647.1, APG08032.1, APG05712.1, APG01658.1, APG06498.1, APG09106.1, APG09882.1, APG02675.1, APG01405.1, APG06250.1, APG06877.1, APG09053.1, APG04293.1, APG01308.1, APG06646.1, APG09748, APG07433.1, APG00969, APG03128, APG09748, APG00771, APG02789, APG09106, APG02312, APG07386, APG09980, APG05840, APG05241, APG07280, APG09866, and APG00868.

105. The method of any one of claims 95-104, wherein the method further comprises contacting the cell with a guide RNA, or a nucleic acid encoding the same.

106. The method of any one of claims 95-105, wherein the cell is contacted with the recombinant circular RNA before it is contacted with the enzyme or the nucleic acid encoding the same.

107. The method of any one of claims 95-105, wherein the cell is contacted with the recombinant circular RNA after it is contacted with the enzyme or the nucleic acid encoding the same.

108. The method of any one of claims 95-105, wherein the cell is contacted with the recombinant circular RNA at approximately the same time that it is contacted with the enzyme or the nucleic acid encoding the same.

109. A cell generated by the method of any one of claims 95-108.

110. A method for reprogramming a cell, the method comprising contacting a cell with one or more of:
(i) a circular RNA encoding a reprogramming factor;
(ii) a circular RNA that does not encode any protein or miRNA;
(iii) a circular or linear RNA encoding a miRNA; and/or
(iv) a circular or linear RNA encoding a viral protein.

111. A method for reprogramming a cell, the method comprising contacting a cell with each of:
(i) a circular RNA encoding a reprogramming factor;
(ii) a circular RNA that does not encode any protein or miRNA;
(iii) a circular or linear RNA encoding a miRNA; and
(iv) a circular or linear RNA encoding a viral protein.

112. A method for reprogramming a cell, the method comprising contacting a cell with each of:
(i) a circular RNA encoding a reprogramming factor;
(ii) a circular or linear RNA encoding a miRNA; and
(iii) a circular or linear RNA encoding a viral protein.

113. A method for reprogramming a cell, the method comprising contacting a cell with each of:
(i) a circular RNA encoding a reprogramming factor; and
(ii) a circular or linear RNA encoding a miRNA.

114. The method of any one of claims 110-113, wherein any one of the circular RNA or linear RNAs are conjugated to a lipid nanoparticle.

115. The method of any one of claims 110-113, wherein the reprogramming factor is any one of the reprogramming factors listed in Table 1, Table 2, or Table 3.

116. The method of any one of claims 110-115, wherein the circular RNA is the recombinant circular RNA of any one of claims 1-19.

117. The method of claim 111, wherein the circular RNA that does not encode any protein or miRNA is circBIRC6, circCORO1c, or circMAN1A2.

118. The method of any one of claims 111-113, wherein the miRNA is miR302d, miR302a, miR302c, miR302b, or miR367.

119. The method of any one of claims 111-113, the miRNA is miR146a, miR485, miR182, nc886, miR-155, miR526a, or miR132.

120. The method of any one of claims 111-112, wherein the viral protein is B18R, E3, or K3.

121. The method of any one of claims 111-112, wherein the viral protein is any one of the viral proteins listed in Table 4.

122. The method of any one of claims 111-112, wherein the viral protein is B18R, E3, and K3.

123. A cell generated by the method of any one of claims 111-122.

124. A composition comprising an isolated somatic cell that comprising one or more exogenous circular RNAs encoding a reprogramming factor.

125. The composition of claim 124, wherein the somatic cell comprises one or more exogenous circular RNAs encoding a reprogramming factor selected from the reprogramming factors listed in Table 1, Table 2, or Table 3.

126. The composition of claim 124, wherein the somatic cell comprises one or more exogenous circular RNAs, wherein the one or more exogenous circular RNAs each encode a reprogramming factor selected from Oct3/4, Klf4, Sox2, Nanog, Lin28, and c-Myc.

127. The composition of claim 124, wherein the somatic cell comprises six exogenous circular RNAs, wherein each circular RNA encodes one of Oct3/4, Klf4, Sox2, Nanog, Lin28, and c-Myc.

128. The composition of claim 124, wherein the somatic cell comprises one or more exogenous circular RNAs, wherein the one or more endogenous circular RNAs each encode a reprogramming factor selected from Oct3/4, Klf4, Sox2, Nanog, Lin28, and L-Myc.

129. The composition of claim 124, wherein the somatic cell comprises six exogenous circular RNAs, wherein each circular RNA encodes one of Oct3/4, Klf4, Sox2, Nanog, Lin28, and L-Myc.

130. The composition of claim 124, wherein the somatic cell comprises four exogenous circular RNAs, wherein each circular RNA encodes one of Oct3/4, Klf4, Sox2, and c-Myc.

131. The composition of claim 124, wherein the somatic cell comprises four exogenous circular RNAs, wherein each circular RNA encodes one of Oct3/4, Klf4, Sox2, and L-Myc.

132. The composition of claim 124, wherein the somatic cell comprises four exogenous circular RNAs, wherein each circular RNA encodes one of Oct3/4, Klf4, and Sox2.

133. The composition of any one of claims 124-132, wherein the cell comprises at least one, at least two, or all three exogenous viral proteins selected from B18R, E3, and K3.

134. The composition of any one of claims 124-133, wherein the cell comprises an exogenous miRNA.

135. The composition of any one of claim 124-133, wherein the cell comprises a circular RNA encoding an exogenous miRNA

136. The composition of claim 134 or 135, wherein the miRNA is selected from miR302a, miR302b, miR302c, miR302d and miR367.

137. A composition comprising a transdifferentiated cell, wherein the transdifferentiated cell comprises one or more exogenous circular RNAs encoding a transdifferentiation factor.

138. The composition of claim 137, wherein the transdifferentiation factor is any one of the transdifferentiation factors or combinations of transdifferentiation factors listed in Table 6.

139. The composition of claim 137 or 138, wherein the transdifferentiated cell is any one of the second cell types listed in Table 6.

140. The composition of claim 137 or 138, wherein the transdifferentiated cell is derived from a first cell type that is any one of the first cell types listed in Table 6.

141. A method of reprogramming a cell which produces reduced cell death as compared to a method using linear RNA, the method comprising contacting a cell with the circular RNA of any one of claims 1-19, the complex of any one of claims 20-24, the vector of any one of claims 25-29, or the composition of any one of claims 30-33, and maintaining the cell under conditions under which the protein is expressed.

142. The method of claim 141, wherein the method comprises contacting the cell with a plurality of circular RNAs, wherein each circular RNA encodes one of the following reprogramming factors:
(i) Oct3/4, Klf4, Sox2, Nanog, Lin28, and c-Myc;
(ii) Oct3/4, Klf4, Sox2, Nanog, and Lin28;
(iii) Oct3/4, Klf4, Sox2, Nanog, Lin28, and L-Myc;
(iv) Oct3/4, Klf4, Sox2, Nanog, and Lin28;
(v) Oct3/4, Klf4, Sox2, and c-Myc;
(vi) Oct3/4, Klf4, Sox2, and L-Myc; or
(vii) Oct3/4, Klf4, and Sox2.

143. A method of reducing time from reprogramming to picking, the method comprising contacting a cell with the circular RNA of any one of claims 1-19, the complex of any one of claims 20-24, the vector of any one of claims 25-29, or the composition of any one of claims 30-33, and maintaining the cell under conditions under which the protein is expressed, wherein the time is reduced relative to a reprogramming method using linear RNA.

144. The method of claim 143, wherein the method comprises contacting the cell with a plurality of circular RNAs, wherein each circular RNA encodes one of the following reprogramming factors:
(i) Oct3/4, Klf4, Sox2, Nanog, Lin28, and c-Myc;
(ii) Oct3/4, Klf4, Sox2, Nanog, and Lin28;
(iii) Oct3/4, Klf4, Sox2, Nanog, Lin28, and L-Myc;
(iv) Oct3/4, Klf4, Sox2, Nanog, and Lin28;
(v) Oct3/4, Klf4, Sox2, and c-Myc;
(vi) Oct3/4, Klf4, Sox2, and L-Myc; or
(vii) Oct3/4, Klf4, and Sox2.

145. A method of reducing the number of transfections induce to effect reprogramming of a cell, the method comprising contacting a cell with the circular RNA of any one of claims 1-19, the complex of any one of claims 20-24, the vector of any one of claims 25-29, or the composition of any one of claims 30-33, and maintaining the cell under conditions under which the protein is expressed, relative to a method using linear RNA.

146. The method of claim 145, wherein the method comprises contacting the cell with a plurality of circular RNAs, wherein each circular RNA encodes one of the following reprogramming factors:
(i) Oct3/4, Klf4, Sox2, Nanog, Lin28, and c-Myc;
(ii) Oct3/4, Klf4, Sox2, Nanog, and Lin28;
(iii) Oct3/4, Klf4, Sox2, Nanog, Lin28, and L-Myc;
(iv) Oct3/4, Klf4, Sox2, Nanog, and Lin28;
(v) Oct3/4, Klf4, Sox2, and c-Myc;
(vi) Oct3/4, Klf4, Sox2, and L-Myc; or
(vii) Oct3/4, Klf4, and Sox2.

147. A method of increasing duration of protein expression in a cell, the method comprising contacting a cell with the circular RNA of any one of claims 1-19, the complex of any one of claims 20-24, the vector of any one of claims 25-29, or the composition of any one of claims 30-33, and maintaining the cell under conditions under which the protein is expressed, and wherein the duration of protein expression is increased relative to transfection of the cell with a linear RNA encoding the same protein.

148. The method of claim 147, wherein the method comprises contacting the cell with a plurality of circular RNAs, wherein each circular RNA encodes one of the following reprogramming factors:
(i) Oct3/4, Klf4, Sox2, Nanog, Lin28, and c-Myc;
(ii) Oct3/4, Klf4, Sox2, Nanog, and Lin28;
(iii) Oct3/4, Klf4, Sox2, Nanog, Lin28, and L-Myc;
(iv) Oct3/4, Klf4, Sox2, Nanog, and Lin28;
(v) Oct3/4, Klf4, Sox2, and c-Myc;
(vi) Oct3/4, Klf4, Sox2, and L-Myc; or
(vii) Oct3/4, Klf4, and Sox2.

149. A method of improving cellular reprogramming efficiency, the method comprising contacting a cell with the circular RNA of any one of claims 1-19, the complex of any one of claims 20-24, the vector of any one of claims 25-29, or the composition of any one of claims 30-33, and maintaining the cell under conditions under which the protein is expressed, and wherein the efficacy of cellular reprogramming is increased relative to a cellular reprogramming method in which linear RNA is used.

150. The method of claim 149, wherein the method comprises contacting the cell with a plurality of circular RNAs, wherein each circular RNA encodes one of the following reprogramming factors:
(i) Oct3/4, Klf4, Sox2, Nanog, Lin28, and c-Myc;
(ii) Oct3/4, Klf4, Sox2, Nanog, and Lin28;
(iii) Oct3/4, Klf4, Sox2, Nanog, Lin28, and L-Myc;
(iv) Oct3/4, Klf4, Sox2, Nanog, and Lin28;
(v) Oct3/4, Klf4, Sox2, and c-Myc;
(vi) Oct3/4, Klf4, Sox2, and L-Myc; or
(vii) Oct3/4, Klf4, and Sox2.

151. A method of increasing the number of reprogrammed cell colonies formed after reprogramming, the method comprising contacting a cell with the circular RNA of any one of claims 1-19, the complex of any one of claims 20-24, the vector of any one of claims 25-29, or the composition of any one of claims 30-33, and maintaining the cell under conditions under which the protein is expressed, wherein the number of reprogrammed cell colonies formed after reprogramming is increased relative to a cellular reprogramming method in which linear RNA is used.

152. The method of claim 151, wherein the method comprises contacting the cell with a plurality of circular RNAs, wherein each circular RNA encodes one of the following reprogramming factors:
(i) Oct3/4, Klf4, Sox2, Nanog, Lin28, and c-Myc;
(ii) Oct3/4, Klf4, Sox2, Nanog, and Lin28;
(iii) Oct3/4, Klf4, Sox2, Nanog, Lin28, and L-Myc;
(iv) Oct3/4, Klf4, Sox2, Nanog, and Lin28;
(v) Oct3/4, Klf4, Sox2, and c-Myc;
(vi) Oct3/4, Klf4, Sox2, and L-Myc; or
(vii) Oct3/4, Klf4, and Sox2.

153. A method of reprogramming cells in suspension, the method comprising contacting a cell in suspension with the circular RNA of any one of claims 1-19, the complex of any one of claims 20-24, the vector of any one of claims 25-29, or the composition of any one of claims 30-33, and maintaining the cell under conditions under which the protein is expressed.

154. The method of claim 153, wherein the method comprises contacting the cell in suspension with a plurality of circular RNAs, wherein each circular RNA encodes one of the following reprogramming factors:
(i) Oct3/4, Klf4, Sox2, Nanog, Lin28, and c-Myc;
(ii) Oct3/4, Klf4, Sox2, Nanog, and Lin28;
(iii) Oct3/4, Klf4, Sox2, Nanog, Lin28, and L-Myc;
(iv) Oct3/4, Klf4, Sox2, Nanog, and Lin28;
(v) Oct3/4, Klf4, Sox2, and c-Myc;
(vi) Oct3/4, Klf4, Sox2, and L-Myc; or
(vii) Oct3/4, Klf4, and Sox2.

155. The method of claim 153 or 154, wherein the cell expresses CD34.

156. A method of improving morphological maturation of reprogrammed colonies, the method comprising contacting a cell in suspension with the circular RNA of any one of claims 1-19, the complex of any one of claims 20-24, the vector of any one of claims 25-29, or the composition of any one of claims 30-33, and maintaining the cell under conditions under which the protein is expressed, wherein the morphological maturation is improved relative to a cellular reprogramming method in which linear RNA is used.

157. The method of claim 156, wherein the method comprises contacting the cell in suspension with a plurality of circular RNAs, wherein each circular RNA encodes one of the following reprogramming factors:
(i) Oct3/4, Klf4, Sox2, Nanog, Lin28, and c-Myc;
(ii) Oct3/4, Klf4, Sox2, Nanog, and Lin28;
(iii) Oct3/4, Klf4, Sox2, Nanog, Lin28, and L-Myc;
(iv) Oct3/4, Klf4, Sox2, Nanog, and Lin28;
(v) Oct3/4, Klf4, Sox2, and c-Myc;
(vi) Oct3/4, Klf4, Sox2, and L-Myc; or
(vii) Oct3/4, Klf4, and Sox2.

158. A suspension culture comprising one or more CD34-expressing cells, wherein the CD34-expressing cells comprise one or more exogenous circRNAs encoding a reprogramming factor.

159. The suspension culture of claim 158, wherein the reprogramming factor is selected from Oct3/4, Klf4, Sox2, Nanog, Lin28, c-Myc, and L-Myc.

160. The suspension culture of claim 158, wherein the CD34-expressing cells each comprise a plurality of circRNAs encoding one of the following combinations of reprogramming factors:
(i) Oct3/4, Klf4, Sox2, Nanog, Lin28, and c-Myc;
(ii) Oct3/4, Klf4, Sox2, Nanog, and Lin28;
(iii) Oct3/4, Klf4, Sox2, Nanog, Lin28, and L-Myc;
(iv) Oct3/4, Klf4, Sox2, Nanog, and Lin28;
(v) Oct3/4, Klf4, Sox2, and c-Myc;
(vi) Oct3/4, Klf4, Sox2, and L-Myc; or
(vii) Oct3/4, Klf4, and Sox2.

161. A kit comprising:
(i) a vessel comprising a circular RNA encoding OCT4 and a buffer;
(ii) a vessel comprising a circular RNA encoding SOX2 and a buffer;
(iii) a vessel comprising a cirRNA encoding KLF4 and a buffer; and
(iv) packaging and instructions therefor.

162. The kit of claim 161, wherein the kit comprises:
a vessel comprising a circular RNA encoding c-MYC or L-MYC and a buffer;
a vessel comprising a cirRNA encoding LIN28 and a buffer;
a vessel comprising a cirRNA encoding NANOG and a buffer;
or a combination thereof.

163. A method for inducing a mesenchymal-to-epithelial transition (MET) of a somatic cell to an iPSC comprising contacting the somatic cell with one or more circular RNA encoding a reprogramming factor.

164. A method for inducing a mesenchymal-to-epithelial transition (MET) of a somatic cell to an iPSC comprising contacting the somatic cell with one or more circular RNA encoding a reprogramming factor.

165. A method for transdifferentiating a cell, the method comprising contacting the cell with a recombinant circular RNA comprising a protein-coding sequence, wherein the protein-coding sequence encodes at least one transdifferentiation factor.

166. The method of claim 165, wherein the at least one transdifferentiation factor is any one of MyoD, C/EBPα, C/EBPβ, Pdx1, Ngn3, Mafa, Pdx1, Hnf4α, Foxa1, Foxa2, Foxa3, Ascl1 (also known as Mash1), Brn2, Myt1l, miR-124, Brn2, Myt1l, Ascl1, Nurr1, Lmx1a, Ascl1, Brn2, Myt1l, Lmx1a, FoxA2, Oct4, Sox2, Klf4 and c-Myc, Tbx5, Mef2c, Gata-4, or Mesp1.

167. The method of claim 165, wherein the at least one transdifferentiation factor is any one of the transdifferentiation factors listed in Table 6.

168. The method of claim 167, wherein the cell is contacted with circular RNAs encoding the combination of transdifferentiation factors listed in any one of the combinations shown in Column B of Table 6.

169. A method for differentiating an iPSC, the method comprising contacting the iPSC with a circular RNA encoding one or more of the following differentiation factors: RORA, HLF, MYB, KLF4, ERG, SOX4, LUC, HOXA9, HOXA10, or HOXA5.

170. The method of claim 169, wherein the iPSC is differentiated to a T-cell.

171. A cell generated by the method of any one of claims 165-170.
